(19) United States
(12) Patent Application Publication
(10) Pub. No.: US 2025/0345483 A1
(43) Pub. Date: Nov. 13, 2025

(54) WIDE RANGE SYNTHETIC PEPTIDE NUCLEIC ACIDS AND THEIR APPLICATIONS

(71) Applicants: Carilion Clinic, Roanoke, VA (US); Virginia Tech Intellectual Properties, Inc., Blacksburg, VA (US)

(72) Inventors: Jayasimha Rao, Roanoke, VA (US); Nammalwar Sriranganathan, Blacksburg, VA (US); Elizabeth Nowak, Hardy, VA (US); Michael Schulz, Christiansburg, VA (US); Hannah Karp, Roanoke, VA (US); Gillian Su, Blacksburg, VA (US)

(21) Appl. No.: 19/201,032

(22) Filed: May 7, 2025

Related U.S. Application Data
(60) Provisional application No. 63/643,580, filed on May 7, 2024.

(51) Int. Cl. A61L 27/34; A61L 27/54; C08L 79/04; C12N 15/113

(52) U.S. Cl. CPC A61L 27/34; A61L 27/54; C08L 79/04; C12N 15/113; A61L 2300/258; A61L 2300/406; A61L 2420/02; C12N 2310/11; C12N 2310/3513

(57) ABSTRACT

Peptide nucleic acids, compositions containing the peptide nucleic acids, and methods utilizing the peptide nucleic acids are described. The peptide nucleic acids, compositions, and methods are designed to inhibit global regulatory bacterial genes and useful for preventing biofilm formation on biotic and abiotic surfaces. The PNAs are designed to account for nuanced interspecies variations in these regulatory genes, thus allowing for simultaneous inhibition of multiple bacterial species as well as polymicrobial biofilms. The disclosure outlines the composition of wide range PNAs (wrPNAs) and applications for delivery.

### CROSS-REFERENCE TO RELATED APPLICATIONS

The present application relies on the disclosure of and claims priority to and the benefit of the filing date of U.S. Provisional Application No. 63/643,580, filed May 7, 2024, the disclosure of which is hereby incorporated by reference herein in its entirety.

### SEQUENCE LISTING

The instant application contains a Sequence Listing in XML format which has been submitted via the USPTO patent electronic filing system and is hereby incorporated by reference in its entirety. Said XML copy, created on May 5, 2025, is named VTC_PNA_F and is 45,032 bytes in size.

### FIELD

The present disclosure is directed to the field of genetic engineering and molecular biology. More specifically, the present disclosure pertains to the design and use of antisense peptide nucleic acids (PNAs) for medical and industrial applications such as preventing the formation of biofilms.

### BACKGROUND

Biofilms are three-dimensional communities of microorganisms that irreversibly adhere to surfaces. They consist of microbial colonies encased in self-produced extracellular polymeric substances (EPS) (Flemming et al., 2016; Stewart and Costerton, 2001). Biofilms are able to share resources, communicate, and self-regulate density through quorum sensing (Arciola et al., 2018; Eze et al., 2018; Flemming et al., 2016; Srinivasan et al., 2021; Stewart and Costerton, 2001). Bacteria within biofilms often have lower metabolic rates than their planktonic counterparts, but are able to shed and seed to other areas, thus perpetuating infections (Roy et al., 2018; Williamson et al., 2012). These factors make mature biofilms difficult to eradicate with standard antimicrobial therapies, and they are known for “tenacious survival” even after lengthy and aggressive therapy (Donlan and Costerton, 2002; Orazi and O'Toole, 2019; Roy et al., 2018; Stewart and Costerton, 2001).

Biofilms are widely implicated in chronic infections and device-associated infections. They are seen on biotic surfaces, such as in dental infections, chronic sinusitis, chronic otitis media, bacterial vaginosis, and cystic fibrosis, as well as abiotic, device-associated infections, such as central line associated bloodstream infections (CLABSI), catheter-associated urinary tract infections (CAUTIs), ventilator tubing, cardiac device related infections, and prosthetic joint related infections (Arciola et al., 2018; Roy et al., 2018; Williamson et al., 2012).

Biofilms are also important outside of medicine, including in water purification, wastewater treatment, sea vessels, and HVAC systems (Flemming et al., 2016; Srinivasan et al., 2021).

Biofilms impact not only morbidity and mortality, but they also pose a financial burden to healthcare systems. For example, the Centers for Disease Control and Prevention estimates each central line associated bloodstream infection (CLABSI) costs approximately $48,000 per episode and carries a mortality risk of up to 25% (“AHRQ's Healthcare-Associated Infections Program,” n.d.). In the United States, over one quarter of all hospital-acquired infections are device-associated, and therefore directly linked to biofilm (Magill et al., 2014).

Pseudomonas aeruginosa is a leading cause of biofilm, and mechanisms of biofilm formation have been studied extensively (Tuon et al., 2022). Pseudomonas biofilms are implicated in CAUTIs, burn wounds, diabetic foot wounds, ventilator associated pneumonia, pneumonia in patients with cystic fibrosis, and prosthetic joint infections (Tuon et al., 2022).

With the exception of Enterococcus faecalis, a majority of all CAUTIs are caused by biofilm forming Gram-negative bacteria, including Escherichia coli, Klebsiella spp., Pseudomonas aeruginosa, Proteus spp., and Enterobacter spp. (Weiner-Lastinger et al., 2020).

Once biofilms have matured, they are extremely difficult to treat with standard antibiotic therapies (Arciola et al., 2018; Stewart and Costerton, 2001). Furthermore, biofilms can be polymicrobial, leading to difficulty selecting effective therapies (Orazi and O'Toole, 2019).

Several strategies are used to prevent biofilm-related infection including early removal of catheters or devices, the use of antimicrobial coatings, systemic antibiotics, and local delivery of antibiotics through lock therapy or cement (Arciola et al., 2018; Ciarolla et al., 2022; Lebeaux et al., 2014; Sharma et al., 2023).

Treatment of biofilms often relies on removing the implicated device, or, in the case of biotic surfaces, surgical debridement of the infected tissue. These approaches can be expensive and invasive (Lebeaux et al., 2014; Sharma et al., 2023). In the case of polymicrobial or recurrent biofilm related infections, it can be difficult to select appropriate regimens that address all the implicated bacteria, especially with the increased risk for multidrug resistant organisms.

### SUMMARY

In embodiments, the disclosure relates generally to designing and using antisense peptide nucleic acids (PNAs) to complement bacterial genes for preventing formation of biofilms through restricting bacterial function. The bacterial genes include regulatory genes found in multiple biofilm-forming bacteria including global regulators rpoS, rsmA, amrZ, and flagellar motility gene motA. The PNA sequences are adjusted to account for interspecies variation, in order to target polymicrobial Gram-negative biofilms. Other embodiments include design of PNAs to target other bacteria including Gram-positive organisms and atypical bacteria.

In general, in a first aspect, the present disclosure features a PNA or combination of PNAs thereof, each of 10 to 20 nucleobases, such as 11 to 15 nucleobases, or 12 to 14 nucleobases, or 13-16 nucleobases, or 12-19 nucleobases, capable of hybridizing through antisense hybridization to a complementary base sequence within, represented by, or inclusive of one or more genomic regions represented in FIG. 1B or any reverse, reverse complementary, or complementary sequence thereof. The PNAs can include a nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof and is capable of hybridizing to its intended target organism such as a polynucleotide of the target organism, such as the complementary base sequences represented in FIG. 1B or any reverse, reverse complementary, or complementary sequence thereof.

In general, in a second aspect, the present disclosure features a pharmaceutical composition including one or more PNAs (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof) within a pharmaceutical carrier. The pharmaceutical carrier can be a solution, suspension, emulsion, ointment, cream, or gel, or can be or include one or more polymer chosen from one or more of a polyester, a polyurethane, a poly(meth)acrylate, a polysaccharide, a polyamide, a polynorbornene, a polycarbonate, a poly(meth)acrylamide, a polyoxazoline, a poly(ethylene oxide), a polyaziridine, or a polysiloxane. The pharmaceutical carrier can be or include polydopamine and can further include one or more antibiotics.

In general, in a third aspect, the present disclosure features a medical device or surface thereof including one or more PNAs of the disclosure (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof), such as a coating containing one or more of such peptide nucleic acids. The coating can also include one or more antibiotics.

In general, in a fourth aspect, the present disclosure features an industrial device or surface thereof including one or more PNAs of the disclosure (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof), such as a coating containing one or more of such peptide nucleic acids. The coating can also include one or more antibiotics.

In general, in a fifth aspect, the present disclosure features a method. The method includes applying, coating, or embedding one or more peptide nucleic acids designed to target or hybridize to one or more bacterial genes comprising rpoS, rsmA, amrZ, and motA (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof) to one or more surfaces within a healthcare setting, to a medical device or surface thereof, or to an industrial device or system or surface thereof.

In general, in a sixth aspect, the present disclosure features a method. The method includes administering one or more peptide nucleic acids designed to target or hybridize to one or more bacterial genes comprising rpoS, rsmA, amrZ, and motA (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof) to a patient. The administration can be through topical administration, such as to a burn wound, diabetic foot wound, mucosal surface, body cavity, or ear canal, or can be through intrapulmonary administration.

In general, in a seventh aspect, the present disclosure features a method. The method is a method of preventing the formation of a biofilm on an abiotic or biotic surface which includes hybridizing or causing to hybridize one or more peptide nucleic acids (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof) to one or more bacterial genes comprising rpoS, rsmA, amrZ, and motA of one or more bacterial organisms present on the abiotic or biotic surface. Causing to hybridize can include applying, coating, embedding, or administering the one or more peptide nucleic acids to the abiotic or biotic surface.

The methods can target one or more bacterial organisms chosen from P. aeruginosa, P. mirabilis, P. vulgaris, E.coli, E. cloacae, and K. pneumoniae. The methods can include applying, coating, embedding, or administering one or more of any of the peptide nucleic acids described or disclosed herein (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof), as well as one or more antibiotics.

In general, in an eighth aspect, the disclosure features a kit. The kit can include a vessel, and one or more peptide nucleic acids of the disclosure (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof) disposed within the vessel.

The PNAs (e.g., one or more nucleobase sequence chosen from SEQ ID NOS:1-10 or any reverse, reverse complementary, or complementary sequence thereof, or a nucleobase sequence that has at least about 80%, 85%, 90%, 95%, 96%, 97%, 98% and 99% sequence identity to a nucleobase sequence chosen from SEQ ID NOS:1-10 or any reverse, reverse complementary, or complementary sequence thereof) can be used singly or in various combinations with each other, and/or with one or more antibiotics, in various embodiments or implementations.

An embodiment or implementation of aspects of the disclosure includes a combination or cocktail of three PNAs (rpoS 0, rsmA 0, amrZ 0) (termed “cocktail of PNAs”, or “cPNAs”) and methods of treating biofilms formed by P. aeruginosa using a cocktail of three PNAs (rpoS 0, rsmA 0, amrZ 0).

Another embodiment or implementation of aspects of the disclosure includes generating broader spectrum PNAs by targeting global regulatory genes that are relatively conserved across microbial species (termed “wide range PNAs” or “wrPNAs”).

Another embodiment or implementation of aspects of the disclosure includes combining wrPNAs to prevent polymicrobial biofilms.

Another embodiment or implementation of aspects of the disclosure includes combining wrPNAs with antibiotics for enhanced bactericidal effect.

Another embodiment or implementation of aspects of the disclosure includes delivery of cPNAs or wrPNAs in various medical applications such as a coating or solution for catheters; cement or coating for prosthetic joints or orthopedic hardware; gastrointestinal, hepatobiliary, pancreatic, genitourinary drains, stents or mesh; cardiac devices or stents; an aerosol for chronic lung disease; or topical application for wounds. The delivery can be combined with antimicrobial compounds such as polydopamine.

Another embodiment or implementation of aspects of the disclosure includes delivery of cPNAs or wrPNAs to environmental surfaces prone to biofilm formation, including HVAC systems and water purification systems.

It should be understood that the peptide nucleic acids, pharmaceutical compositions, medical devices, industrial devices, methods, and kits are not to be considered limitations on the invention defined by the claims. The featured peptide nucleic acids, pharmaceutical compositions, medical devices, industrial devices, methods, and kits can be implemented in one or more ways using one or more features depicted in the drawings, described in the detailed description, and set forth in the claims.

### BRIEF DESCRIPTION OF THE DRAWINGS

The accompanying drawings illustrate certain aspects and principles of the implementations set forth, and should not be construed as limiting.

FIG. 1A is a table showing nucleobase sequences of PNA implementations of the disclosure and intended target(s). The start codon of each sequence (ATG or GTG) is highlighted in red. FIG. 1B demonstrates examples of PNA gene target locations in different bacterial species. FIG. 1C shows an example of PNA synthesis using a cell penetrating peptide (CPP) and an O-linker.

FIGS. 2A-2G are graphs showing that cPNAs and wrPNAs demonstrate biofilm reduction and variable effect on bacterial viability. Biofilm-forming laboratory strain Pseudomonas aeruginosa (PAO1) treated with a cocktail of PNAs (rsmA 0, rpoS 0, and amrZ 0) showed significant reduction in biofilm and cell viability (FIGS. 2A-2G) and served as a basis of comparison for subsequent experiments with wrPNAs. Clinical isolates were obtained from a Gram-negative repository from Carilion Clinic (Roanoke, VA). Klebsiella pneumoniae (KP) was treated with rsmA 1 (FIGS. 2A and 2B); laboratory strain Enterobacter cloacae (ATCC 13047) was treated with a cocktail of rsmA 1 and amrZ 2 (FIGS. 2C and 2D); Escherichia coli (EC) was treated with a cocktail of rsmA 1 and amrZ 1 (FIGS. 2E and 2F); Proteus mirabilis (PM) was treated with a cocktail of rsmA 1, rpoS 1, and motA 1 (FIG. 2G). Biofilm biomass was assessed after 24 hrs (FIGS. 2A-2F) and 48 hrs (FIG. 2G) incubation period. All results are presented as a mean+/−SD (n=3, except FIGS. 2E and 2F n=6). P values were determined using unpaired t-tests (alpha=0.05).

FIGS. 3A-3D are graphs showing the results of experiments with gentamicin-polydopamine coated polystyrene 96-well plates. Biofilm and bacterial viability of gentamicin-sensitive biofilm forming laboratory strain P. aeruginosa (PAO1) and gentamicin resistant Pa383-ΔrahU::GM were assessed after 48 hr incubation in polystyrene 96-well plates coated with lower polydopamine (PDA) concentrations (0.05 or 0.1 mg/mL) (FIGS. 3A and 3B) and higher PDA concentrations (0.25, 0.5, 0.75, or 1.0 mg/mL) (FIGS. 3C and 3D) with constant gentamicin (2 mg/mL). The results are presented as mean+/−SD (n=3). P values determined using one-way ANOVA with follow up tests comparing the mean of each column to the control column (alpha=0.05).

FIG. 4 is a graph showing reduction in biofilm in laboratory strain P. aeruginosa (PAO1) with coated motA PNA. Biofilm measured after 24 hr incubation in polystyrene 96-well plates coated with motA at increasing concentrations. Results are presented as mean+/−SD (n=3). P-values determined using one-way ANOVA with follow up tests comparing the mean of each column to the control column (alpha=0.05).

FIGS. 5A-5D are graphs showing the results of experiments demonstrating antisense-PNAs reduced biofilm formation and bacterial viability among PAO1. FIGS. 5A and 5B show biofilm biomass and bacterial viability after 24 h cPNAs treatment (n=15). FIGS. 5C and 5D show biofilm biomass and bacterial viability after 24 h treatment with antisense-PNA motA 0 (n=9). Results are presented as mean±s.d. and P values were determined using unpaired t-tests (a=0.05).

FIGS. 6A and 6B are images of agar plates and FIGS. 6C and 6D are graphs of results of experiments demonstrating antisense-PNAs reduced biofilm biomass in a polymicrobial sample of 1:1 PAO1:K. pneumoniae. FIG. 6A shows the mucoid phenotype of K. pneumoniae is highlighted on MacConkey (MAC) agar plates compared to Tryptic Soy Agar (TSA) plates. FIG. 6B shows qualitative viability after 24 h antisense-PNA treatment. The graph of FIG. 6C shows biofilm biomass after 24 h antisense-PNA treatment. The graph of FIG. 6D shows bacterial viability after 24 h antisense-PNA treatment. All conditions were tested in triplicate. Results are presented as mean±s.d. and P values were determined using one-way ANOVA with follow up tests comparing the mean of each column to the control column (α=0.05).

FIG. 7A is an image of a plate and FIG. 7B is a graph showing results of experiments demonstrating dried cPNAs retained their anti-biofilm effect against gentamicin-resistant P. aeruginosa, Pa383-ΔrahU::GM. FIG. 7A shows qualitative viability after 24 h incubation, while the graph of FIG. 7B shows biofilm biomass after 32 h cPNAs treatment in aqueous (aq) solution or dried onto the 96-well plate with methanol (MeOH). All conditions were tested in triplicate. Results are presented as mean±s.d. and P values were determined using two-way ANOVA with multiple comparisons comparing cell means with others in its row and its column (family-wise a threshold and confidence level=0.05).

FIGS. 8A-8F are graphs shows the results of experiments demonstrating PAO1 biofilm biomass and bacterial growth after treatment. PAO1 was treated with an antisense PNA of each of the three gene targets: rsmA (FIGS. 8A and 8B),amrZ (FIGS. 8C and 8D), and rpoS (FIGS. 8E and 8F). Biofilm biomass was quantified using crystal violet and static growth was measured at 600 nm with a spectrophotometer. Isolates were incubated for 24 h in duplicate (FIGS. 8A-8F). Results are presented as mean±s.d.

FIGS. 9A-9G are graphs showing results of screening for biofilm-forming phenotypes, defines as an average biofilm biomass greater than or equal to 0.1, among bacterial isolates. Strong biofilm-forming isolates are highlight in green. FIG. 9A shows biofilm formation among K. pneumoniae (KP) clinical isolates. FIG. 9B shows biofilm formation among E. cloacae (EB) clinical isolates. FIG. 9C shows biofilm formation of commercial strain of E. cloacae (ATTC 13047). FIG. 9D shows biofilm formation among E. coli (EC) clinical isolates. FIGS. 9E-9G show biofilm formation among P. mirabilis (PM) clinical isolates. Isolates were incubated for 24 h in triplicate (FIGS. 9A-9D) or 48 h in duplicate (FIGS. 9E-9G). results are presented in mean±−s.d.

FIG. 10 is a table showing bacterial-specific supplemented medias. PAO and K. pneumoniae utilized the standard minimal M9+ media described in the methods section of Example 4 under “Antisense-PNA treatment”.

FIG. 11A is a diagram showing a reaction scheme for dopamine polymerization into PD onto PVC tubes.

FIG. 11B is a set of images taken using an optical microscope at 11.2% magnification of PVC control tubes (left) and PD-Gent coated tubes (right) using 0.5 mg/mL dopamine during polymerization.

FIG. 12 is a graph showing the results pf gentamicin incorporated into composite coating on 25 cm PVC tube determined by LCMS. Error bars represent standard deviations of n=4.

FIG. 13 is a composite image of plates showing viability of PAO1 and 383-rahU::GM after incubation with PVC tubes coated with PD-Gent at varying dopamine concentrations (1-10 mg/mL).

FIG. 14A is a composite image showing CV staining of PD-only and PD-Gent tubes using PD concentrations of 0.25 and 0.50 mg/mL vs PAO1 and 383-rahU::GM.

FIG. 14B is a graph showing quantitation of biofilm formation on PVC tube-coated surface after PAO1 incubation for 24 hours. Coating was synthesized using 2 mg/mL gentamicin and increasing PD concentration. Error bars are representative of n=12.

FIG. 15 is a composite image showing PAO1 and 383-rahU::GM viability after incubating with PD-Gent coated tubes at PD concentrations 0.25-1.0 mg/mL.

FIG. 16 is a graph quantitating biofilm formation after PAO1 incubation with PD-Gent coated polystyrene 96-well plate. Error bars are representative of standard deviations of n=12.

FIG. 17A is a graph quantitating cell viability (measured in colony-forming units (CFU/mL) of PAO1 exposed to PD-only and PD-Gent 96-well plate coating. Error bars are representative of n=12.

FIG. 17B is a composite image of cell-viability assay for PD-Gent. coatings using PD concentrations 0.05-1.0 mg/mL.

FIG. 18A is a graph of biofilm quantification via crystal violet optical density on untreated and PD-Tobramycin coatings using PD concentration 0.25 mg/mL. Error bars are representative of n=18.

FIG. 18B is a composite image of PAO1 bacterial viability after exposure to coating for 24 h.

FIG. 19 is an image of a simulation of artificial bladder system with silicone urinary catheter. Urinary flow through the catheter was allowed to occur in a 30° C. oven while the bladder was allowed to hang outside the incubator.

FIG. 20 is an image of a plate showing serial dilution of biofilm from uncoated and PD-Gent coated catheter after 24 h in vitro study.

FIG. 21 is a graph showing quantitation of biofilm formation of 50% dilution in uncoated and PD-Gent coated Foley catheters after undergoing constant flow of PAO1-Artifical urine solution. Error bars are representative of n=3, ****: p-value≤0.0001.

FIG. 22 is a composite image of PAO1 cell viability of the initial bacteriuria solution, and solution after 2 and 5 h of continuous flow using PAO1-Artificial urine solution through PD-Gent coated catheter. The solutions were plated in 30 μL aliquots in two rows onto TSA plates immediately after collection from the catheter.

FIG. 23 is a table showing gentamicin attachment at PD concentrations of 0.05-1.0 mg/mL determined by LCMS.

FIG. 24 is a diagram showing the concept of an antibiotic releasing polymer composite coating deposited on an intraluminal surface of a catheter and its ability to prevent microbial growth.

FIGS. 25-31 are representations of additional sequence information for PNA implementations of the disclosure.

### BRIEF DESCRIPTION OF THE SEQUENCES

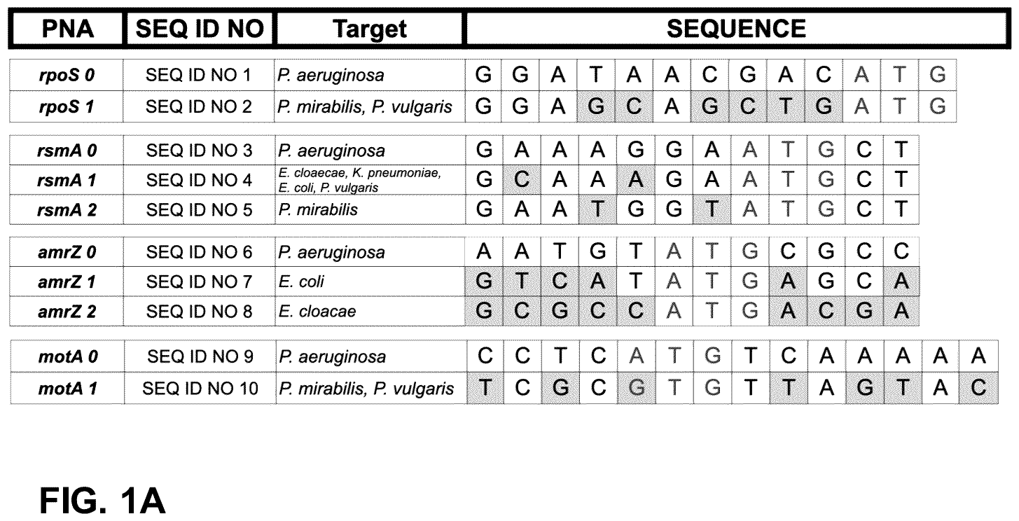

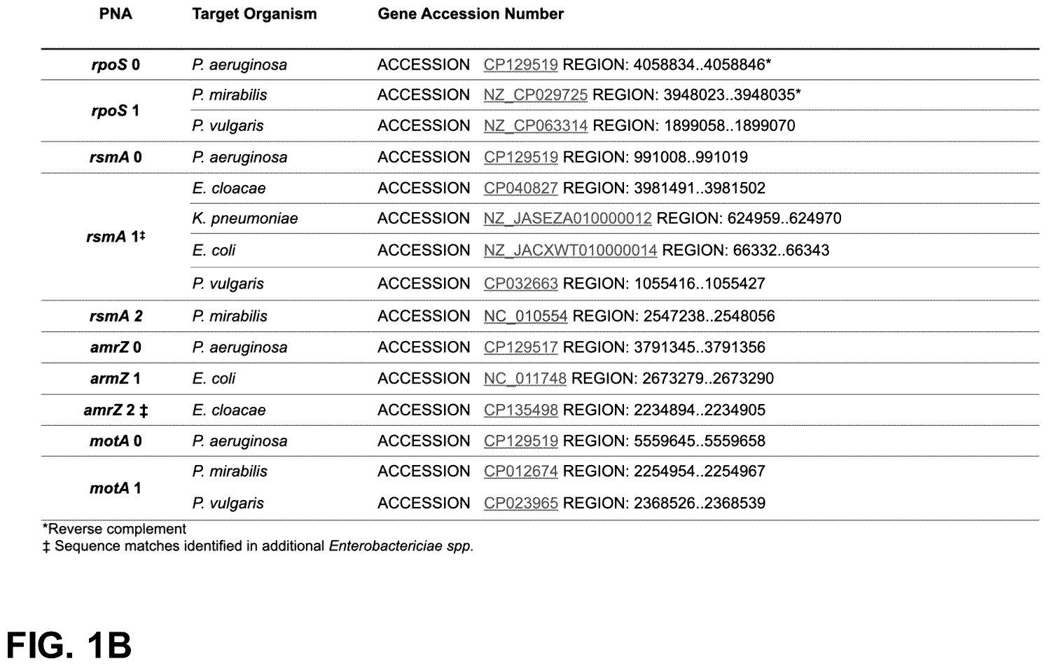

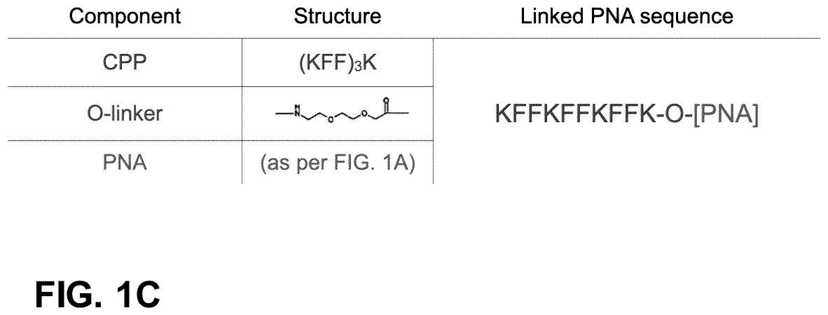

SEQ ID NOS: 1-10 are examples of peptide nucleic acids (PNAs) that can be used in various methods of the disclosure, such as incorporation into coatings or embedding into substrates. The peptide nucleic acids are synthetic constructs in which the deoxyribose phosphate backbone of DNA is replaced with a pseudo-peptide polymer to which nucleobases are joined. The sequences represent the specific order of nucleobases of each of the PNAs and are listed in the attached sequence listing and FIG. 1A.

SEQ ID NOS:11-13 are examples of cell penetrating peptides that can be linked to any of the PNAs of the disclosure. The cell penetrating peptides are synthetic constructs. The amino acid sequences are listed in the attached sequence listing and in the detailed description.

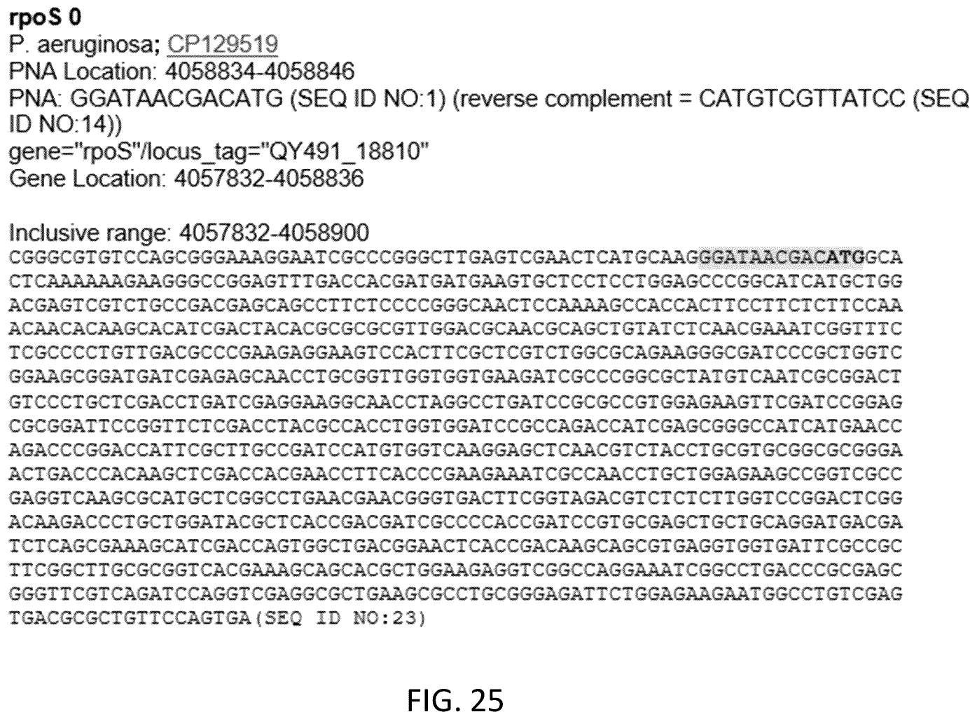

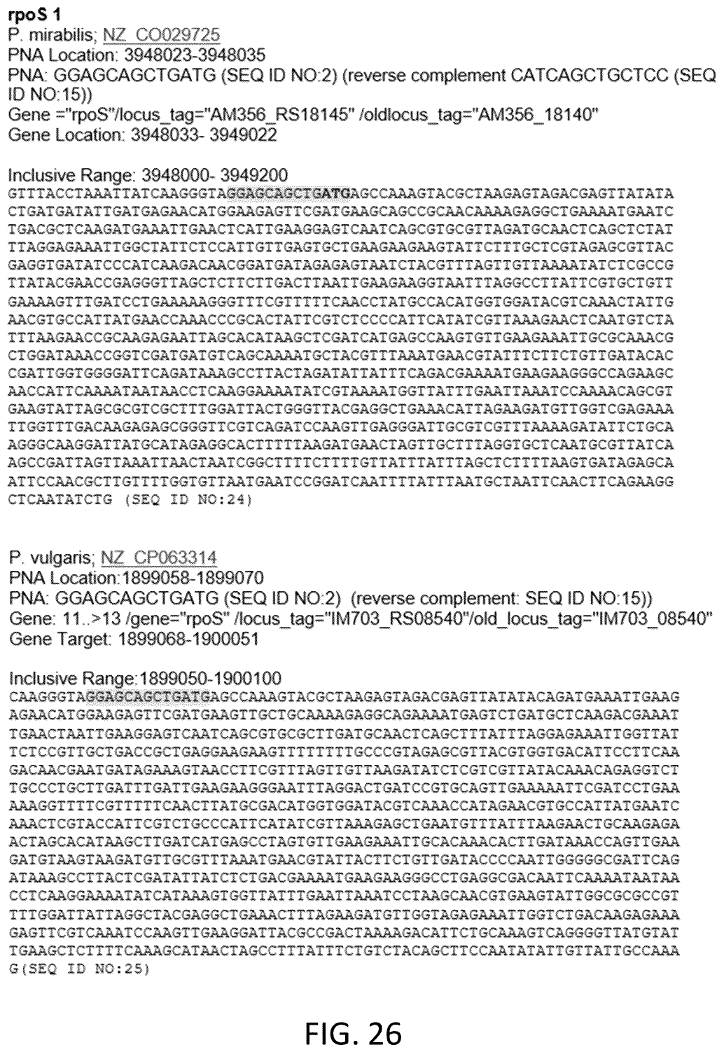

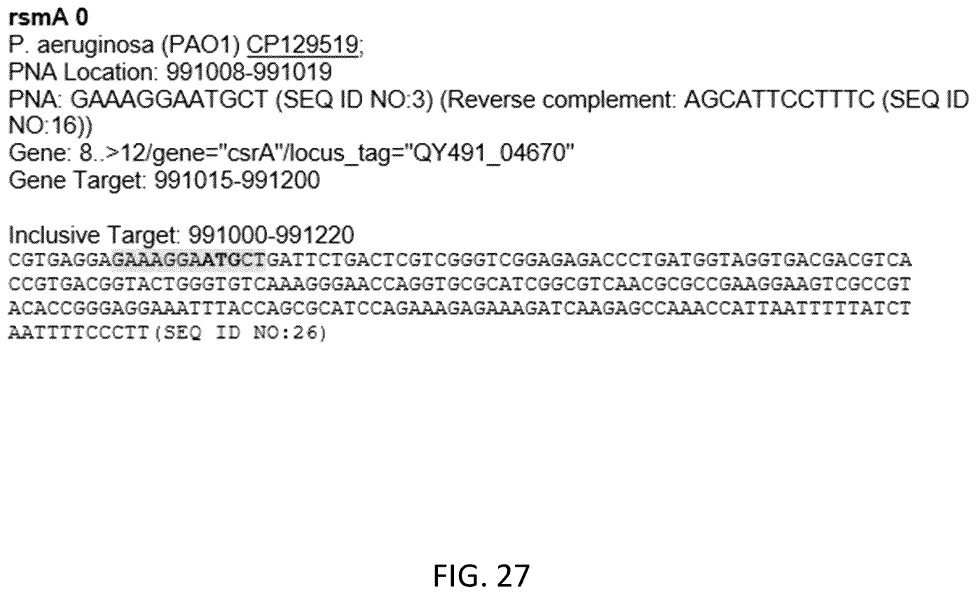

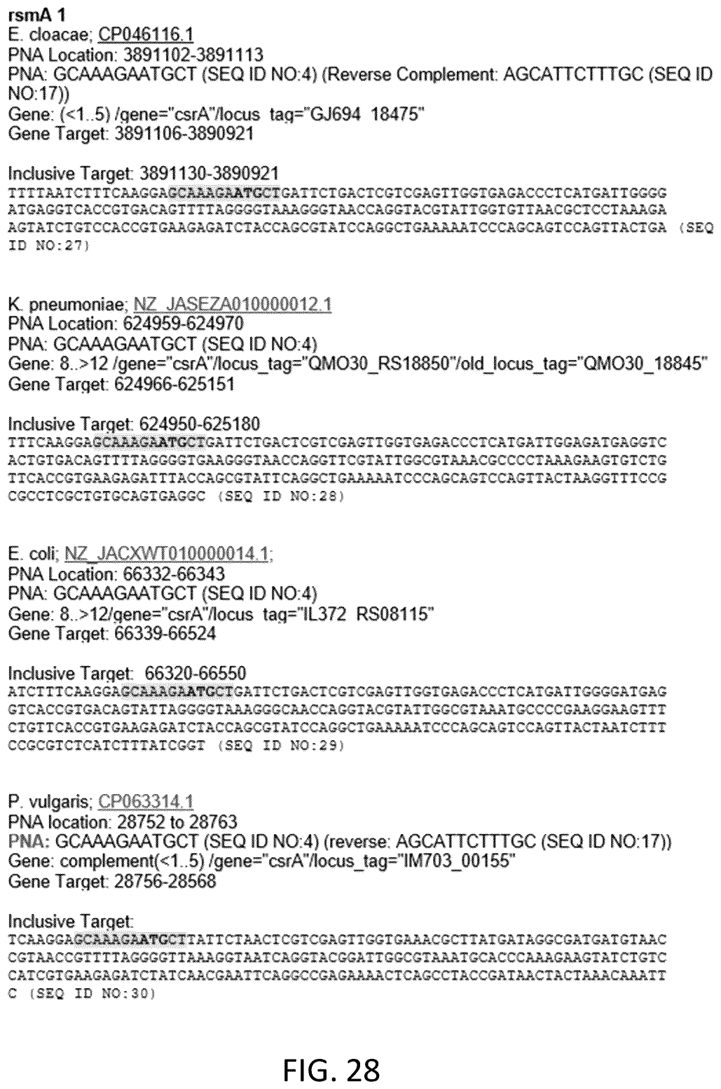

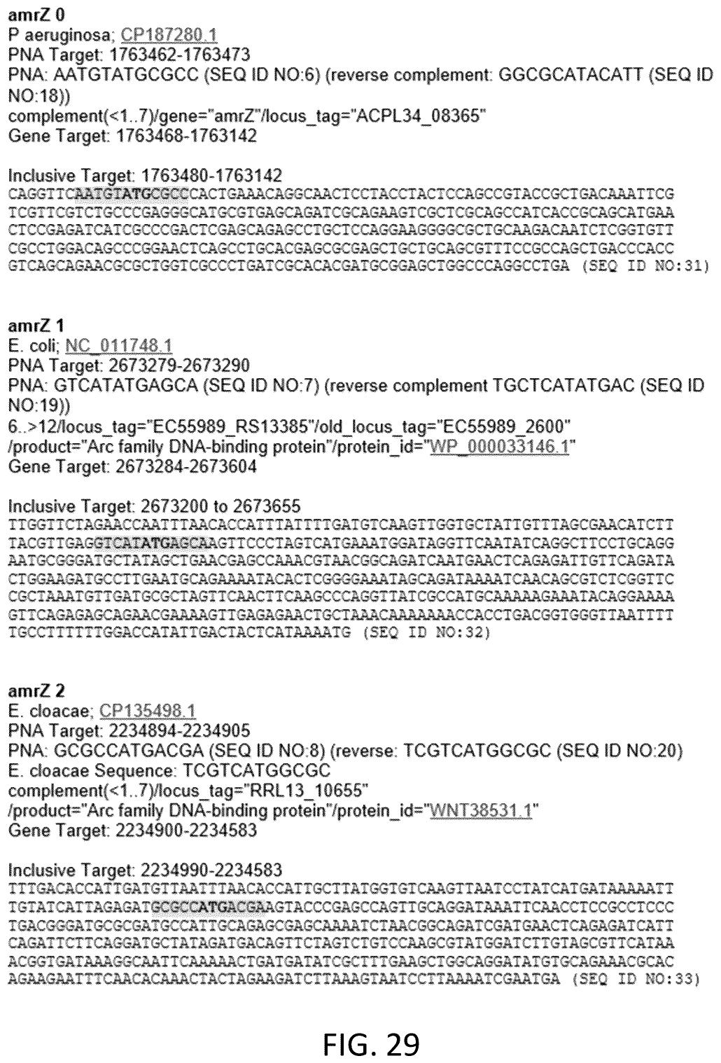

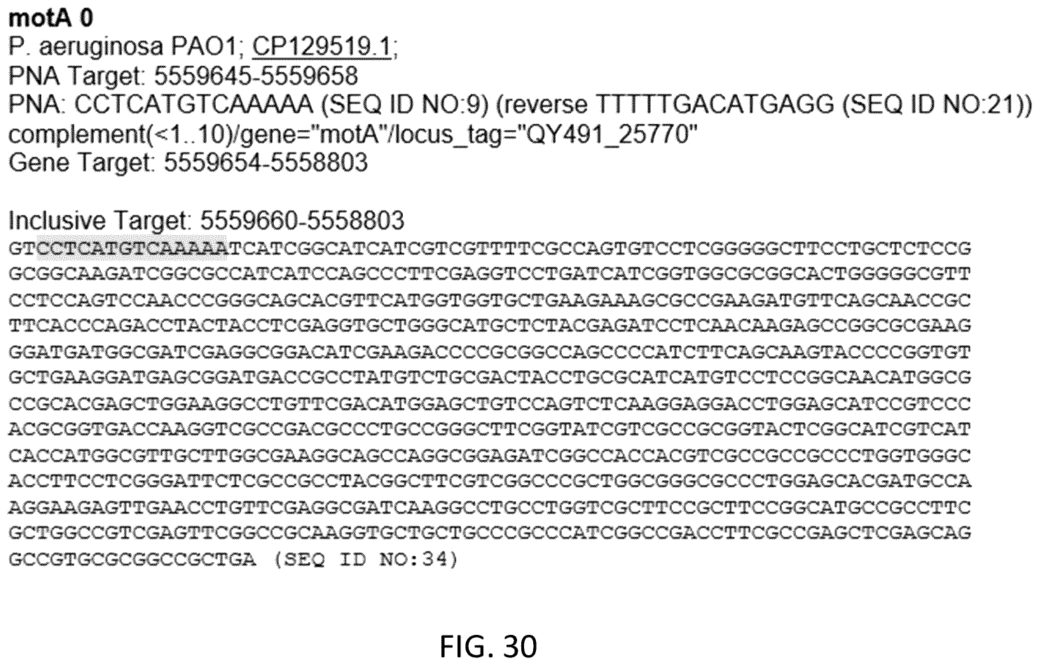

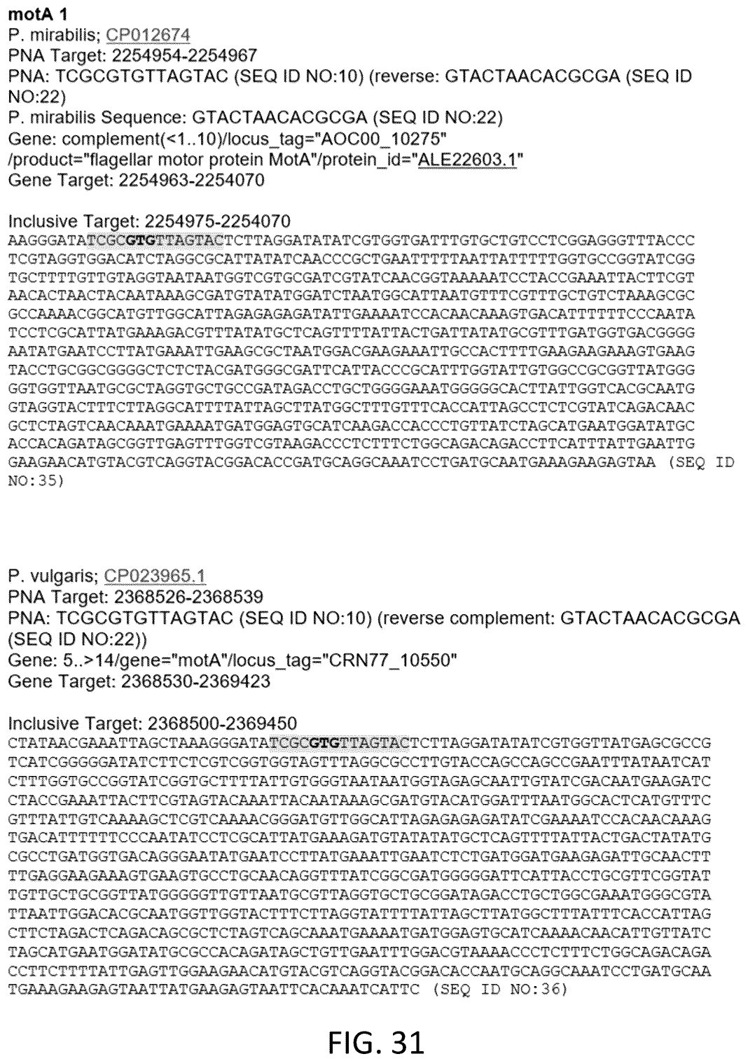

SEQ ID NOS:14-22 provide sequence information on reverse complement sequences of the PNAs. More information is provided in the Supplementary Sequence Information of the disclosure and in FIGS. 25-31.

SEQ ID NOS:23-36 provide sequence information on genomic target regions of the PNAs. More information is provided in the Supplementary Sequence Information of the disclosure and in FIGS. 25-31.

### DETAILED DESCRIPTION

Reference will now be made in detail to various illustrative implementations. It is to be understood that the following discussion of the implementations is not intended to be limiting.

### Abbreviations

LB: luria broth miller

TSA: tryptic soy agar

TSB: tryptic soy broth

M9: minimal salt medium

CV: crystal violet

PNA: Peptide nucleic acids

The present disclosure relates to peptide nucleic acids (PNAs) (e.g., one or more nucleobase sequence chosen from SEQ ID NOS:1-10 (or any reverse, reverse complementary, or complementary sequence thereof), or a nucleobase sequence that has at least about 80%, 85%, 90%, 95%, 96%, 97%, 98% and 99% sequence identity to a nucleobase sequence chosen from SEQ ID NOS:1-10 (or any reverse, reverse complementary, or complementary sequence thereof). PNAs are synthetic polymers in which the deoxyribose phosphate backbone of DNA is replaced with a pseudo-peptide polymer to which nucleobases are joined (Nielsen et al., 1991; Pellestor and Paulasova, 2004). PNAs are powerful tools for molecular genetics and cytogenetics (Pellestor and Paulasova, 2004). PNAs are resistant to enzymatic degradation, and can hybridize to complementary DNA with high specificity and affinity. The present disclosure encompasses PNAs that target certain regulatory or other critical gene sequences, such as rpoS, rsmA, amrZ, and motA, of bacteria that are known to form biofilms. The PNAs are designed to target the regulatory gene or other critical sequences through antisense hybridization. As a result of hybridization to their intended target, the PNAs inhibit the growth of microbes involved in the formation of biofilms. As such, applying the PNAs as a solution or other liquid-based form to surfaces effectively reduces or eliminates biofilm formation on such surfaces. The surfaces to which the PNAs are applied can include those in medical settings or industrial settings, such as various equipment. Reducing the formation of biofilms on such surfaces increases the operational efficiency and lifetime durability of such equipment and prevents poor patient outcomes, including nosocomial infections.

Embodiments of the PNAs are designed to bind complementary DNA sequences in bacterial organisms such as P. aeruginosa, E.coli, K. pneumoniae, P. mirabilis, P. vulgaris, and E. cloacae, and thereby inhibit gene transcription. Each bacterial organism which the PNAs are designed to inhibit are referred to herein as a target organism. Embodiments of PNAs, their corresponding SEQ ID NO., their target organism, and the nucleobase sequence of the PNAs are shown in FIG. 1A. The PNAs collectively target a wide range of microbial target organisms and can be implemented in one or more combinations and/or in other forms such as reverse, reverse complementary, or complementary sequences of the nucleobase sequences of FIG. 1A.

Implementations of the PNAs can be used for therapeutic applications, medical or industrial hygiene applications, or diagnostic applications, to name a few. PNAs that hybridize to the template strand of disclosed regulatory and motility genes during transcription inhibit growth and biofilm formation. These PNAs are exemplified in SEQ ID NOS: 1-10 and variations of SEQ ID NOS:1-10, such as those with at least about 80%, 85%, 90%, 95%, 96%, 97%, 98% and 99% sequence identity to SEQ ID NOS:1-10 that hybridize to the template strand of their intended target gene. Such PNAs may also be useful as probes for research, detection of contaminated surfaces, or diagnosis of infections. Other PNAs that can be used as probes include variations such as sequences which are complementary, reverse, or reverse complementary to SEQ ID NOS:1-10 that hybridize to a complementary nucleic acid in an intended target organism. The PNA probes can incorporate or be conjugated or linked to a fluorophore, a radioisotope, a reporter, or similar signaling molecule capable of being detected by appropriate instrumentation and indicate binding of the PNA probe to a genomic region of the target organism and can indicate or identify multiple species within biofilms or detect contamination of an abiotic or biotic surface with the target organism. The PNA probes can also incorporate, be linked to, or conjugated with companion molecules that inhibit nucleic acid function (or are otherwise toxic to nucleic acids) such as other transcriptional inhibitors, replication inhibitors, mutagens, nucleases and the like, and in this way act to disrupt nucleic acid processes and thereby inhibit growth and biofilm formation. As such, it is contemplated that any of the PNA probes that hybridize to an intended genomic target region can potentially have therapeutic, hygienic, or diagnostic or detection applications or capabilities, depending on the properties of the companion molecule that the PNA probe incorporates, or is conjugated with or linked to.

A first set of PNAs shown in FIG. 1A target regulatory and motility gene sequences found in P. aeruginosa including rsmA (Genbank Accession AAG04294, also known as csrA), amrZ (Genbank Accession AAG08856), rpoS (Genbank Accession AAG07010), and motA (Genbank Accession AAG08339). A cocktail of PNAs is termed “cPNAs” and includes rsmA 0, amrZ 0, and rpoS 0. The motA 0 PNA can be used individually or in combination with the cPNAs. Each PNA sequence is 12 to 14 nucleobases and inclusive of a start codon; they were each tested for specificity, palindromic sequences and hairpin formation. In embodiments, any one or more of rsmA 0, amrZ 0, rpoS 0 and motA 0 can be used, for example to target P. aeruginosa. For example, the cPNAs can comprise rsmA 0 and amrZ 0, or amrZ 0 and rpoS 0, or rsmA 0 and rpoS 0. In embodiments, the PNA sequences can comprise from 12-14 base pairs as shown. Variations of PNA sequences comprising 11-15 base pairs or 10-16 base pairs which hybridize to target regulatory and motility gene sequences are also contemplated, including any reverse, reverse complementary, or complementary sequence of the PNAs shown in FIG. 1A.

A second group of PNAs shown in FIG. 1A, including rpoS 1, rsmA 1 and rsmA 2,amrZ 1 and amrZ 2, motA 1 are termed wide range or wrPNAs, and are designed to target Gram-negative bacteria with similar (or relatively conserved) regulatory genes originally identified in P. aeruginosa. Their nucleobase sequences were developed by searching the NCBI database for such conserved regulatory genes and were designed and adjusted to account for interspecies variation.

As described in the Examples, the wrPNAs were tested in four Gram-negative bacteria, including K. pneumoniae, E. cloacae, E. coli, and P. mirabilis. In embodiments, any one or more of rpoS 1, rsmA 1, rsmA 2, amrZ 1, amrZ 2 and/or motA I can be used, for example, to treat/target any one or more of K. pneumoniae, E. cloacae, E. coli and/or P. mirabilis and P. vulgaris. In embodiments, the PNAs can comprise rpoS 1 and rsmA 1, or rpoS 1 and rsmA 2, or rpoS 1 and amrZ 1, or rpoS 1 and amrZ 2, or rpoS 1 and motA 1, or rsmA 1 and rsmA 2, or rsmA 1 and amrZ 1, or rsmA 1 and amrZ 2, or rsmA 1 and motA 1, or rsmA 2 and amrZ 1, or rsmA 2 and amrZ 2, or rsmA 2 and motA 1, or amrZ 1 and amrZ 2, or amrZ 1 and motA 1, or amrZ 2 and motA 1, and further combinations. In embodiments, the PNA sequences for the wrPNAs can comprise from 12-14 base pairs as shown. Variations of wrPNA sequences comprising 11-15 base pairs or 10-16 base pairs that hybridize to conserved regulatory genes in Gram negative bacteria are also contemplated, including any reverse, reverse complementary, or complementary sequence of the PNAs shown in FIG. 1A.

The variations of nucleobase sequences of the wrPNAs in comparison to those targeting P. aeruginosa are depicted in FIG. 1A with changes in sequence indicated with shading. Each wrPNA shown in FIG. 1A includes a start codon [ATG or GTG] and comprises 12-14 bases; sequences were further assessed for specificity, palindromic formation and hairpin formation.

Accession numbers and genomic regions, for gene target examples are provided in FIG. 1B. The PNAs selectively and specifically hybridize to the bacterial genomes indicated by the accession numbers at the target regions (or target genes, target sites, or target sequences) delineated by the genomic coordinates through antisense hybridization (through Watson-Crick base pairing or binding through the nucleobase portion of the PNAs) to complementary DNA sequences indicated by the genomic coordinates. As a result of such hybridization, the start codon of the bacterial gene is blocked and transcription is impaired. Although PNAs of 12 to 14 nucleobases (or their target complementary region) are shown, variations of the PNAs shown in FIG. 1A and 1B, which are of shorter (e.g., 10 or 11 nucleobases) or of longer (e.g., 15 or 16 nucleobases) length and which complementary DNA sequences include the start codon are also contemplated. Gene targets can include any reverse, reverse complementary, or complementary sequence to those examples provided in FIG. 1B in some implementations. The variations can be assessed or tested for specificity, palindromic formation and hairpin formation, and those properties can be compared to those of the PNAs shown in FIGS. 1A and 1B. As such, the PNAs shown in FIGS. 1A and 1B can serve as models by which variations that deviate from their length and/or sequence are designed but share substantially similar properties, including antisense hybridization to the gene targets provided in FIG. 1B. Such assessment or testing can occur in silico or through in vitro testing. Specificity can be assessed through searches of publicly available sequence databases such as NCBI (Blast). Tools for assessing palindromic formation and hairpin formation include those provided on web-based applications such as Integrative DNA Technologies OligoAnalyzer™ Tool.

The PNAs of the disclosure can be linked to a cell penetrating peptide (CPP) to increase uptake into their target organisms. In one embodiment, the PNAs are linked to a CPP with amino acid sequence KFFKFFKFFK (SEQ ID NO: 11). The PNAs and CPPs can be bound together with a linker such as an organic molecule that improves solubility (FIG. 1C).

The PNAs can be linked to other molecules such as a reporter. Non-limiting examples of reporters are alkaline phosphatase, galactosidase, peroxidase, luciferase and green fluorescent protein (GFP). The PNAs can also be linked to an epitope tag such as c-myc. The PNAs can also be labeled with various fluorophores such as by incorporating fluorophore-coupled nucleobases. Such linked or labeled PNAs can be used as probes for identifying multiple species within biofilms.

The PNAs can be implemented in alternative ways that fall within the scope of the disclosure. Such modifications include the substitution of other CPPs linked to the PNA oligomers. Approximately 1850 CPPs have been identified and their information curated online (see Agrawal P, Bhalla S, Usmani SS, Singh S, Chaudhary K, Raghava GP, Gautam A (2015) CPPsite 2.0: a repository of experimentally validated cell penetrating peptides. Nucleic Acids Research doi: 10.1093/nar/gkv1266 and Gautam A, Singh H, Tyagi A, Chaudhary K, Kumar R, Kapoor P, Raghava GP (2012). CPPsite: a curated database of cell penetrating peptides. Database (Oxford). March 7;2012: bas015). Additionally, Hyang-Mi Lee and others disclose methods for constructing and screening a library of CPPs enabling the design of new and efficient CPPs for bacterial biotechnology and other applications (see Lee, HM., Ren, J., Tran, K.M. et al. Identification of efficient prokaryotic cell-penetrating peptides with applications in bacterial biotechnology. Commun Biol 4, 205 (2021). Some examples of alternative CPPs that can be substituted include RXRRXRRXRRXRXX (SEQ ID NO:12), where the last X can be beta-alanine and the remaining X's can vary from 2 to 8 carbons such as glycine, 4-aminobutyric acid, 6-aminohexanoic acid, or 8-aminocaprylic acid (Barkowsky et al., 2019, Abes et al., 2008) and GWTLNSAGYLLGKINLKALAALAKKIL (SEQ ID NO:13) (Barkowsky et al., 2019).

The PNAs can also vary with respect to the sequence of their oligomers according to some implementations. The PNAs can include oligomers that vary by one or more nucleobase shown in the sequences of SEQ ID NOS:1-10 or any reverse, reverse complementary, or complementary sequence thereof. As such, the PNAs can include oligomers that have less than 100% sequence identity to SEQ ID NOS:1-10 (or any reverse, reverse complementary, or complementary sequence thereof) but retain their antisense function in inhibiting their intended target organism, including P. aeruginosa, P. mirabilis, P. vulgaris, E. cloacae, E.coli, and K. pneumoniae. Such PNAs include oligomers with at least about 80%, 85%, 90%, 95%, 96%, 97%, 98% and 99% sequence identity to an oligomer including one or more nucleobase sequences of SEQ ID NOS:1-10 or any reverse, reverse complementary, or complementary sequence thereof. As used herein, “identity” is a measure of the identity of nucleobase sequences, nucleotide sequences, or amino acid sequences compared to a reference nucleobase, nucleotide or amino acid sequence. In general, the sequences are aligned so that the highest order match is obtained. “Identity” per se has an art-recognized meaning and can be calculated using published techniques. Also included are PNA oligomers 10 to 16 nucleobases in length, such as 11 to 15 nucleobases, or 12 to 14 nucleobases, capable of hybridizing through antisense hybridization to a complementary base sequence within, represented by, or inclusive of a genomic region shown in FIG. 1B. In this manner, the PNA oligomers are designed to target the genomic regions represented in FIG. 1B. The oligomers can be designed by methods of the disclosure to retain their antisense activity and specificity for the bacteria shown in FIGS. 1A and 1B as well as other Gram-negative, Gram-positive and atypical bacteria by searching for homologous genes related to biofilm and regulatory function.

### Methods of Manufacture

Following their design as described herein, the PNAs were synthesized by PNA Bio (Newbury Park, CA). The designated PNAs were linked to a cell penetrating peptide (CPP) and an O-linker to enhance solubility, and shipped as a dry powder for reconstitution in the laboratory.

Synthesis of PNAs can be achieved through first synthesizing monomeric building blocks composed of an amino acid or amino acid analog backbone portion and a nucleobase portion. The monomeric building blocks can then be attached together through solid phase synthesis techniques such as those described in U.S. Pat. No. 6,451,968B1, herein incorporated by reference in its entirety, to form a desired sequence of nucleobases such as those set forth in SEQ ID NOS:1-10 (or any reverse, reverse complementary, or complementary sequence thereof) or those at least 80% identical to SEQ ID NOS: 1-10 (or any reverse, reverse complementary, or complementary sequence thereof) or other sequences of nucleobases designed to target the genomic regions represented in FIG. 1B or any reverse, reverse complementary, or complementary sequence thereof. Other methodologies or combinations thereof, such as combination with the solid-phase technique, are described in U.S. Pat. No. 7,378,485B2, herein incorporated by reference in its entirety.

### Methods of Use

One implementation of a method of embodiments of the invention includes applying a coating to a medical device, the coating including one or more PNAs that include one or more oligomer having a nucleobase sequence chosen from or at least 80% identical to SEQ ID NOS: 1-10 or any reverse, reverse complementary, or complementary sequence thereof or of the PNAs shown in FIG. 1A or other oligomers designed to target the genomic regions represented in FIG. 1B, or any reverse, reverse complementary, or complementary sequence of these genomic regions. The medical device can be for example a urinary catheter; a central line; a port; prosthetic joints and orthopedic hardware; stents or drains used in gastrointestinal, pancreatic, hepatobiliary, or genitourinary locations; cardiac devices, including pacemakers, implantable cardio-defibrillators, prosthetic valves, or stents; an endotracheal or tracheostomy tube; a medical instrument such as a mesh, sterile dressing, probe or needle, or any other medical device such as those described in this disclosure. A variation of this implementation includes applying a gel, flush or solution that includes one or more of the described PNAs to such medical device.

Another implementation of a method includes treating a patient with one or more PNAs that include one or more oligomer having a nucleobase sequence chosen from or at least 80% identical to SEQ ID NOS: 1-10, including any reverse, reverse complementary, or complementary sequence thereof or of the PNAs shown in FIG. 1A or other oligomers designed to target the genomic regions represented in FIG. 1B or reverse, reverse complementary, or complementary sequences of those regions. The patient can be treated topically with the PNAs at sites prone to infection such as burn wounds, diabetic foot wounds, mucosal surfaces such as the urinary tract or bladder, a body cavity, or the ear canal. The patient can also be treated by intrapulmonary application of aerosolized PNAs for treating pneumonia, cystic fibrosis, or other chronic lung disease. The methods of treatment can include treatment of various infections with biofilm involvement, or prophylactic treatments of sites prone to such infections.

Another implementation of a method of embodiments of the invention includes applying a coating or solution that includes one or more PNAs that include one or more oligomer having a sequence chosen from or at least 80% identical to SEQ ID NOS: 1-10, including any reverse, reverse complementary, or complementary sequence thereof or of the PNAs shown in FIG. 1A or other oligomers designed to target the genomic regions represented in FIG. 1B or reverse, reverse complementary, or complementary sequences of those regions, to an industrial device such as HVAC systems, air filters, water purification systems, industrial tubing or pipes, ships, and aquatic equipment, or any other industrial device such as those described in this disclosure.

Another implementation of a method of embodiments of the invention includes applying, coating, or embedding one or more PNAs that include one or more oligomer having a sequence chosen from or at least 80% identical to SEQ ID NOS: 1-10, including any reverse, reverse complementary, or complementary sequence thereof or of the PNAs shown in FIG. 1A or other oligomers designed to target the genomic regions represented in FIG. 1B or reverse, reverse complementary, or complementary sequences of those regions, to one or more surfaces within a healthcare setting including high touch surfaces/countertops; tables; lavatory surfaces; equipment such as stethoscopes, ultrasound equipment, monitors, ventilators, extracorporeal life support machines; patient beds and bedding; wheelchairs; stretchers; gloves or other garments; sponges, wipes, pads, or mops; packaging materials of sterile medical or hospital supplies; or sutures or wound dressings. For some of these, such as sponges or wound dressings, embedding or impregnating the one or more PNAs into the substrate of the object would be preferred.

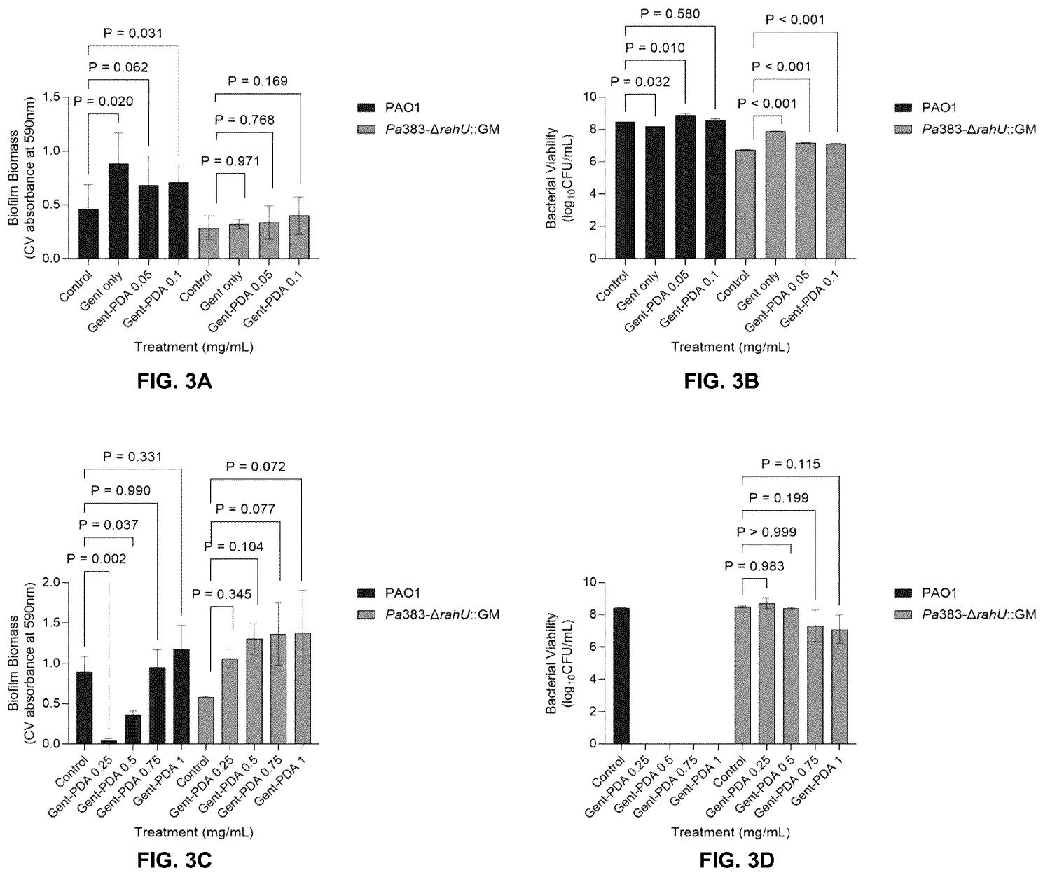

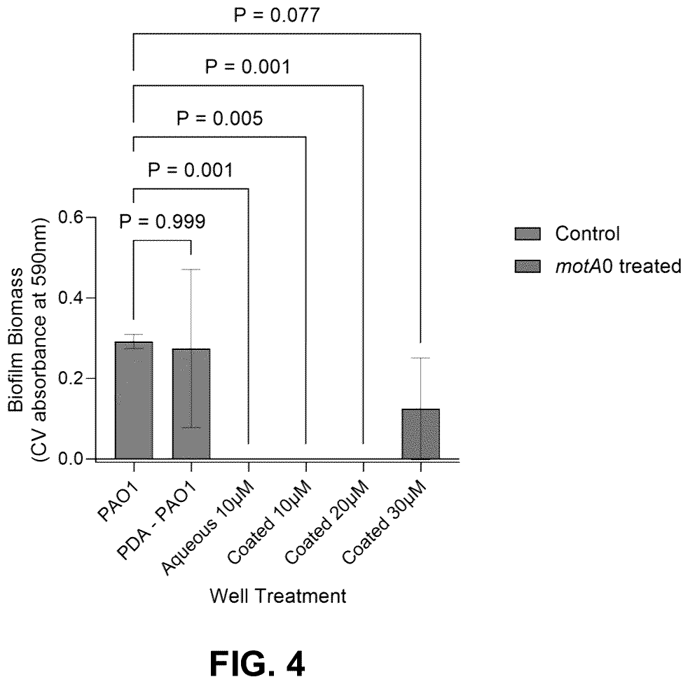

The above implementations can be combined with antibiotic applications or other treatments to enhance bactericidal effects. For example, polydopamine (PDA) can be used as a basis for antibiofilm coating. As detailed in the Examples, gentamicin combined with PDA coating (0.25 and 0.5 mg/mL) inhibited P. aeruginosa (PAO1) biofilm formation and cell viability after 24 hrs incubation (FIGS. 3C and 3D). When motA PNA was combined with 0.25 mg/ml PDA it was shown to inhibit P. aeruginosa (PAO1) biofilm formation after 24 hours incubation (FIG. 4). PDA can be used to coat one or more PNAs of the disclosure (e.g., one or more nucleobase sequence chosen from SEQ ID NOS:1-10 (or any reverse, reverse complementary, or complementary sequence thereof), or a nucleobase sequence that has at least about 80%, 85%, 90%, 95%, 96%, 97%, 98% and 99% sequence identity to a nucleobase sequence chosen from SEQ ID NOS:1-10, or any reverse, reverse complementary, or complementary sequence thereof) and antibiotics or can be combined in treatments and applications described herein. Other antibiotics that can be applied can be chosen from classes of antibiotics such as aminoglycosides, carbapenems, cephalosporins, fluoroquinolones, macrolides, monobactams, oxazolidinones, penicillins, rifamycins, sulfonamides, and tetracyclines. One particular implementation of an antibiotic is gentamicin. Other agents that can be included in the antibiofilm coating can include other antimicrobial agents such as antiseptics, antiviral agents, antifungal agents, or a disinfectants. Illustrative examples of coating procedures of medical devices with an antimicrobial agent are described in U.S. Patent No. U.S. Pat. No. 10,589,003, herein incorporated by reference in its entirety. In general, these methods involve contacting the surface to be coated with a composition that includes an antimicrobial agent and a solvent and curing the surface by applying heat. Such methods can be performed simultaneously with or separately from the following methods of applying, coating, or embedding PNAs onto abiotic surfaces.

Methods of applying, coating, or embedding the one or more PNAs on one or more abiotic surfaces can include forming a solution or suspension of the one or more PNAs, covering the surfaces with the solution or suspension (e.g., by dip coating, spray coating using a spray applicator, brush coating, roll coating, or other method) and drying the surfaces such as through applying low heat to the surfaces for a period of time to evaporate the liquid or solvent to leave the one or more PNAs adhered to the abiotic surfaces. Coating chemically adheres or bonds one or more layer of PNAs, antibiotics, or other agents to the abiotic surface oriented substantially parallel to the abiotic surface, while embedding (or infiltrating or impregnating) exposes the abiotic surface to the solution or suspension for a period of time to allow for uptake of the solution or suspension into a substrate. Loss of the liquid or solvent from evaporation leaves the PNAs adhered to the abiotic surface in the case of coating or trapped in the substrate in the case of embedding. The coating can include material covering any portion of an abiotic surface and can be configured as one or more coating layers. The coating can include only the PNAs and other agents (e.g. antibiotics) added to the solution or suspension, or can include material capable of forming a carrier such as carriers described in the following compositions. For example, monomers capable of forming a polymeric carrier can be added to the solution or suspension of PNAs with other agents such as catalysts such that they polymerize to produce a coating of polymer on the abiotic surface that is impregnated with the PNAs. The coating can have a substantially constant or a varied thickness and composition. The thickness of the coating may be selected to provide a desired rate of release. The coating preferably has a thickness of about 0.1 μm to about 100 μm, including about 0.2 μm to about 80 μm, or between about 0.4 μm to about 40, 50 or 60 μm. Properties of the coating can be adjusted by altering the concentration of PNAs or antibiotics or other agents in the solution or suspension, or adjusting the atomization pressure of the spray applicator to provide a desired coating morphology and/or spray plume droplet size.

The coating can be applied to all of the surfaces of an object (such as a medical device, industrial device, or surface in a healthcare setting), or only select surfaces. For example, medical tubing or other conduits such as industrial tubing or pipes would be coating intraluminally in some implementations but not extraluminally. The same strategy would be applied to other surfaces prone to contamination, such that the coating is selectively applied to those surfaces leaving other surfaces (i.e., those which are not prone to biofilm formation, such as dry surfaces) uncoated.

Various methodologies can be used to incorporate the PNAs into polydopamine coatings. Examples of polydopamine coating methodologies include those described in Chinese Patent No. CN108815552B, Chinese Patent Application No. CN112295022A, Russian Patent No. RU2698315C2, International Patent Application Publication No. WO2018191681A1, U.S, Patent Nos. 10,016,499B2 and 9,770,418B2, and U.S. Patent Publication No. US20230024332A1, as well as in the literature (Lim, K, et al. 2015; Singh, I., et al, 2020; Batul, R, et al., 2023; Michaelicha, A. et al., 2021), each patent, patent application publication, or literature article herein incorporated by reference in its entirety.

In addition or alternatively to antibiotics, the disclosure contemplates incorporation of other agents within the coating according to particular applications. For medical devices, therapeutics such as antithrombotic agents (e.g., antiplatelets, anticoagulants, fibrinolytic agents) can be added to the coating such as for coatings for stents or medical tubing.

### Compositions

One or more of the PNAs can be formulated in a pharmaceutical carrier, such as a solution, suspension, emulsion, ointment, cream, or gel. The PNAs can be formulated in various solutions with sterile solvents. The sterile solvents can include distilled water, quick-drying, volatile solvents such as methanol, ethanol, or dichloromethane for coating applications, and, for health applications such as irrigating wounds, isotonic diluents such as 0.9% NaCl, 5% dextrose in water, and Ringer's lactate solution. The diluents can include other ingredients such as buffers and preservatives. Aerosol sprays suitable for inhalation by the patient are also contemplated. The PNAs can also be formulated in various ointment bases such as a variety of hydrophobic and hydrophilic ointment bases that are available. Hydrophobic bases are composed of hydrocarbons (oleaginous compounds) and include white ointment, white petroleum, mineral oil, wax and other paraffins, and VASELINE®. Hydrophilic bases include anhydrous absorption bases such as hydrophilic petrolatum, anhydrous lanolin, POLYSORB®, AQUAPHOR®, and AQUABASE®; these will absorb significant amounts of water. Water-in-oil emulsion ointment bases are hydrous bases such as hydrous lanolin, cold cream, EUCERIN®, HYDROCREAM®, rose water ointment, and NIVEA®. Other examples of hydrophilic absorption bases include oil-in-water emulsion bases, water-soluble bases, and gels. Formulas for these ointment bases have been described in the pharmacy literature (see De Villiers, Melgardt. (2009). Ointment Bases. In book: A Practical Guide to Contemporary Pharmacy Practice (pp.277-290) Edition: 3 Chapter: 23 Publisher: Lippincott Williams & Wilkins Editors: Judith E Thompson; and Adejare A, ed. (2020), Remington: The Science and Practice of Pharmacy, 23rd Ed. (Academic Press, Cambridge, MA) Publisher: Academic Press, each incorporated herein by reference). The PNAs can be formulated in various combinations with one or more antibiotics.

In some implementations, the pharmaceutical carrier is or includes one or more polymer. The one or more polymer is capable of being applied to various surfaces as a solution or suspension and allowed to dry or allowed to form in situ by combining monomers with a catalyst in the applied solution or suspension. The one or more polymer is chosen from one or more of a polyester, a polyurethane, a poly(meth) acrylate, a polysaccharide, a polyamide, a polynorbornene, a polycarbonate, a poly(meth) acrylamide, a polyoxazoline, a poly(ethylene oxide), a polyaziridine, or a polysiloxane.

In some implementations, the pharmaceutical carrier is or includes a polymer of dopamine (polydopamine or PDA), which can be present in the carrier in the range of 5% to 100% w/w.

Such pharmaceutical carriers such as polymeric carriers can be used in the methods of use above, such as applying a coating to a surface of a medical device, an industrial device, or one or more surfaces within a healthcare setting. The coating with one or more of the PNAs can be applied evenly on such surfaces by spray coating or other method to provide a uniform distribution of PNAs and can include one or more antibiotics such as polydopamine or other antibiotics. The coating can also be applied by dissolving the PNAs in volatile solvents such as methanol, ethanol, or dichloromethane in a spray or other application solution. The concentrations of PNAs in such coatings can be in the range of 10 μM to 5 M, such as 15 μM to 100 μM, or 50 μM to 200 μM, or 150 μM to 500 μM, or 300 μM to 1 mM, or 750 μM to 100 mM, or 50 mM to 450 mM, or 200 mM to 1 M, or 500 mM to 10 M, or 1 M to 3 M, or 2 M to 4 M, or 1-5 M.

The pharmaceutical carrier can provide for controlled release of the one or more PNAs or other agents such as antibiotics or other therapeutic agents. The controlled release of the PNAs or other agents can be constant or can vary with time. The controlled release can be determined from an elution profile of the one or more PNAs or other agents, which shows the measured rate at which the PNAs or other agents are removed from a coated abiotic surface in an elution medium as a function of time. Various pharmaceutical carriers may differ in their controlled release properties, with some providing longer duration or sustained release, and these can be determined from such elution profiles.

### Kits

The PNAs can be provided as components of kits made available to the consumer. The kits can include PNAs formulated individually or in various combinations as a dry powder in a sterile vessel such as a vial, bottle, or drum (depending on the application and volume of solution or suspension required) to which a solvent can be added for reconstitution. The vessel can include a cap through which a sterile needle or tube can be inserted for adding the solvent. The solvent can be included as part of the kit or provided separately and can include any aqueous or organic solvent in which the PNAs are soluble. The kits can include written instructions on the vessel or provided separately for adding an appropriate type and amount of solvent. The kits can also include equipment such as spray applicators or brushes for applying the reconstituted PNAs.

### Medical Devices

Non-limiting examples of medical devices of the disclosure include wound dressings or transdermal patches impregnated with one or more of the PNAs, or medical grade tubing, dialysis equipment, IV solution bags, infusion pumps, infusion ports, catheters, central lines, ports, drains, endotracheal tubes, tracheotomy tubes, ureteral stents, biliary stents, ventriculostomy catheters, chest tubes, gastric tubes, intestinal tubes, nephrostomy tubes, prosthetic joints and other orthopedic devices, vascular stents, stent grafts, vascular grafts, guide wires, balloons, sutures, staples, filters (e.g. vena cava filters), cerebral aneurysm filler coils, mesh, pacemakers or other cardiac devices, prosthetic valves, anastomosis devices, vertebral disks, bone pins, suture anchors, hemostatic barriers, clamps, screws, plates, clips, slings, vascular implants, tissue adhesives and sealants, tissue scaffolds, myocardial plugs, pacemaker leads, abdominal aortic aneurysm (AAA) grafts, embolic coils, various types of dressings, bone substitutes, intraluminal devices, vascular supports, or other known bio-compatible devices or medical instruments such as a probe or needle coated with the PNAs, as well as other instruments meant to be inserted into a human or animal body or body part. The PNAs can be impregnated or coated in combination with antibiotics or other therapeutic agents.

### Industrial Devices

Industrial devices of the disclosure include any device or system where water provides an opportunity for growth of biofilm-forming bacteria. These include HVAC systems, air filters, water purification systems, industrial tubing or pipes, ships, industrial or municipal waste water distribution systems, industrial potable water systems, industrial or power plant cooling systems (e.g., cooling tower, nuclear reactor cooling water), pulp and paper mills, industrial food and dairy processing facilities, pharmaceutical or chemical manufacturing systems, cosmetics manufacturing systems, petrochemical pipelines, irrigation systems, and aquatic equipment coated with the PNAs or combined with one or more antibiotics or other agents. Such systems can include various equipment such as pumps, reactors, tanks, vessels, mixers, agitators, and the like, all of which can be coated interiorly with the PNAs and other optional agents.

### EXAMPLES

The following non-limiting Examples demonstrate the efficacy of the PNAs in various applications. Supplementary methodology for each figure referred to can be found in the Brief Description of the Drawings.

### Example 1

Following their design by the inventors, the PNAs were manufactured and tested for purity by PNA Bio (Newberry Park, CA). Each sequence was linked to a cell penetrating peptide (CPP) with amino acid sequence (KFF) 3K. PNAs and CPPs were bound together with an O-linker (also known as egl or AEEA linker), a 9-atom organic molecule that serves as a spacer and improves solubility. They were shipped as a dry powder for reconstitution in the laboratory with sterile deionized water.

Clinical bacterial isolates were sub-cultured in Luria Broth (LB) medium and incubated dynamically overnight at 37° C., normalized to OD600=0.01, and 10-fold diluted in 2× minimal salt medium specific to the organism. A total of 60 μL (30 μL of the 10⁻³ dilution of each strain and 10 μM of each antisense-PNA in 30 μL in nucleases-free sterile water) was plated in a polystyrene 96-well plate.

After 24-48 hrs of static growth at 30° C., the contents of each well was plated on tryptic soya agar (TSA) to assess cell viability.

Biofilm formation was evaluated after wells were stained with 0.1% crystal violet, solubilized with 33% glacial acetic acid, and measured at OD_{590 nm}.

Cell viability was measured by stamping the liquid contents of each well onto TSA agar, then extracting 40 μL from each well and adding to 360 μL LB media and performing 10-fold dilutions up to 10⁻⁶.

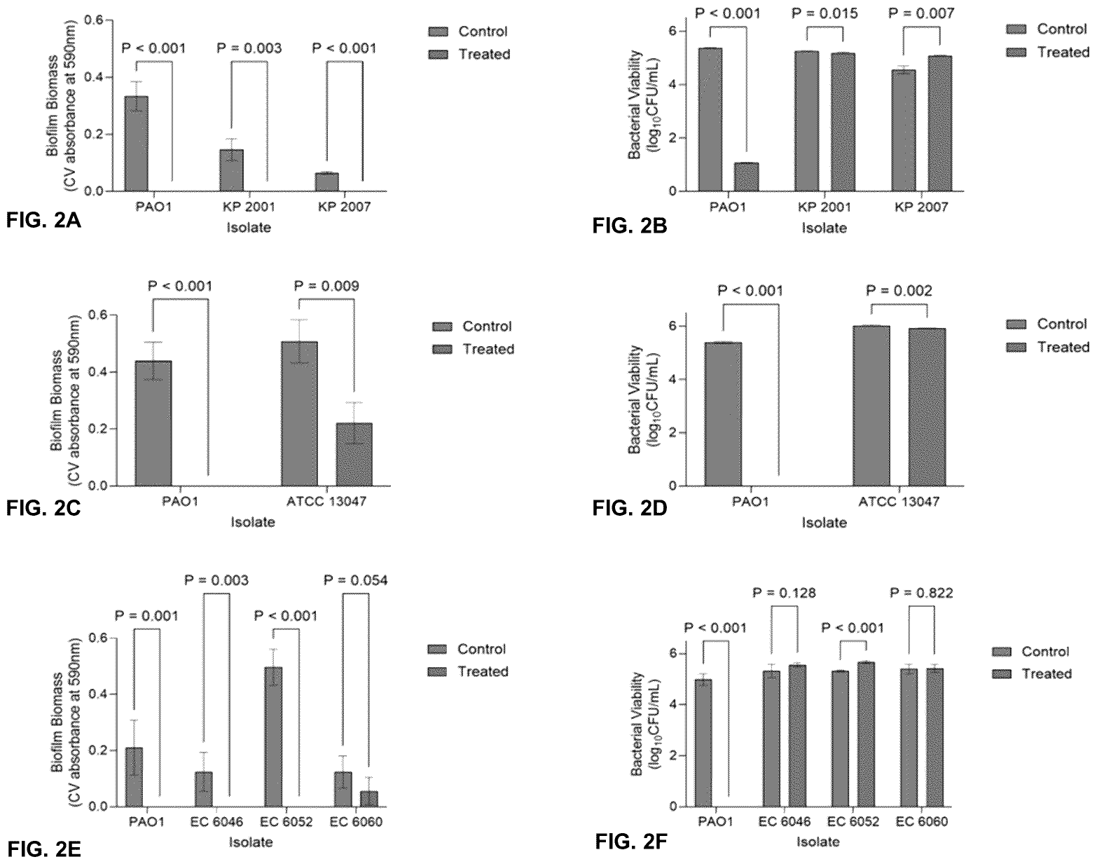

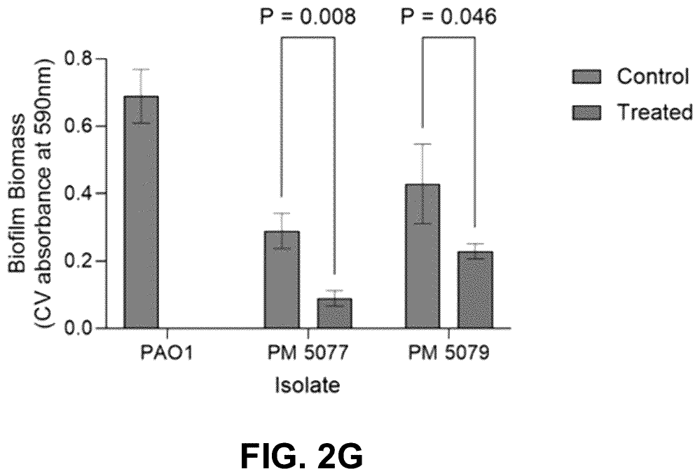

After 24 hrs of growth, PNAs inhibited biofilm formation, as shown in FIGS. 2A, 2C, 2E, 2G, and significantly reduced cell viability, as shown in FIGS. 2B, 2D, 2F.

In addition, wrPNAs were tested against individual intended targets (K. pneumoniae, E. cloacae, E. coli, and P. mirabilis); biofilm formation was assessed as described above. wrPNAs demonstrated reduction in biofilm against their intended individual targets, with variable impact on bacterial viability (FIGS. 2A-2G).

More detailed information about these experiments is provided in Example 4.

### Example 2

Polystyrene 96-well plates were coated with gentamicin (2 mg/mL) and different concentrations of polydopamine and biofilm and bacterial viability of gentamicin-sensitive biofilm forming laboratory strain P. aeruginosa (PAO1) and gentamicin resistant Pa383-ArahU::GM were assessed. Gentamicin combined with a low concentration PDA coating (0.25 and 0.5 mg/mL) inhibited P. aeruginosa (PAO1) biofilm formation and bacterial viability after 24 hrs incubation, as shown in FIGS. 3C and 3D.

### Example 3

Polystyrene 96-well plates coated were coated with motA 0 at increasing concentrations (10, 20 and 30 μM) and biofilm measured after 24 hrs incubation P. aeruginosa (PAO1). The motA 0 PNA combined with 0.25 mg/ml PDA inhibited PAO1 biofilm formation after 24 hrs incubation, as shown in FIG. 4.

### Example 4

### Abstract

Biofilms develop in sequential steps resulting in the formation of three-dimensional communities of microorganisms that are encased in self-produced extracellular polymeric substances. Biofilms play a key role in device-associated infections, such as catheter-associated urinary tract infections (CAUTIs), because they protect microorganisms from standard antimicrobial therapies. Current strategies to prevent biofilm formation in catheter-related infections, including prophylactic antibiotics and antibiotic-coated catheters, have been unsuccessful, highlighting a need for novel technologies. In this example, biofilm-forming phenotypes of common Gram-negative bacteria associated with CAUTIs were treated with antisense-peptide nucleic acids (PNAs) and biofilm biomass and bacterial viability were quantified after 24 or 48 hours of treatment. A cocktail of PNAs targeting the global regulator genes rsmA, amrZ, and rpoS in Pseudomonas aeruginosa significantly reduced biofilm biomass and viability. Antisense-wide-range PNAs against these same gene targets and the motility regulator gene motA inhibited biofilm formation among isolates of Klebsiella pneumoniae, Enterobacter cloacae, Escherichia coli, and Proteus mirabilis, but did not reduce bacterial viability. These results suggest that antisense-PNAs are a promising new technology in preventing biofilm formation in urinary catheters, especially as a potential complement to conventional antimicrobials.

### Introduction

Bacterial biofilms in the healthcare setting are critical to address because they pose a risk to patient health. It is estimated that more than 65% of nosocomial infections are due to biofilms, which are implicated in surgical site infections, bloodstream infections, and catheter-associated urinary tract infections (CAUTIs) (Assefa, M. & Amare, A, 2022). Up to 25% of hospitalized patients receive a urinary catheter, and roughly 75% of urinary tract infections developed in the hospital are associated with catheter use (U.S. Centers for Disease Control and Prevention, 2024). Biofilms form on medical devices and can confer resistance to antimicrobials and host immune defenses (Muhammad, M. H. et al., 2020) This resistance may arise by several proposed mechanisms, including conventional resistance mechanisms, the protective barrier formed by biofilms, and the multicellular nature of biofilms, which involves inter-bacterial signaling via quorum sensing (QS) (Muhammad, M. H. et al., 2020; Sharma, D., Misba, L. & Khan, A. U., 2019).

Gram-negative bacilli are the leading cause of CAUTIs, including Pseudomonas aeruginosa; Escherichia coli; and select Klebsiella spp., Enterobacter spp., and Proteus spp. (Weiner-Lastinger, L. M. et al., 2020; Stickler, D. J., 2014). Clinically, best practices related to urinary catheters include minimizing the frequency and duration of their use, and studies suggest antimicrobial prophylaxis in asymptomatic patients is not beneficial (Assadi, F., 2018). Attempts to address biofilms in the pathogenesis of CAUTIs include the use of antifouling and antibacterial surfaces leveraging polyethylene glycol or metal ions, silver alloy coated catheters, interrupting QS via degradation of signal molecules and quorum sensing inhibitors, and nanoparticle delivery of antibiotics (Muhammad, M. H. et al., 2020; Sharma, D., Misba, L. & Khan, A. U., 2019; Lila, A. S. A. et al., 2023; and Pickard, R. et al., 2012). These technological approaches have been insufficient in clinical use and challenged by multiantibiotic resistance, thus highlighting the need for novel methods to prevent bacterial biofilm formation in urinary catheters (Dubern, J.-F. et al., 2023)

Synthetic antisense-peptide nucleic acids (PNAs) can be harnessed to inhibit gene expression, and their use in prokaryotic systems was demonstrated in a biofilm-forming laboratory strain of P. aeruginosa (Hu, J., Xia, Y., Xiong, Y., Li, X. & Su, X, 2011; Narenji, H. et al., 2017; and Pellestor, F. & Paulasova, P., 2004). PNAs are synthetic DNA analogs created by replacing the negatively charged phosphate backbone of DNA with a neutral pseudopeptide backbone, with nitrogenous bases attached by methylene carbonyl bonds (Pellestor, F. & Paulasova, P., 2004; PNA Bio. Custom PNA Oligos, 2022; and Swenson, C. S. & Heemstra, J. M., 2012). PNAs form DNA and RNA hybrids, abiding by Watson-Crick-Franklin hybridization rules, with high specificity and selectivity. The resultant PNA-DNA and PNA-RNA complexes are more stable than the natural complexes since there is no electrostatic repulsion (Pellestor, F. & Paulasova, P., 2004; Swenson, C. S. & Heemstra, J. M., 2012; Menchise, V. et al., 2003). Additional properties of PNAs include high thermal stability, hybridization at almost any salt concentration, and resistance to proteases and nucleases (Pellestor, F. & Paulasova, P., 2004; and Menchise, V. et al., 2003). The desired PNA sequence is addended to a cell-wall permeabilizing peptide to improve entry of the PNA through cell membranes, and an O-linker, to improve solubility (Hu, J., Xia, Y., Xiong, Y., Li, X. & Su, X., 2011; and Rajasekaran, P. et al., 2013). While PNAs bind to targets with high specificity, this characteristic limits their effectiveness against a range of bacteria or polymicrobial infections.

In this example, novel antisense-PNAs were designed to inhibit biofilm formation, via targeting the reversible and irreversible attachment phases of planktonic bacteria to a surface3. The current work aims to prevent biofilm establishment rather than attempting to dislodge attached and mature biofilm. Additionally, antisense-wide-range PNAs (wrPNAs) were designed to offer a precise, yet potentially broad, biofilm inhibition technology, leveraging the fact that many of the key regulatory genes of biofilm formation are relatively conserved across Gram-negative bacilli implicated in CAUTIs.

### Results

### PNA Design

The PNAs in this example were specifically designed to target global regulator genes critical to the formation and maintenance of biofilms. The PNAs described are outlined in FIGS. 1A and 1B. The selected gene targets included rsmA (a translation regulator including of proteins involved in QS), amrZ (a transcription factor that regulates twitching motility and alginate synthesis), and rpoS (a stress response and biofilm architecture regulator) (Jones, C. J., Ryder, C. R., Mann, E. E. & Wozniak, D. J., 2013; Mika, F. & Hengge, R., 2014; Vakulskas, C. A., Potts, A. H., Babitzke, P., Ahmer, B. M. M. & Romeo, T., 2015). The flagellar motility gene motA is also recognized as a critical mediator of early biofilm establishment through its role in polar reversible attachment between bacteria and a surface as well as additional adherence events during the irreversible phase of biofilm development (Muhammad, M. H. et al., 2020; and Hu, J., Xia, Y., Xiong, Y., Li, X. & Su, X., 2011). Each PNA is labeled according to its intended gene target. The original PNAs were designed based on the PAO1 genome, and are designated as “0”. Subsequent wrPNAs with homologous targets are labeled sequentially (FIG. 1A).

A cocktail of PNAs inhibited biofilm formation in PAO1 and reduced bacterial viability

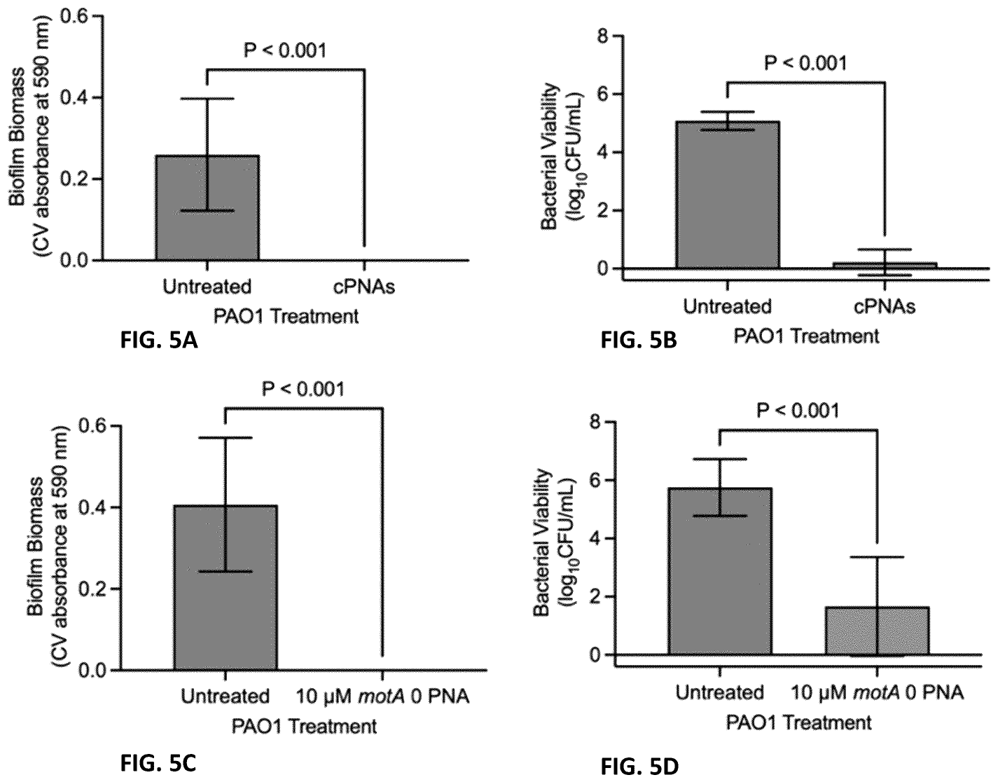

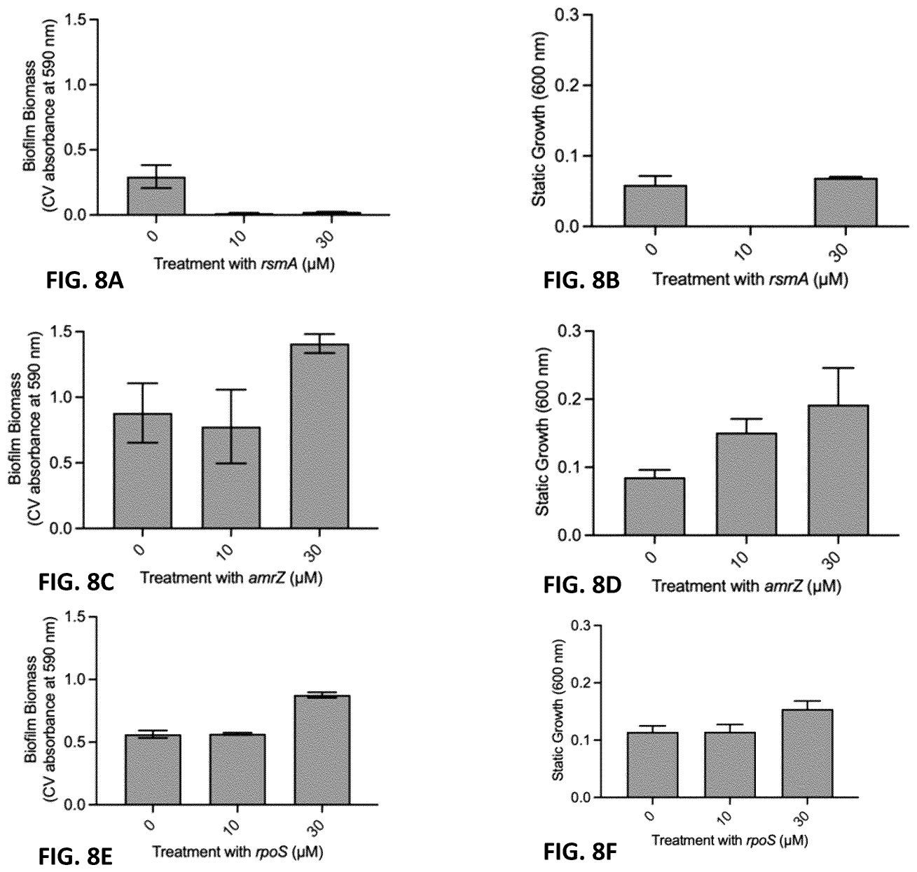

Prior studies demonstrated inconsistent effects of single antisense-PNA treatment on PAO1 biofilm formation and bacterial viability (FIGS. 8A-8F). Therefore, combination treatment with multiple PNA targets was pursued. 24 h treatment with an equimolar cocktail of the three antisense-PNAs rsmA 0, amrZ 0, and rpoS 0 (cPNAs) eliminated biofilm formation in PAO1 (P<0.001) and significantly reduced average bacterial viability by a fold change of 0.043 (P<0.001) (FIGS. 5A and 5B and FIG. 1A). 24 h treatment with motA 0 antisense-PNA eliminated biofilm formation (P<0.001) and significantly reduced average bacterial viability by a fold change of 0.289 (P<0.001) (FIGS. 5C and 5B and FIG. 1A).

Antisense-wrPNAs inhibited biofilm formation in their intended species with no meaningful reduction in bacterial viability

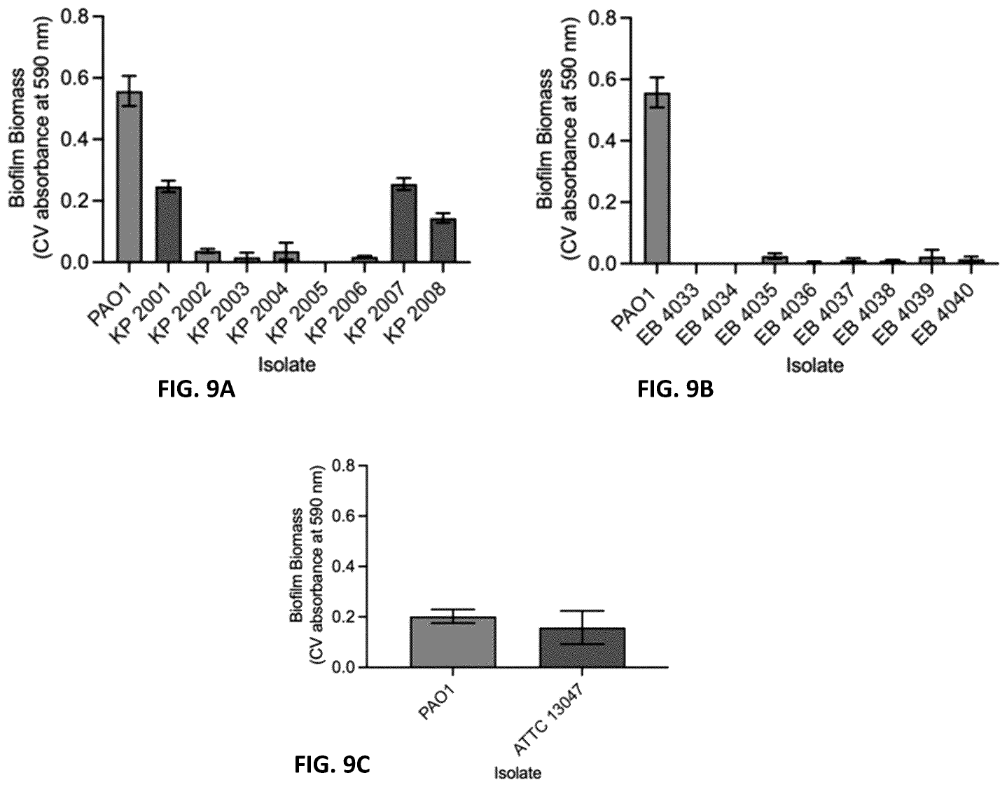

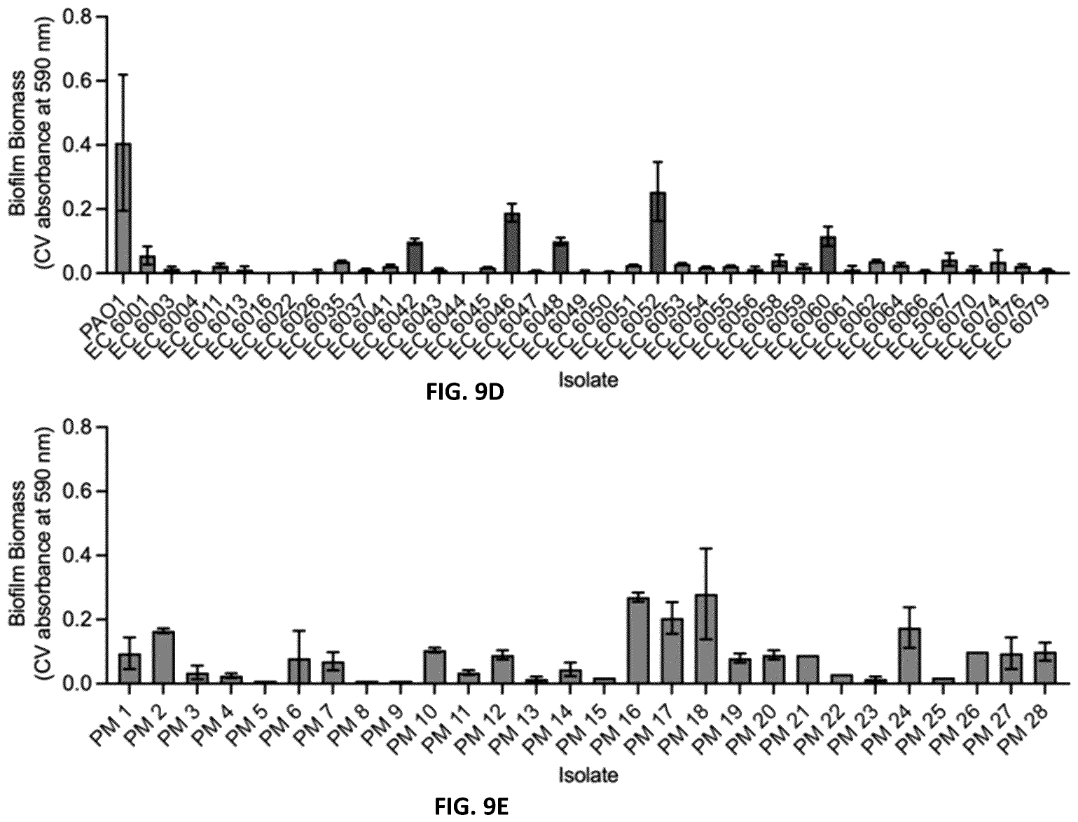

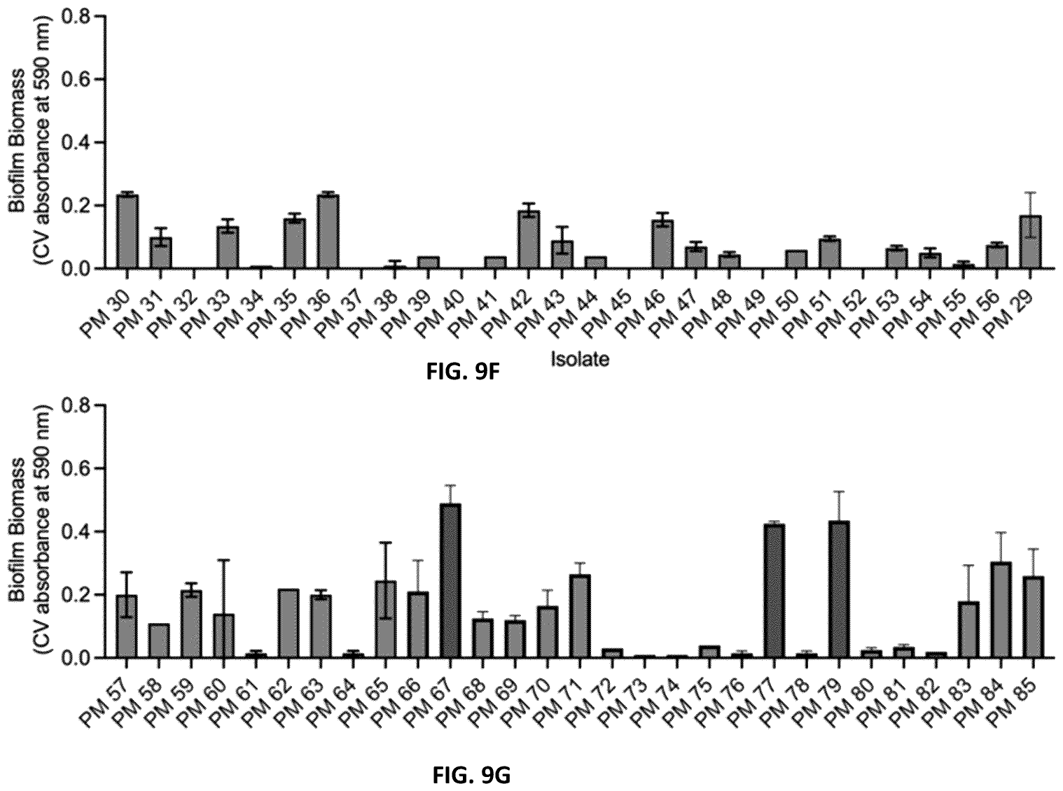

Biofilm-formation by clinical isolates of K. pneumoniae, E. cloacae, E. coli, and P. mirabilis was quantified. A biofilm-forming phenotype was qualified as an average biofilm biomass greater than or equal to 0.1. Biofilm-forming clinical isolates were identified for all species investigated except E. cloacae (EB), so a commercially purchased strain of E. cloacae (ATTC 13047) was utilized (FIG. 9B).

Antisense-wrPNA targets were designed for bacterial species (FIGS. 1A and 1B). 24 h antisense-wrPNA treatment with rsmA I eliminated biofilm biomass in K. pneumoniae (P<0.005) with no meaningful reduction in bacterial viability (FIGS. 2A and 2B). 24 h antisense-wrPNA treatment with an equimolar cocktail of rsmA 1 and amrZ 2 significantly reduced average biofilm biomass in commercial E. cloacae strain ATCC 13047 by a fold change of 0.436 (P<0.01), and there was no meaningful reduction in bacterial viability (FIGS. 2C and 2D). 24 h antisense-wrPNA treatment with an equimolar cocktail of rsmA 1 and amrZ 1 eliminated biofilm biomass in two of the three clinical isolates of E. coli (P<0.005), but insignificantly reduced average biofilm biomass in isolate EC 6060 (P=0.054) (FIG. 2E). There was no meaningful reduction in average bacterial viability with 24 h antisense-wrPNA treatment of E. coli (FIG. 2F). After 48 h antisense-wrPNA treatment with an equimolar cocktail of rsmA 2, rpoS 1, and motA 1, average biofilm biomass was significantly reduced in P. mirabilis by a log change of 0.311 for clinical isolate PM 5077 (P<0.01) and 0.536 for clinical isolate PM 5079 (P<0.05) (FIG. 2G). Bacterial viability upon antisense-wrPNA treatment of P. mirabilis was not assessed in this example.

Antisense-PNAs reduced biofilm biomass in polymicrobial samples

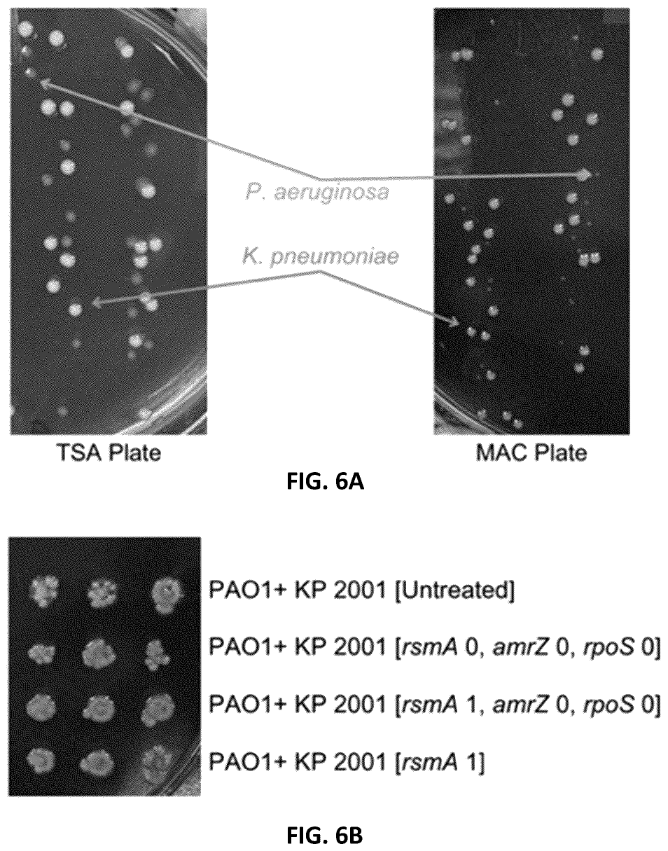

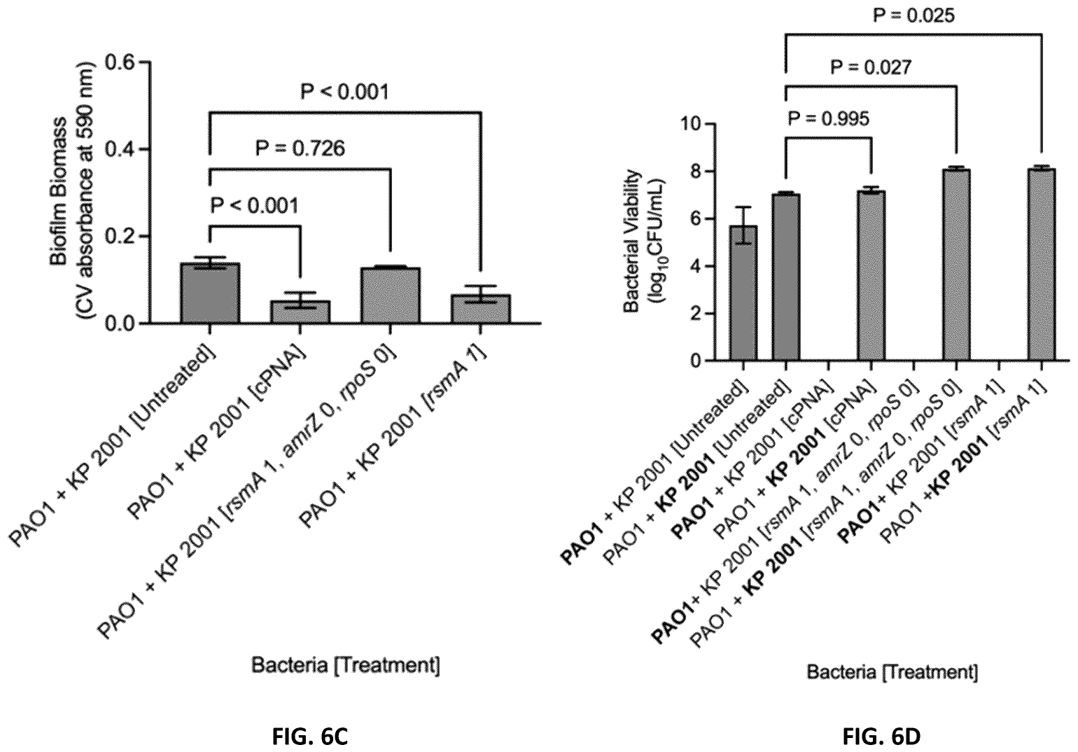

K. pneumoniae, a lactose-fermenter, was cultured at a 1:1 ratio with PAO1, a non-lactose fermenter, and this was confirmed morphologically (FIG. 6A). After 24 h incubation, qualitative assessment of bacterial viability suggested dominance of K. pneumoniae with antisense-PNA treatment as evident by the lack of non-mucoid PAO1 (FIG. 6B). Upon cPNAs treatment, average biofilm biomass was significantly reduced by a log change of 0.382 (P<0.001), PAO1 bacterial viability was eliminated, and K. pneumoniae bacterial viability was unchanged compared to untreated control (FIGS. 6C and 6D). Upon switching the rsmA 0 antisense-PNA in the cPNAs with rsmA 1 antisense-wrPNA, the reduction in biofilm biomass was lost (P=0.726); however, PAO1 viability was still eliminated (FIGS. 6C and 6D). When the polymicrobial sample was treated with antisense-wrPNA rsmA 1 only, there was still a significant reduction in average biofilm biomass compared to untreated control with a log change of 0.482 (P<0.001), and PAO1 bacterial viability was still eliminated (FIGS. 6C and 6D). Regardless of whether the polymicrobial sample was treated with an equimolar cocktail of rsmA 1, amrZ 0, and rpoS 0 or rsmA 1 only, there was no favorable reduction in K. pneumoniae bacterial viability (FIG. 6D).

### Dried Antisense-PNAs Retained Anti-Biofilm Effect

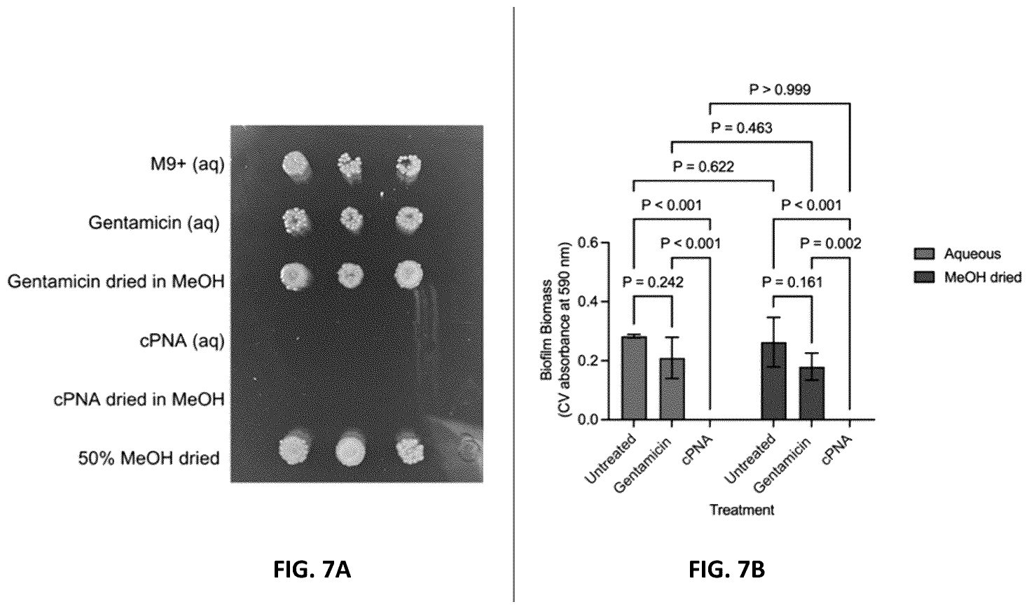

24 h incubation with cPNAs eliminated bacterial viability of gentamicin-resistant P. aeruginosa, Pa383-ArahU::GM, regardless of whether the cPNAs were in solution (aq) or dried onto a 96-well plate with methanol (MeOH) (FIG. 7A). Average biofilm biomass after 32 h incubation was not significantly different between untreated Pa383-ArahU::GM in standard M9+ media compared to in 50% MeOH dried control wells (P=0.622) (FIG. 7B). There was no statistically significant difference between the average biofilm biomass of Pa383-ArahU::GM treated with gentamicin (aq) versus gentamicin dried in MeOH (P=0.463), untreated bacteria in M9+ media verses gentamicin (aq) (P=0.242), or untreated bacteria in dried MeOH control wells compared to gentamicin dried in MeOH (P=0.161) (FIG. 7B). 32 h incubation with cPNAs eliminated biofilm biomass in the aq and dried forms (FIG. 7B).

### Discussion

CAUTIs adversely impact patient health and the financial wellbeing of healthcare systems. Bacteria within biofilms are 10 to 1,000 times more resistant to antibiotics than planktonic bacteria, which confers serious implications in the management of microbial infections secondary to bacterial biofilms (Sharma, D., Misba, L. & Khan, A. U., 2019). Additionally, in the United States alone, the total economic burden of CAUTIs was estimated to be $1.7 billion annually (in 2016 dollars), highlighting both clinical and economic motivations to reduce the incidence of CAUTIs (Hollenbeak, C. & Schilling, A., 2018).

In this example, we demonstrated that antisense-PNAs are a promising solution to prevent biofilm establishment among many Gram-negative bacteria associated with CAUTIs. Inspired by the combination therapies used in a variety of conditions, such as human immunodeficiency virus, we investigated treating PAO1 with a cocktail of equimolar rsmA 0, amrZ 0, and rpoS 0 (cPNAs), which eliminated biofilm formation and bacterial viability suggesting a beneficial effect of simultaneously targeting multiple genes involved in biofilm formation and maintenance (FIGS. 5A and 5B) (Kemnic, T. R. & Gulick, P. G., 2022). Hu et al. previously demonstrated the functionality of a cell-penetrating peptide, and that antisense motA PNA treatment reduced biofilm formation in PAO1 in a dose-dependent manner (Hu, J., Xia, Y., Xiong, Y., Li, X. & Su, X., 2011). However, among the probe sequences Hu et al. assessed, the impact of antisense-PNA treatment on bacterial viability was not reported; although, decreased motility upon antisense-PNA treatment was observed (Hu, J., Xia, Y., Xiong, Y., Li, X. & Su, X., 2011). In the present example, we demonstrated elimination of biofilm formation and a significant reduction in average bacterial viability upon antisense-PNA motA 0 treatment (FIGS. 5C and 5D). This difference in effect highlights the importance of PNA sequence design (FIG. 1A).

To the best of our knowledge, this is the first time antisense-PNAs have been designed to intentionally encompass sequences of genes that are conserved among multiple bacterial species (referred to here as antisense-wrPNAs). Further, we assessed the impact of those antisense-wrPNAs on biofilm biomass and bacterial viability. This broadening of PNA specificity is best exemplified by the antisense-wrPNA rsmA 1, whose sequence is applicable to Enterobacter spp., Klebsiella spp., E. coli, and P. vulgaris (FIG. 1A). Given historical findings that individual antisense-PNA treatment in PAO1 resulted in inconsistent effects, it was not entirely surprising that antisense-wrPNA treatment did not reduce bacterial viability in the intended species (FIGS. 2A-2G). Future work will be directed at investigating combination therapy that includes both antisense-wrPNAs and conventional antibiotics to generate a bactericidal effect. However, it was reassuring that biofilm biomass was reduced with antisense-wrPNA treatment, as the gene targets are global regulators of biofilm formation and maintenance, not necessarily viability (FIGS. 2A-2G). Additional studies could examine differences at the genomic and proteomic levels regarding the discordance between the anti-biofilm effect among the E. coli clinical isolates (FIG. 2E). Identification of novel, shared genes associated with prominent biofilm-forming phenotypes could offer new targets for antisense-wrPNA design.

Given that polymicrobial colonization is common among catheterized patients, ranging from 31-87% depending on the study, polymicrobial samples were investigated in the laboratory (Nye, T. M. et al., 2024). Tryptic soy agar and MacConkey agar were sufficient for differentiating the Gram-negative bacilli in this example based on phenotypic differences (FIGS. 6A and 6B). The loss of anti-biofilm effect of antisense-wrPNA rsmA 1 when combined in an equimolar cocktail with antisense-PNAs amrZ 0 and rpoS 0 was unexpected (FIG. 6C). Binding among the antisense-(wr) PNAs likely did not occur, as PAO1 viability was still eliminated with rsmA 1, amrZ 0, and rpoS 0 cocktail (FIG. 6D). Instead, downstream effects from the cPNAs may be responsible, which requires further investigation at the genomic and proteomic levels. The consistent reduction in PAO1 bacterial viability upon treatment suggests that the two base pair difference in the sequence was not sufficient to prevent an effect from the antisense-wrPNA designed for Enterobacter spp., Klebsiella spp., E. coli, and P. vulgaris on PAO1 (FIG. 6D and FIG. 1A). PNA literature describes the peptide-like backbone of antisense-PNAs as enhancing specificity, though the effect that antisense-wrPNAs have on specificity requires additional study (Swenson, C. S. & Heemstra, J. M., 2012). Future work could be directed at characterizing the individual contribution of different bacterial species to overall biofilm biomass, and assessing other combinations, or more complex combinations, of polymicrobial samples. Additionally, future studies could evaluate the extent of PNA integration by quantifying the target gene expression.

Ultimately, antisense-wrPNA technology holds potential for a range of applications, including as a coating on the luminal surface of catheters to prevent biofilm formation and bacterial viability. Drying the cPNAs onto the surface of 96-well plates was a preliminary trial in increasing clinical applicability, and consistent biofilm formation between the untreated conditions supported use of this methodology (FIGS. 7A and 7B). Further, the aqueous and dried cPNAs conditions both completely eliminated biofilm biomass and bacterial viability, suggesting that the return to a lyophilized state did not inherently eliminate the ability of the cPNAs to penetrate bacteria, hybridize to bacterial DNA, or exert their intended bioactivity (FIGS. 7A and 7B).

Antisense-wrPNAs could be used to prevent biofilm-associated infections in other contexts, including surgical meshes, burn wounds, pancreatic and biliary stents, and implantable medical devices (Su, Y. et al., 2022). This approach is particularly relevant for patients with long-term urinary catheters, such as for urinary retention, bladder outlet obstruction, and pelvic fracture. For catheters colonized with polymicrobial bacteria, antisense-wrPNAs capitalize on genetic conservation among commonly implicated bacterial species in CAUTIs. In addition, antimicrobials could be used in combination with effective antisense-wrPNAs in settings of chronic catheterization. Thus, antisense peptide nucleic acids offer a promising solution to many clinically-relevant problems.

### Methods

### Bacterial Strains

Biofilm-forming laboratory strain of P. aeruginosa PAO1; biofilm-forming clinical isolates of K. pneumoniae, E. coli, P. mirabilis; and biofilm-forming laboratory strain of E. cloacae (ATCC 13047) were utilized (Supplementary methods for screening methodology). 25-30% (vol/vol) glycerol stocks of bacteria were streaked onto Tryptic Soy Agar (TSA) or MacConkey Agar (MAC) plates and incubated overnight at 37° C. Single colonies were then subcultured in 4 mL of Luria Bertani (LB) Miller's media overnight at 37° C. in shaking conditions (200 rpm). This study was evaluated by the Carilion Institutional Review Board (IRB-23-1844) because of the use of clinical isolates; it received a non-human subjects research determination.

### Antisense-PNA Design and Synthesis

Antisense-PNAs were designed using NCBI Blast to select a 12 to 14 base pair region inclusive of the start codon. The start codon was included because the PNAs were designed to prevent transcription of the target gene (FIGS. 1A and 1B). A PNA Tool (PNA Bio, Newberry Park, CA) was used to confirm optimal design. The 12 to 14 base pair range was determined from previous literature reports that suggested at least a 10-mer PNA is needed to form stable PNA-DNA hybrids, while at the same time the PNA needs to be long enough to bind to a targeted area of a specific gene (Pellestor, F. & Paulasova, P., 2004). In wide-range PNAs, the given base pair sequence is found in the gene of multiple bacterial species; therefore each is assigned a number (i.e: 0, 1, 2) for identification (FIGS. 1A and 1B). Each sequence is conjugated to a cell-penetrating peptide, (KFF)3K, to improve cell wall-permeability and a 9-atom organic compound (O-linker) to improve solubility (Hu, J., Xia, Y., Xiong, Y., Li, X. & Su, X., 2011). All antisense-PNAs were synthesized and assessed for purity by PNA Bio (Newberry Park, CA), and then shipped as a dry powder for reconstitution in the laboratory. The sequences and manufacturing details are delineated in our provisional patent 63/643,580. Nammalwar Sriranganathan, Ph.D. procured the antisense-(wr)PNAs.

### Antisense-PNA Treatment

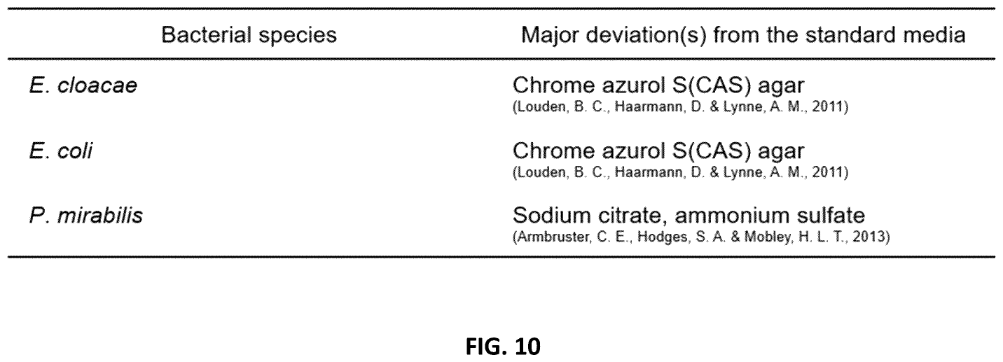

Bacterial subcultures were normalized to optical density (OD) OD 600=0.01 in minimal M9+ media (supplemented with sterile 20% glucose, 1 M MgSO₄, 0.1 mM CaCl₂, 3% casamino acid in 8-hydroxyquinolone solution). Standard diluted tubes were made utilizing bacteria specific media (FIG. 10). The standardized subcultures contained roughly 1.7×107 CFU/mL, which was verified by serial dilution and quantification of CFU/mL in triplicates. For a given 96-well clear flat bottom polystyrene tissue culture-treated microplate, the final volume was 60 μL and consisted of the following unless otherwise stated: 10 μM per antisense-(wr) PNA, 30 μL of 1.7×107 CFU/mL bacterial solution, and the remaining volume was sterile deionized water. Each condition was performed in triplicate. The plate was incubated statically at 30° C. for 24 or 48 h in a humid chamber. Biofilm-forming laboratory strain of P. aeruginosa, PAO1, was used as a positive control. 10 μM per antisense-(wr) PNA was based on the literature exploring concentration of PNA required to inhibit bacterial growth (Rajasekaran, P. et al., 2013).

For polymicrobial biofilm, the 30 μL of normalized bacteria described above consisted of 30 μL total of equal parts of 1.7×107 CFU/mL PAO1 and 1.7×107 CFU/mL of a second bacterial species.

30 μL of standard dilutions were streaked and counted in duplicate on TSA or MAC plates to ensure consistent bacterial input, which was measured in CFU.

### Drying cPNAs onto 96-Well Plates

An equimolar (10 μM per antisense-PNA) cocktail of the antisense-PNAs rsmA 0, amrZ 0, and rpoS 0 with 50% methanol (MeOH) (total volume: 60 μL) was dried onto a 96-well clear flat bottom polystyrene tissue culture-treated microplate in a Fisherbrand™ Isotemp™ Digital Dry Bath/Block Heater at 37° C. 60 μL of 100 μg/mL gentamicin was also dried onto wells. Gentamicin-resistant P. aeruginosa, Pa383-ΔrahU::GM was subcultured as described above (Rao, J. et al., 2011)

### Bacterial Viability Assessments

Direct stamping on TSA or MAC plates was performed first after antisense-PNA treatment was complete. Then, 10-fold serial dilutions of each well or tube were performed in LB media. 30 μL of each dilution, in duplicate, was streaked on TSA or MAC plates to determine CFU. The plates were incubated overnight at 37° C., and each plate was read by ≥2 readers.

For polymicrobial samples, the serial dilutions were plated on TSA and MAC plates to highlight phenotypic differences.

### Biofilm Biomass Quantification

Biofilms were stained with 175 μL 0.1% crystal violet (CV), rinsed with sterile water until the water ran clear, solubilized in 100 μL of 33% glacial acetic acid (GAA), and absorbance was measured using a spectrophotometer at 590 nm (Wilson, C. et al., 2017).

### Statistical Analysis

Results are presented as mean±standard deviation (s.d.). P values were determined using unpaired t-tests (α=0.05), one-way analysis of variance (ANOVA) with follow up tests comparing the mean of each column to the control column (α=0.05), or two-way ANOVA with multiple comparisons comparing cell means with others in its row and its column (family-wise a threshold and confidence level=0.05). All graphs and statistics were created or performed in GraphPad Prism 10.

### Supplementary Methods

### Screening for Biofilm-Formation Among Clinical Isolates

A Gram-negative repository of over 700 clinical isolates from blood, urine, and respiratory specimen collected from Carilion Clinic patients (Roanoke, VA) was created by Jayasimha Rao, Ph.D. and Anthony W. Baffoe-Bonnie, MD from 2009 to the present (IRB No. 1263). 25-30% (vol/vol) glycerol stocks of clinical isolates of K. pneumoniae, E. cloacae, E. coli, and P. mirabilis (stored at −80° C.) were streaked onto Tryptic Soy Agar plates and incubated overnight at 37° C. Single colonies were subcultured in 4 mL of Luria Bertani (LB) Miller's media overnight at 37° C. in shaking conditions (200 rpm). Bacterial subcultures were normalized to optical density (OD) 600=0.01 in LB media. 60 μL of a standard diluted 10-3 tube was plated in duplicate or triplicate in a 96-well clear flat bottom polystyrene tissue culture-treated microplate, incubated statically at 30° C. for 24 or 48 h in a humid chamber, and biofilm biomass was quantified as described in the main methods section under “Biofilm biomass quantification”. Biofilm-forming laboratory strain of P. aeruginosa, PAO1, was used as a positive control.

### Example 5

### Abstract

Urinary catheterization is a common procedure, affecting 15-25% of hospitalized patients. However, this procedure often results in bacterial infections, primarily caused by biofilm-forming Gram-negative bacteria that adhere to the catheter surface. To address this challenge, we developed a polymer-based antimicrobial coating using polydopamine (PD) embedded with gentamicin (PD-Gent). To evaluate the antibiofilm efficacy of this coating, we used a biofilm-forming laboratory strain of Pseudomonas aeruginosa (PAO1). Pseudomonas spp., are also a common pathogen implicated in catheter-related infections, that was incubated either in the presence or absence of the PD-Gent composite. This coating successfully reduced biofilm formed by PAO1. The versatility of this approach also enables other antibiotics to be incorporated into the coating material (e.g., tobramycin), and a variety of surfaces to be coated, including poly (vinyl chloride), polystyrene, and silicone. The PD-Gent coating also showed high antibiofilm activity compared to the uncoated control under in vitro conditions using artificial urine and constant bacterial flow overnight.

### Introduction

Bacterial biofilms contribute to the spread of infectious disease and are implicated in the persistence of chronic infection (Preda, V. G.; Sandulescu, O., 2019; Mendhe, S.; Badge, A.; Ugemuge, S.; Chandi, D., 2023) Biofilm formation first involves free-roaming (planktonic) bacteria attaching to a surface. Once attachment is achieved, a microcolony is formed, which produces extracellular polymeric substances (EPS) to which the bacteria can adhere. Following biofilm formation planktonic cells can be liberated from the EPS matrix and dispersed to form new biofilms. Due to the EPS, diffusion of antibiotics is slowed, making biofilms inherently recalcitrant to traditional antibiotics and invading by immune system, which are critical in the pathogenesis of device-associated infections (Vertes, A.; Hitchins, V.; Phillips, K. S., 2012; Zafer, M. M., Mohamed, G. A., Ibrahim, S. R. M., Ghosh, S., 2024; and Lebeaux, D.; Ghigo, J. M.; Beloin, C., 2014). Consequently, bacterial biofilms significantly impact patients with indwelling hardware, including urinary catheters, central lines, and artificial prostheses. Several strategies are employed to prevent and/or mitigate biofilm-related, device-associated infections, including early device removal, systemic antibiotics, and antimicrobial lock therapy. However, these approaches are often ineffective and, in some circumstances, facilitate the emergence of antimicrobial resistance.

Catheter-associated urinary tract infections (CAUTIs) are a clinically significant biofilm-related disease, representing the leading cause of hospital-related infections, accounting for over 75% of urinary tract infections (Al-Qahtani, M., Safan, A., Jassim, G., Abadla, S., 2019; and Nicolle, L. E., 2014). In these cases, CAUTIs are often treated with systematic antibiotics and catheter replacement; however, systemic antibiotic use is not recommended for prophylactic purposes (Flores-Mireles, A., Hreha, T. N., Hunstad, D. A., 2019). To mitigate CAUTIs, various antimicrobial catheters have been developed (Vazquez-Rodriguez, J. A. et al., 2022; Hemmatpour, H. et al., 2023; and Goda, R. M., 2022). Unfortunately, no antimicrobial catheter has demonstrated clear and consistent biofilm inhibition and consistently effective prevention of CAUTIs remains an unmet clinical need (Gambrill, B. et al., 2024); Thus, new strategies for addressing CAUTIs and other biofilm-related infections are essential.

Catheter coatings must adhere to clinically realistic properties. For example, a coating must allow the catheter to retain its flexibility and tensile strength. Other considerations include maintaining a small enough outer diameter to ensure patients' comfort yet also having a wide enough inner lumen to prevent blockage. Polydopamine (PD)-based biomaterials have the ability to naturally adhere to surfaces and form thin uniform films as well as form of nanoparticles (PDNPs) that encapsulate small molecules for drug delivery (Battaglini, M., Emanet, M., Carmignani, A., Ciofani, G., 2024; Wang, B., Yuan, T., Zha, L., Liu, Y., Chen, W., Zhang, C., Bao, Y., Dong, Q., 2021; Chen, N., Yao, S., Li, M., Wang, Q., Sun, X., Feng, X., Chen, Y., 2023; Li, Y. et al., 2022; and Ren, D., Williams, G. R., Zhang, Y., Ren, R., Lou, J., Zhu, L.-M, 2022). However, PDNPs-based materials often suffer from poor scalability and dispersity control, limiting its applicability in healthcare. Antibiotics combined with silver nanoparticles (AgNPs) has also been explored, but AgNPs often suffer from particle aggregation and dose-dependent cytotoxicity (Batul, R., Bhave, M., Yu, A., 2023; Necula, B. S., et al., 2012; Fu, Y, et al, 2021;Ma. K, et al., 2020).

Inspired by established techniques using PD in the biomedical field, we designed a catheter coating where PD enhances the immobilization and proliferation of antimicrobial agents onto the catheter surface. This coating leverages the natural adhesion properties of PD without the synthetic limitations of nanoparticles (Yang, W., Liu, C., Chen, Y., 2018; and Murari, G., Bock, N., Zhou, H., Yang, L., Liew, T., Fox, K., Tran, P. A, 2020). Here, our coating composite is composed of gentamicin (an aminoglycoside antibiotic) and PD (Hemmatpour, H., 2023; and Lim, K, et al. 2015). Although widely used, aminoglycosides exhibit relatively low resistance rates compared to other antimicrobial agents, making them effective treatments against Pseudomonas aeruginosa (a common CAUTI-causing pathogen) (Rukholm, G., Mugabe C Fau-Azghani, A. O., Azghani Ao Fau-Omri, A., Omri, A, 2006, Harbarth, S.; Rohner, P., Safran, E., Garbino, J., Auckenthaler, R., Pittet, D., 1998, Cole, S. J., Records, A. R., Orr, M. W., Linden, S. B., Lee, V. T., 2014). The goal of this work was to create a versatile and easily adaptable approach that can be applied using various antimicrobial agents and to coat multiple types of surfaces all while preventing biofilm formation.

### Materials and Methods

### Materials

Sodium chloride, tobramycin, calcium chloride, gentamicin sulfate, dopamine hydrochloride, urea, and bovine serum albumin (BSA) were purchased from Sigma Aldrich. Disodium sulfate, tris base, and ammonium chloride were purchased from Oakwood. Hydrochloric acid, trisodium citrate dihydrate, and crystal violet were purchased from Fischer Scientific. Sodium oxalate was purchased from Thermo Fisher Scientific. Potassium phosphate monobasic was purchased from Acros. Tryptone soy broth was purchased from Oxoid. All chemicals were used as-is.

### Methods

### Liquid Chromatograph Mass Spectroscopy (LCMS)

An Agilent (Santa Clara, CA) 1260 HPLC system equipped with a Binary pump (1312B), autosampler (1329B), oven heater (1316B) and single quad MS as a detector was used for all separations and detections. A C8 column (250×4.6, 5 μm) from Agilent was used to obtain the separation of gentamicin. Analysis was performed using isocratic 98/2% (0.1% Formic acid/MeOH+0.1% FA). The flow rate was maintained at 0.8 mL/min. Most of the detections were obtained in scan (50-600 amu); however, for low concentration samples, detections were obtained in selected ion monitoring (SIM) mode using ion 478.4 amu.

### Poly(Vinyl Chloride) (PVC) Tube Sterilization

Tygon® PVC tubing was cut into 25 cm segments and placed in a 500 mL flask to sterilize before undergoing coating. To begin sterilization, 200 mL of 200 proof ethanol and 200 mL of deionized (DI) water were added to the flask, which was then sonicated for 30 minutes. After removing the solvent, the tubes were rinsed with DI water and dried in a 60° C. oven in preparation for polymerization.

### Synthesis of PD-Gent Coating

Tris-buffer was prepared by dissolving Tris-base (0.5 M) in DI water and adjusting the pH to 8.5. Dopamine hydrochloride was dissolved in 24 mL Tris-base before adding gentamicin sulfate (2 mg/mL). This solution was added to a 6-dram vial, equipped with a stir-bar and 25 cm of sterilized PVC tubing cut in 5 cm increments. The vial was heated to 60° C. with magnetic stirring and removed after 22 h. PVC tubing was subsequently rinsed with DI water until the solution ran clear, followed by oven drying at 60° C. An aliquot (3 mL) of reaction solution was collected and passed through a 0.45 μm syringe filter for liquid chromatography analysis. A similar procedure was used for well-plate studies using 0.150 mL of solution per well, followed by oven heating at 60° C.

### Bacterial Cell Culture

A single, PAO1 bacterial colony was sub-cultured in 4 mL of Luria Bertani (LB) medium overnight at 37° C. in shaking conditions (200 rpm). Bacterial subcultures were normalized to optical density OD₆₀₀=0.01 (solution standard) in minimal M9+ media (supplemented with sterile 20% glucose, 1 M MgSO4, 0.1 mM CaCl2, 3% casamino acid in 8-hydroxyquinolone solution). The solution standard was diluted three-fold before incubating in the PVC tubes or polystyrene (PS)-well plates. For the in vitro urinary model, bacterial subcultures were grown in LB medium and diluted using artificial urine to an OD₆₀₀=0.01 standard, which was further diluted four-fold before it was added to the feeding bag.

### Crystal Violet (CV) Staining

Biofilm biomass was determine using a previous method, which quantified biofilm density by staining with 0.1% CV solution (Adetunji, V., 2011; Grossman, A. B., Burgin, D. J., Rice, K. C, 2021; Wilson, C. et al., 2017; and Xu, Z., Liang, Y., Lin, S., Chen, D., Li, B., Li, L., Deng, Y., 2016). The liquid content of the wells and PVC tubes was removed and used to assess bacterial viability (described below). The staining procedure followed similar methods previously published (Kamimura, R. et al., 2022; Merritt, J. H., Kadouri, D. E., O'Toole, G. A, 2005; and Zegans, M. E., Wagner, J. C., Cady, K. C., Murphy, D. M., Hammond, J. H., O'Toole, G. A., 2009). Well plates and PVC tubes were stained with 100 and 60 μL (respectively) of a 0.1% crystal violet (CV) solution, then gently rinsed with 10 mL DI water until the rinsed water was clear. CV was solubilized in 100 μL of 33% glacial acetic acid and absorbance was measured at OD590 nm.

### Crystal Violet (CV) Background Staining

Non-specific binding between the PD, gentamicin, and CV solution was observed. To address this background interference, we incubated tubes and well-plates with M9+ solution without bacteria. These were subsequently stained with CV so that the background noise could be corrected for.

### Bactericidal Studies

Following incubation for 24 h, bacterial viability was assessed qualitatively by direct stamping onto Tryptic Soy Agar (TSA) plates. Additionally, the liquid content from the 96-well plates and PVC tubes was 10-fold serial diluted to quantify colony forming units, which were assessed by 2-3 observers.

### PAO1-Artificial Urine Solution for In Vitro Artificial Urinary Tract Model

Artificial urine, containing a mixture of protein and various salts, was made using previous methods in the literature (Sarigul, N., Korkmaz, F., Kurultak, İ. A, 2019; 36). Using the bacterial culture method described above, 40 mL of the three-fold PAO1 dilution was added to 360 mL of artificial urine, mixed, then subsequently poured into a sterile reservoir simulating a bladder.

### Results and Discussion

PD-based coatings have been widely studied for biomedical applications due to their low cost and toxicity (Davidsen, M. B., Teixeira, J. F. L., Dehli, J., Karlsson, C., Kraft, D., Souza, P. P. C., Foss, M, 2021). To modify surfaces using PD, an oxidative reaction occurs between dopamine and oxygen under alkaline conditions (Davidsen, M. B., Teixeira, J. F. L., Dehli, J., Karlsson, C., Kraft, D., Souza, P. P. C., Foss, M, 2021). The catechol and amine groups present in PD enable it to deposit and stick onto various types of surfaces (Davidsen, M. B., Teixeira, J. F. L., Dehli, J., Karlsson, C., Kraft, D., Souza, P. P. C., Foss, M, 2021) Combining PD with an antibiotic leverages the electrostatic interactions between catechol and amines in the polymer and gentamicin, respectively. To synthesize the PD-Gent coating, dopamine polymerizes into a thin film on the surface of catheter tubes in the presence of gentamicin. Here, poly(vinyl chloride) (PVC) tubing, a material also common in catheter tubing, is used to simulate the surface of catheters (Gambrill, B. et al., 2024; and Karmarkar, R., Bodapati, S., Yao, L., Aroori, S. A., 2024).

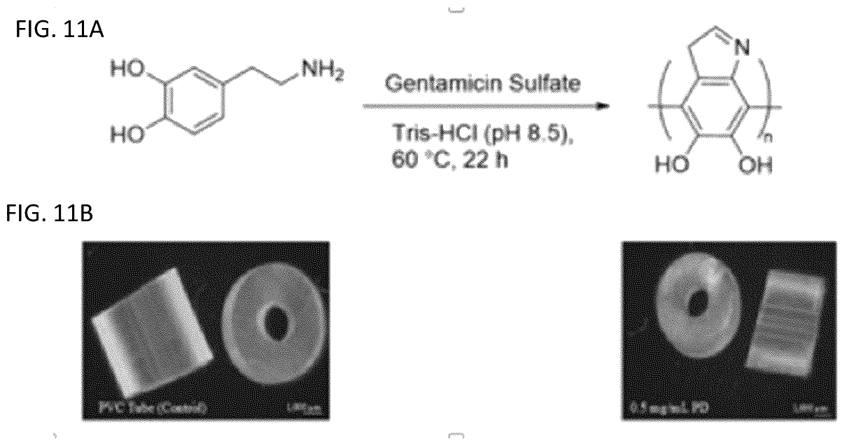

To evaluate the surface characteristics of the coating on the PVC tubes, scanning electron microscopy with energy dispersive x-ray spectroscopy (SEM-EDX) was used. However, neither the coating depth nor any heterogeneities could be determined. This limitation is likely due to the 1 mm penetration depth of the electron, exceeding the coating thickness. Optical microcopy, however, revealed a visible color change on the PVC surface, indicating coating deposition did not penetrate through the entire PVC tube. (FIG. 11B). Instead, we observed a clear dark layer in the PD-Gent coated tubes compared to the uncoated PVC (control) tubes, indicating PD deposition. We compared the coating images between trials of different concentrations of PD and did not see a significant difference between coating thicknesses between samples. The characterization limitations of PD-based materials are often due to a lack of understanding in how the precise molecular mechanism behind PD formation functions (Battaglini, M., Emanet, M., Carmignani, A., Ciofani, G., 2024; Liebscher, J., Mrówczyński, R., Scheidt, H. A., Filip, C., Hǎdade, N. D., Turcu, R., Bende, A., Beck, S., 2013; and Ball, V., Hirtzel, J., Leks, G., Frisch, B., Talon, I, 2023).

### PD-Gent Coating Development and Validation

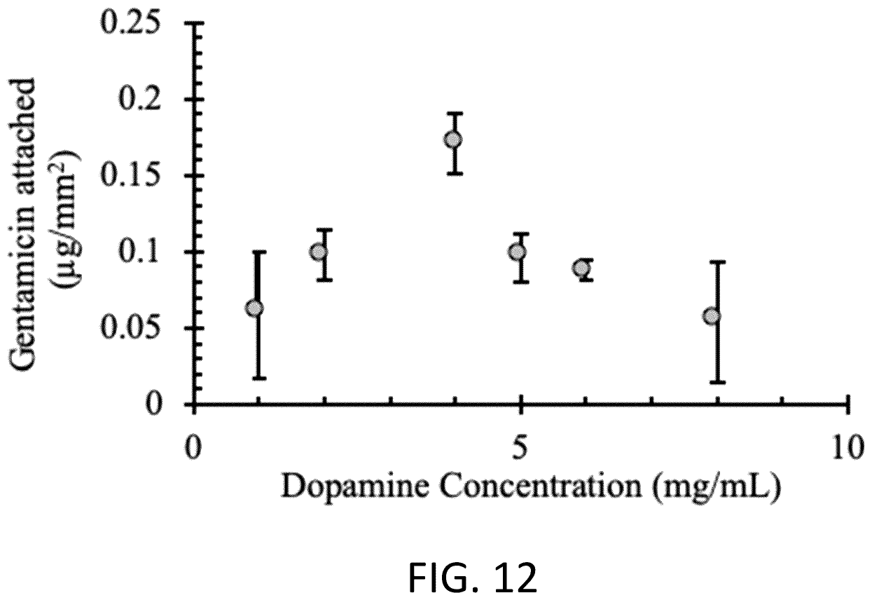

To determine the optimal PD concentration for this experimental design, we evaluated the effect of varying dopamine concentrations in the polymerization solution on bacterial viability. Dopamine hydrochloride was allowed to polymerize in the presence of gentamicin (2 mg/mL) and PVC tubes in Tris buffer at varying concentrations (1-10 mg/mL). This change in initial dopamine concentration controlled the gentamicin content in the resulting composite (FIG. 12). Liquid Chromatography Mass Spectroscopy (LCMS) was used to determine the concentration of gentamicin remaining in solution once the PVC tube was removed, which we used to calculate the amount of gentamicin incorporated in the composite coating.

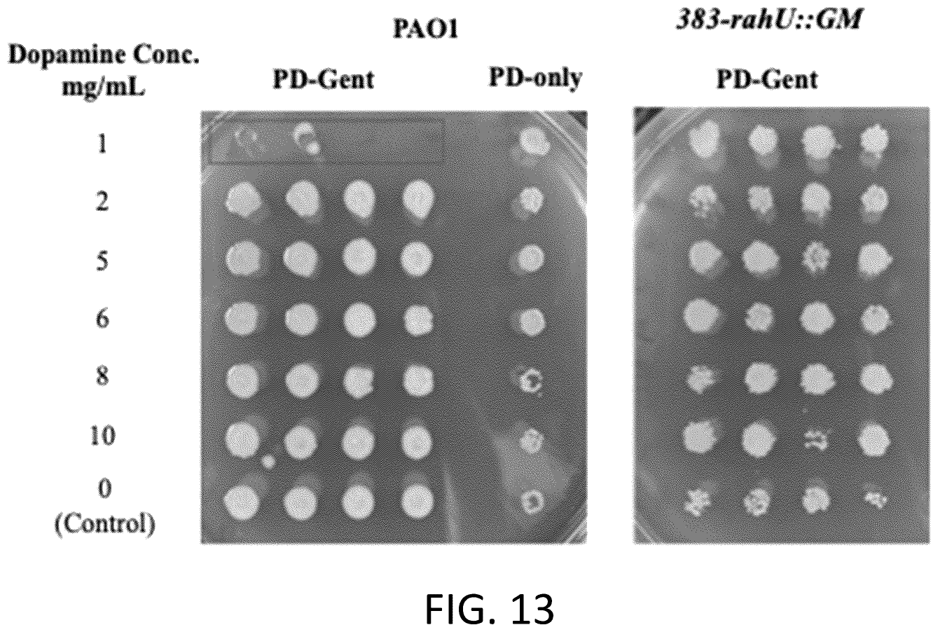

Gentamicin attachment steadily increased as the dopamine monomer concentration increased from 1 to 4 mg/mL; however, attachment systematically decreased when the polymerization was performed using 5-10 mg/mL of dopamine (FIG. 12). We initially hypothesized that coatings with the most gentamicin attachment (where PD concentration was 4 mg/mL), would result in the highest antimicrobial activity. Once gentamicin attachment was confirmed, the materials were further assessed for antibacterial activity using a laboratory strain of P. aeruginosa (PAO1) and a gentamicin-resistant strain, 383-rahU::GM. Despite the high gentamicin content, antibiotic release was unsuccessful at high dopamine coating solution concentrations. In fact, when PAO1 was exposed to the coating for 24 h and evaluated for bacterial viability, a bactericidal effect was observed using only 1.0 mg/mL dopamine hydrochloride (FIG. 13). We suspect at high concentrations of dopamine hydrochloride, the PD films were too thick, preventing gentamicin release. As expected, no bactericidal effect was observed when PAO1 was incubated with PVC tubes coated without gentamicin (PD-only). Similarly, we observed no bactericidal effect of PD-Gent against the gentamicin-resistant strain 383-rahU::GM (FIG. 13).

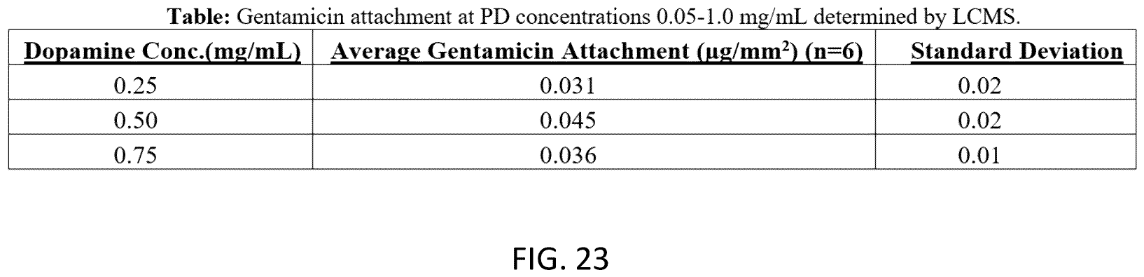

Based on these PAO1 growth data we synthesized coating composites using dopamine concentrations <1.0 mg/mL (0.25, 0.50, and 0.75 mg/mL), while maintaining a gentamicin concentration of 2 mg/mL in the polymerization solution. We observed negligible differences in gentamicin incorporation into the composite at these low PD concentrations (FIG. 23).

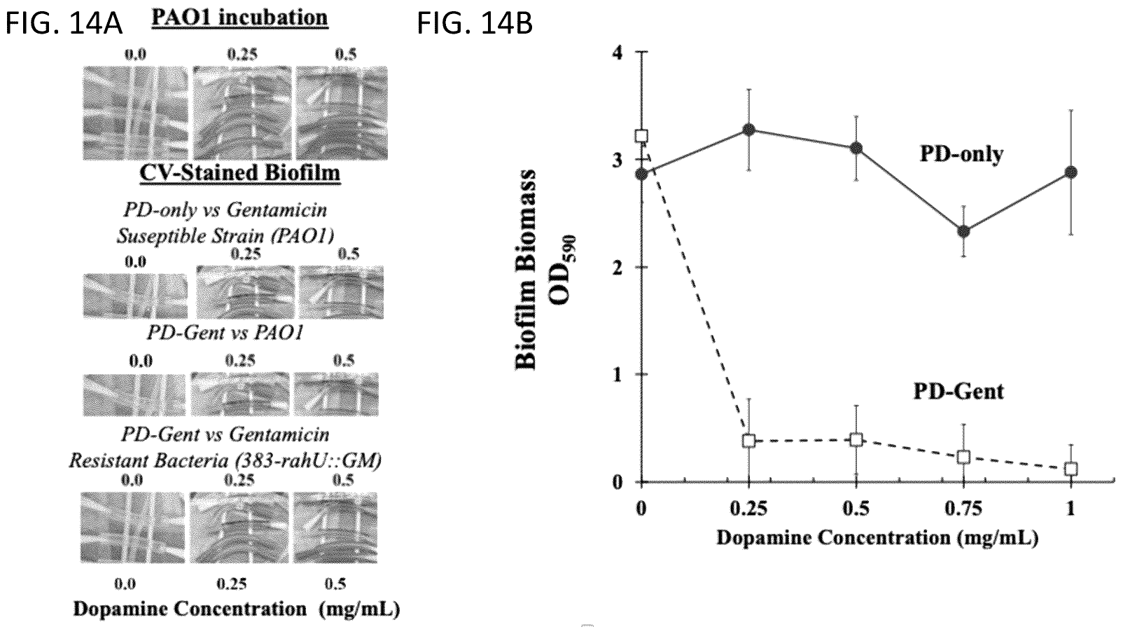

A standard concentration of PAO1 was inoculated into PVC tubes sealed with silicone stoppers and incubated for 24 h. The liquid content of each tube was removed from the sample and tubes were rinsed with DI water before adding 60 μL aliquots of CV, which were allowed to sit for 10 mins. Excess CV was rinsed out repeatedly using sterile water, CV-stained-tubes were air-dried overnight. PD-Gent coated PVC tubes showed minimal CV staining following PAO1 incubation, indicating effective inhibition of biofilm formation (FIG. 14A). In contrast, PD-only coated (made without gentamicin) and uncoated (control) tubes, retained dark purple CV staining consistent with substantial biofilm growth (FIG. 14A). There was a significant reduction in biofilm formation in PD-Gent samples synthesized with dopamine concentrations ranging from 0.25 to 1.0 mg/mL, compared to PD-only and uncoated control tubes (FIG. 14B). Gentamicin resistant strain (P. aeruginosa 383-rahU::GM) incubated in PD-Gent coated tubes retained their biofilm formation as expected.

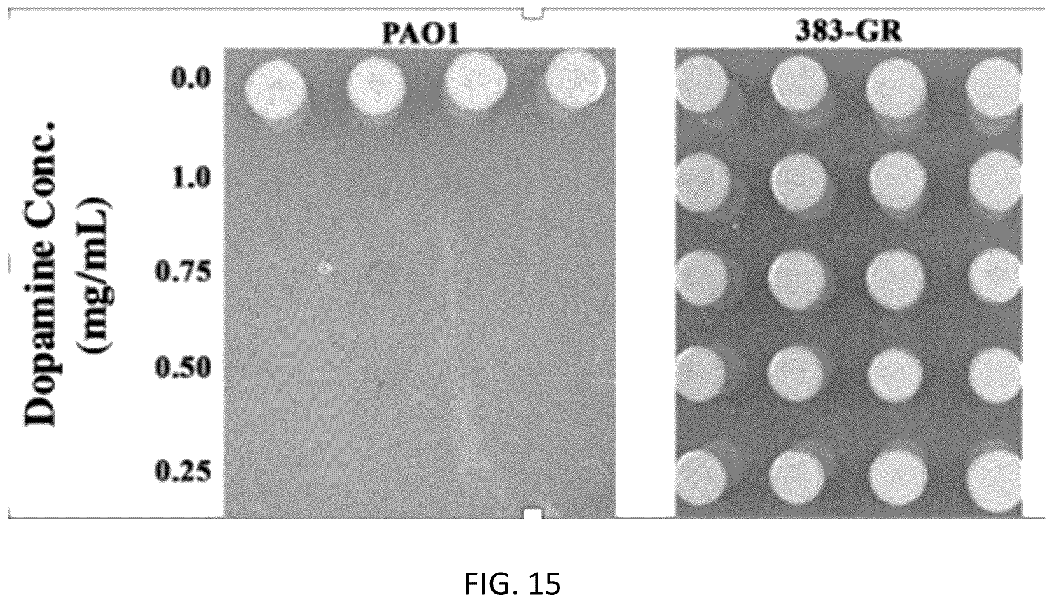

To evaluate bactericidal activity, the viability of PAO1 and 383-rahU::GM in the culture media was assessed following 24 hours of exposure to the PD-Gent coating (FIG. 15). The planktonic cell viability in the liquid contents of each tube was assessed qualitatively using a stamper. The liquid contents of the PD-Gent tubes showed no growth of PAO1 consistent across all concentrations of PD studied. As anticipated, there was no bactericidal effect observed in the gentamicin-resistant strain (FIG. 15).

### PD-Gent Coating Retains Antibiofilm Properties on Different Surfaces

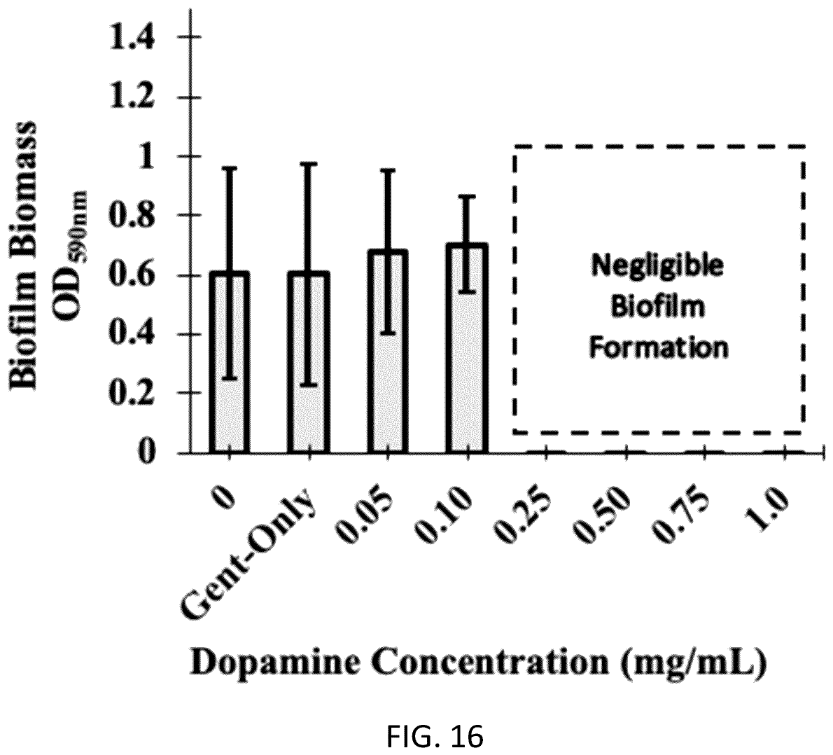

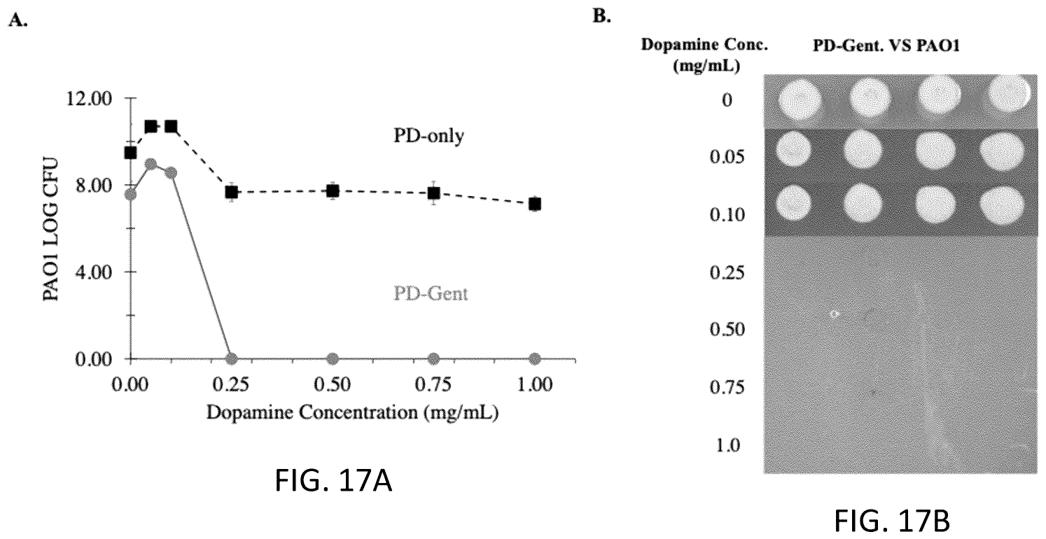

To determine if the coating material could adhere to other surfaces, we coated and assessed PAO1 biofilm formation and bacterial viability on polystyrene 96-well plates. PD-Gent coated 96-well plates at varying concentrations of PD were incubated with PAO1 for 24 h and were then assessed for biofilm formation and bacterial viability. The minimum PD content required for sufficient gentamicin attachment and antimicrobial activity was determined by varying the dopamine concentration from 0.05 mg/mL-1.0 mg/mL during coating synthesis. At dopamine concentrations 0.05 and 0.10 mg/mL, the biofilm formation remained similar to the uncoated-well suggesting insufficient polymer for effective gentamicin attachment (FIG. 16). However, at a dopamine concentration of 0.25 mg/mL, a reduction in biofilm formation was observed (FIG. 17A). As expected, no bactericidal effect was observed using the PD-only coating (FIG. 17A). There was no PAO1 cell viability observed for dopamine concentrations between 0.25-1.0 mg/mL (FIG. 17B). Similarly, PD-Gent coatings with PD concentrations between 0.25-1 mg/mL also inhibited biofilm growth (FIG. 16). These results are congruent with the reduction in PAO1 biofilm and cell viability observed in PD-Gent-coated PVC tubes. A control was performed where PS plates were coated with a gentamicin solution (0 mg/mL PD) for 24 h at 60° C., followed by rinsing with DI water. In the absence of PD (Gent-only), biofilm formation was comparable to that observed in uncoated (control) wells (FIG. 16), indicating that gentamicin does not effectively bind without the presence of adequate PD.

### PD-Gent Coating Methodology is Versatile

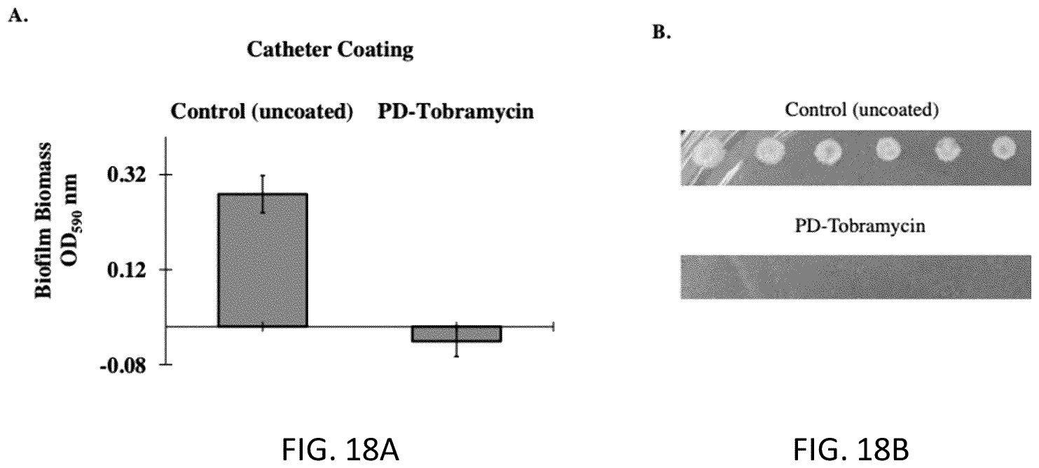

Tobramycin is another aminoglycoside antibiotic that is effective against gram-negative bacteria. To evaluate the versatility of our coating methodology, PD composite coatings well-plates were coated in a PD-Tobramycin conjugate using a similar procedure described for the PD-Gent coating, substituting tobramycin for gentamicin. As with the PD-Gent plates, we observed successful biofilm inhibition using the PD-Tobramycin coating compared to uncoated wells (FIG. 18A). The growth of planktonic bacteria was also successfully inhibited, indicating that the PD-antibiotic protocol is applicable with multiple antibiotics (FIG. 18B).

### In Vitro Artificial Urinary Tract Model

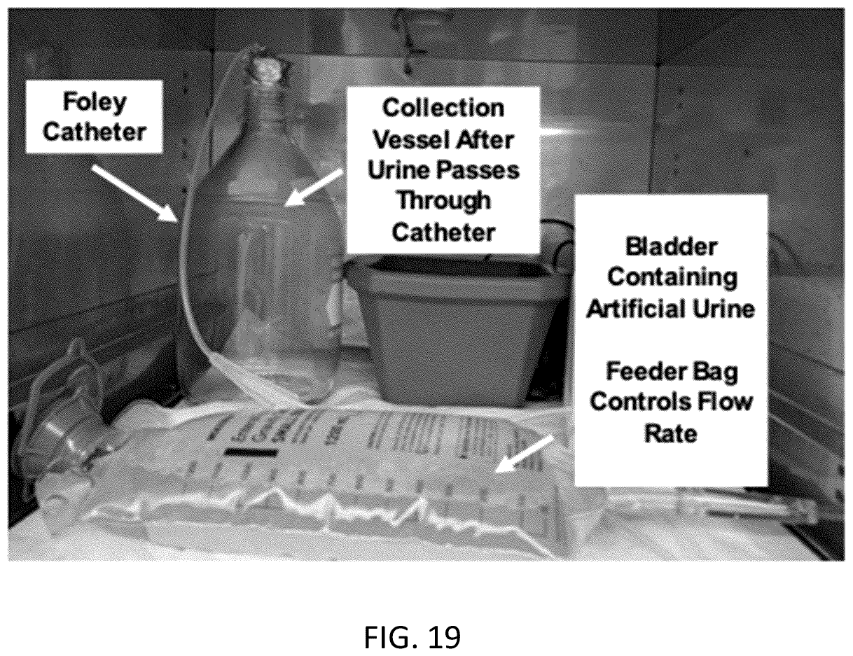

Previous bacterial incubation in PVC tubes assessed biofilm growth under static conditions. To more accurately simulate patient conditions, an experimental model was designed to incorporate urinary catheters subjected to constant flow of bacteriuria (FIG. 19). In this system, Bardex® Foley silicone catheters were connected to a sterile reservoir simulating a bladder, in this case comprised of a feeding bag. This flow was controlled by a roller clamp on the feeding bag, which delivered the bacteriuria at a constant rate (approximately 0.1 mL/min). To maintain continuous flow and prevent uncontrolled PAO1 growth, the bag was elevated outside the incubator at room temperature. Meanwhile, the flow inside the catheter occurred within the incubator, creating a warm environment conducive to biofilm formation, analogous to internal body conditions.

After 24 h of bacterial flow, the PD-Gent coated catheter and uncoated control catheter were stained with CV to assess biofilm biomass. Limited biofilm was observed in the PD-Gent coated catheter. Biofilm was then solubilized from each catheter using 5 mL of 33% glacial acetic acid, producing a biofilm stock solution.

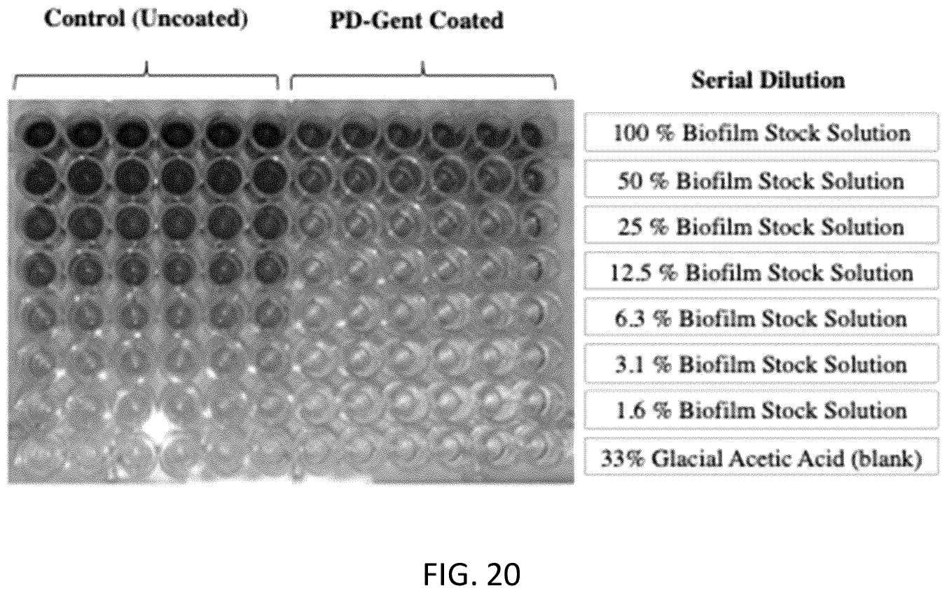

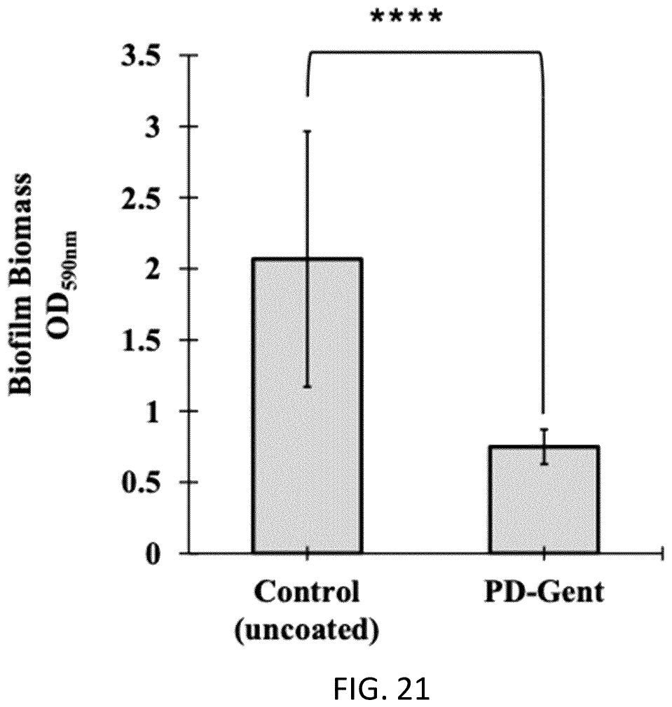

The initial biofilm stock solution from uncoated control and PD-gent coated catheters were oversaturated, exceeding the detection range of the spectrophotometer and preventing accurate optical density measurements. This stock solution was serially diluted to optimize detection of optical (OD₅₉₀nm) during spectrophotometric analysis (FIG. 20). Following 1:1 dilution of the biofilm stock, the spectrophotometer was able to detect a three-fold reduction in biofilm mass for the PD-Gent coated catheter compared to the uncoated control (FIG. 21).

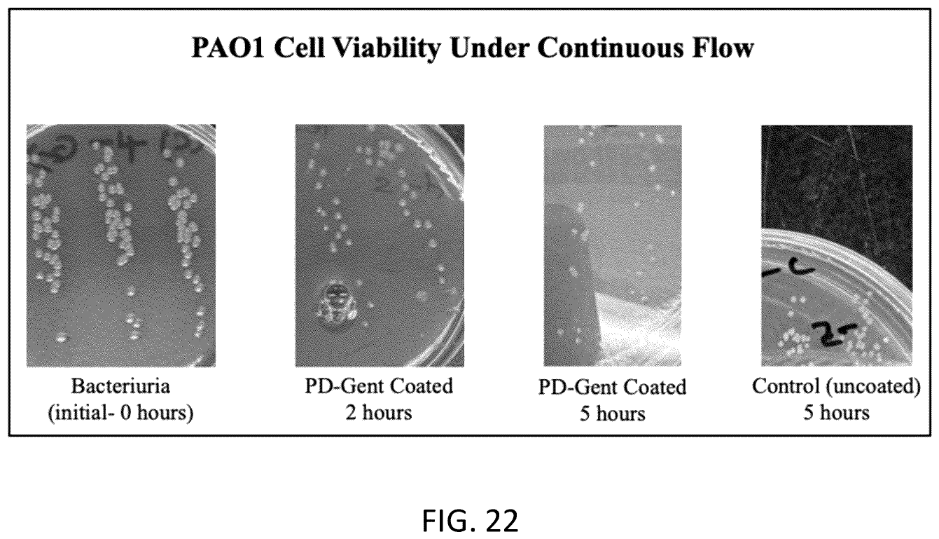

As anticipated, greater biofilm formation occurred in the PD-Gent coated catheters under flow conditions compared to static conditions, likely due to the continuous introduction of bacteria. Nevertheless, PD-Gent coated Foley catheters significantly inhibited biofilm formation relative to the uncoated control catheters (FIG. 22). The in vitro bladder experiment was conducted in triplicate using three independent biological replicates. The statistical significance of biofilm biomass was determined using a two-tailed t-test where the p-value was 0.0000011. To assess planktonic bacterial viability in coated and uncoated catheters, 30 μL aliquots of the bacteriuria were collected from the catheter outlet over time and plated on agar, followed by overnight incubation at 37° C. A significant reduction in surviving PAO1 colonies was observed after 2 and 5 h of continuous flow and exposure to PD-Gent (FIG. 22). While the degree of reductions in planktonic PAO1 viability varied across the three independent trials (FIG. 22), the inhibition of biofilm formation remained consistent. In vitro flow experiments were also conducted using pre-formed biofilm solutions, where PAO1 was allowed to grow for 48 h. In these experiments PD-Gent coated catheters were successfully able to inhibit growth of PAO1 planktonic bacteria, but unable to prevent biofilm growth on catheter surface. This finding was unsurprising since biofilms are typically recalcitrant to antibiotics and the preformed biofilm likely attached onto the catheter surface (Lebeaux, D., Ghigo, J. M., Beloin, C., 2014; and Verderosa, A. D., Totsika, M., Fairfull-Smith, K. E, 2019).

### Conclusion

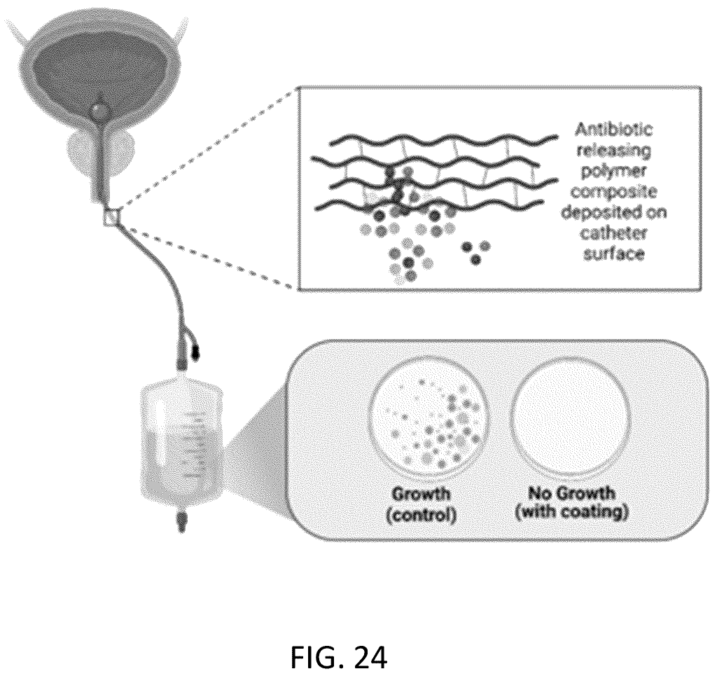

Antibiofilm and antimicrobial coating methodology was developed for catheter surfaces. Polydopamine was used to anchor antimicrobial agents (gentamicin or tobramycin) to the surface of PVC tubes, polystyrene plates, and urinary catheters. By leveraging the adhesive properties of polydopamine and the potent antibacterial activity of gentamicin, we created a surface-modified coating capable of significantly reducing P. aeruginosa biofilm accumulation under both static and dynamic flow conditions. Notably, this work establishes a tunable range of coating polymerization conditions that achieves antibiofilm and antimicrobial effects. The PD composite has compatibility with other antibiotics (such as tobramycin), indicating this treatment mechanism is adaptable. Although the long-term durability of the coating remains unknown, this approach offers a clinically relevant, scalable solution for preventing biofilm-related infections on indwelling medical devices. Such concept is shown in the diagram of FIG. 24 with respect to a urinary catheter. The PD-Gent coating platform demonstrates broad surface compatibility (i.e., PVC, PS, silicone), enabling its integration into diverse medical devices beyond urinary catheters, and thus holds significant promise for preventing biofilm-associated infections across a wide spectrum of clinical applications.

The present disclosure has described particular implementations having various features. In light of the disclosure provided above, it will be apparent to those skilled in the art that various modifications and variations can be made without departing from the scope or spirit of the disclosure. One of ordinary skill in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

### Supplementary Sequence Information

FIGS. 25-31 provide additional sequence information for example PNAs of the disclosure in the following order:
PNA Name (in bold)
Target Organism; Genomic Accession No.
PNA Location
PNA Sequence and Reverse Complement Sequence
Gene Information
Gene Location
Inclusive Range of the Genomic Target Sequence

The Genomic Target Sequence (with PNA Sequence highlight in yellow with the start codon in bold).

### REFERENCES

Abes R., Moulton H.M., Clair P., Yang S.T., Abes S., Melikov K., Prevot P., Youngblood D.S., Iversen P.L., Chernomordik L.V., Lebleu B., 2008. Delivery of steric block morpholino oligomers by (R-X-R)4 peptides: structure-activity studies. Nucleic Acids Res. 36(20), 6343-54. DOI: 10.1093/nar/gkn541.

Adetunji, V., 2011. Crystal violet binding assay for assessment of biofilm formation by isolates of Listeria monocytogenes and Listeria spp from a typical tropical abattoir on wood, steel and glass surfaces. Global Veterinaria 6, 6-10.

AHRQ's Healthcare-Associated Infections Program [WWW Document], n.d. URL https://www.ahrq.gov/hai/index.html (accessed 11.17.23).

Al-Qahtani, M.; Safan, A.; Jassim, G.; Abadla, S, 2019. Efficacy of anti-microbial catheters in preventing catheter associated urinary tract infections in hospitalized patients: A review on recent updates. Journal of Infection and Public Health, 12 (6), 760-766.

Arciola, C.R., Campoccia, D., Montanaro, L., 2018. Implant infections: adhesion, biofilm formation and immune evasion. Nat Rev Microbiol 16, 397-409. DOI: 10.1038/s41579-018-0019-y.

Armbruster, C. E., Hodges, S. A. & Mobley, H. L. T, 2013. Initiation of swarming motility by Proteus mirabilis occurs in response to specific cues present in urine and requires excess L-glutamine. J Bacteriol 195, 1305-131.

Assadi, F, 2018. Strategies for preventing catheter-associated urinary tract infections. Int J Prev Med 9.

Assefa, M. & Amare, A., 2022. Biofilm-Associated Multi-Drug Resistance in Hospital-Acquired Infections: A Review. Infect Drug Resist 15, 5061-5068.

Ball, V.; Hirtzel, J.; Leks, G.; Frisch, B.; Talon, I, 2023. Experimental Methods to Get Polydopamine Films: A Comparative Review on the Synthesis Methods, the Films' Composition and Properties. Macromolecular Rapid Communications 44 (16), 2200946. DOI: 10.1002/marc.202200946.

Barkowsky, G., Lemster, A.-L., Pappesch, R., Jacob, A., Krüger, S., Schröder, A., Kreikemeyer, B., Patenge, N., 2019. Influence of Different Cell-Penetrating Peptides on the Antimicrobial Efficiency of PNAs in Streptococcus pyogenes. Mol Ther Nucleic Acids 18, 444-454. DOI: 10.1016/j.omtn.2019.09.010.

Battaglini, M.; Emanet, M.; Carmignani, A.; Ciofani, G, 2024. Polydopamine-based nanostructures: A new generation of versatile, multi-tasking, and smart theranostic tools. Nano Today 55, 102151.

Batul, R.; Bhave, M.; Yu, A, 2023. Investigation of Antimicrobial Effects of Polydopamine-Based Composite Coatings. Molecules. 28 (11). DOI: 10.3390/molecules28114258.

Ciarolla, A.A., Lapin, N., Williams, D., Chopra, R., Greenberg, D.E., 2022. Physical Approaches to Prevent and Treat Bacterial Biofilm. Antibiotics (Basel) 12, 54. DOI: 10.3390/antibiotics12010054.

Chen, N.; Yao, S.; Li, M.; Wang, Q.; Sun, X.; Feng, X.; Chen, Y., 2023. Nonporous versus Mesoporous Bioinspired Polydopamine Nanoparticles for Skin Drug Delivery. Biomacromolecules 24 (4), 1648-1661. DOI: 10.1021/acs.biomac.2c01431.

Cole, S. J.; Records, A. R.; Orr, M. W.; Linden, S. B.; Lee, V. T., 2014. Catheter-associated urinary tract infection by Pseudomonas aeruginosa is mediated by exopolysaccharide-independent biofilms. Infect Immun. 82 (5), 2048-2058. DOI: 10.1128/iai.01652-14.

Davidsen, M. B.; Teixeira, J. F. L.; Dehli, J.; Karlsson, C.; Kraft, D.; Souza, P. P. C.; Foss, M., 2021. Post-treatments of polydopamine coatings influence cellular response. Colloids and Surfaces B: Biointerfaces 207, 111972. DOI: 10.1016/j.colsurfb.2021.111972.

Donlan, R.M., Costerton, J.W., 2002. Biofilms: survival mechanisms of clinically relevant microorganisms. Clin Microbiol Rev 15, 167-193. DOI: 10.1128/CMR.15.2.167-193.2002.

Dubern, J.-F. et al., 2023. Discovery of a polymer resistant to bacterial biofilm, swarming, and encrustation. Sci Adv 9.

Eze, E.C., Chenia, H.Y., El Zowalaty, M.E., 2018. Acinetobacter baumannii biofilms: effects of physicochemical factors, virulence, antibiotic resistance determinants, gene regulation, and future antimicrobial treatments. Infect Drug Resist 11, 2277-2299. DOI: 10.2147/IDR.S169894.

Flemming, H.-C., Wingender, J., Szewzyk, U., Steinberg, P., Rice, S.A., Kjelleberg, S., 2016. Biofilms: an emergent form of bacterial life. Nat Rev Microbiol 14, 563-575. DOI: 10.1038/nrmicro.2016.94.

Flores-Mireles, A.; Hreha, T. N.; Hunstad, D. A., 2019, Pathophysiology, Treatment, and Prevention of Catheter-Associated Urinary Tract Infection. (1945-5763 (Electronic)).

Fu, Y.; Yang, L.; Zhang, J.; Hu, J.; Duan, G.; Liu, X.; Li, Y.; Gu, Z., 2021. Polydopamine antibacterial materials. Materials Horizons 8 (6), 1618-1633, DOI: 10.1039/DOMH01985B.

Gambrill, B.; Pertusati, F.; Hughes, S. F.; Shergill, I.; Prokopovich, P., 2024., Materials-based incidence of urinary catheter associated urinary tract infections and the causative micro-organisms: systematic review and meta-analysis. BMC Urology 24 (1). DOI: 10.1186/s12894-024-01565-x.

Goda, R. M.; El-Baz, A. A.-O.; Khalaf, E. A.-O.; Alharbi, N. A.-O.; Elkhooly, T. A.-O.; Shohayeb, M. M., 2022. Combating Bacterial Biofilm Formation in Urinary Catheter by Green Silver Nanoparticle. LID-10.3390/antibiotics11040495 [doi] LID-495. Antibiotics 11 (495).

Grossman, A. B.; Burgin, D. J.; Rice, K. C., 2021. Quantification of Staphylococcus aureusStaphylococcus aureusBiofilmBiofilmsFormation by Crystal VioletCrystal violet and Confocal MicroscopyConfocal microscopy. In Staphylococcus aureus: Methods and Protocols, Rice, K. C. Ed.; Springer US, 69-78.

Harbarth, S.; Rohner, P.; Safran, E.; Garbino, J.; Auckenthaler, R.; Pittet, D, 1998. Resistance to amikacin and gentamicin among Gram-negative bloodstream isolates in a university hospital between 1989 and 1994. Clinical Microbiology and Infection. 4 (4), 199-204. DOI: 10.1111/j.1469-0691.1998.tb00669.x.

Hemmatpour, H.; De Luca, O.; Crestani, D.; Stuart, M. C. A.; Lasorsa, A.; Van Der Wel, P. C. A.; Loos, K.; Giousis, T.; Haddadi-Asl, V.; Rudolf, P., 2023. New insights in polydopamine formation via surface adsorption. Nature Communications 14 (1). DOI: 10.1038/s41467-023-36303-8.

Hollenbeak, C. & Schilling, A., 2018. The attributable cost of catheter-associated urinary tract infections in the United States: A systematic review. Am J Infect Control 46, 751-757.

Hu, J., Xia, Y., Xiong, Y., Li, X. & Su, X., 2011 Inhibition of biofilm formation by the antisense peptide nucleic acids targeted at the motA gene in Pseudomonas aeruginosa PAO1 strain. World J Microbiol Biotechnol 27, 1981-1987.

Jones, C. J., Ryder, C. R., Mann, E. E. & Wozniak, D. J., 2013. AmrZ modulates pseudomonas aeruginosa biofilm architecture by directly repressing transcription of the psl operon. J Bacteriol 195, 1637-1644.

Kamimura, R.; Kanematsu, H.; Ogawa, A.; Kogo, T.; Miura, H.; Kawai, R.; Hirai, N.; Kato, T.; Yoshitake, M.; Barry, D. M., 2022. Quantitative Analyses of Biofilm by Using Crystal Violet Staining and Optical Reflection. Materials (Basel) 15 (19). DOI: 10.3390/ma15196727.

Karmarkar, R.; Bodapati, S.; Yao, L.; Aroori, S. A., 2024. Foley catheter ‘the jack of all trades’: a literature review of its common and novel uses. Ann R Coll Surg Engl 106 (6), 485-491. DOI: 10.1308/rcsann.2023.0003.

Kemnic, T. R. & Gulick, P. G., 2022. HIV Antiretroviral Therapy Continuing Education Activity. NCBI Bookshelf https://www.ncbi.nlm.nih.gov/books/NBK513308/.

Lebeaux, D., Ghigo, J.-M., Beloin, C., 2014. Biofilm-related infections: bridging the gap between clinical management and fundamental aspects of recalcitrance toward antibiotics. Microbiol Mol Biol Rev 78, 510-543. DOI: 10.1128/MMBR.00013-14.

Li, Y.; Hong, W.; Zhang, H.; Zhang, T. T.; Chen, Z.; Yuan, S.; Peng, P.; Xiao, M.; Xu, L., 2020. Photothermally triggered cytosolic drug delivery of glucose functionalized polydopamine nanoparticles in response to tumor microenvironment for the GLUT1-targeting chemo-phototherapy. Journal of Controlled Release, 317, 232-245.

Liebscher, J.; Mrówczyński, R.; Scheidt, H. A.; Filip, C.; Hǎdade, N. D.; Turcu, R.; Bende, A.; Beck, S., 2013. Structure of Polydopamine: A Never-Ending Story? Langmuir 29 (33), 10539-10548. DOI: 10.1021/1a4020288.

Lila, A. S. A. et al., 2023. Biofilm Lifestyle in Recurrent Urinary Tract Infections. Life 13.

Lim, K.; Chua, R. R. Y.; Ho, B.; Tambyah, P. A.; Hadinoto, K.; Leong, S. S. J., 2015. Development of a catheter functionalized by a polydopamine peptide coating with antimicrobial and antibiofilm properties. Acta Biomaterialia 15, 127-138. DOI: 10.1016/j.actbio.2014.12.015.

Louden, B. C., Haarmann, D. & Lynne, A. M., 2011. Use of Blue Agar CAS Assay for Siderophore Detection. J Microbiol Biol Educ 12, 51-53.

Ma, K.; Dong, P.; Liang, M.; Yu, S.; Chen, Y.; Wang, F., 2020. Facile Assembly of Multifunctional Antibacterial Nanoplatform Leveraging Synergistic Sensitization between Silver Nanostructure and Vancomycin. ACS Applied Materials & Interfaces 12 (6), 6955-6965. DOI: 10.1021/acsami.9b22043.

Magill, S.S., Edwards, J.R., Bamberg, W., Beldavs, Z.G., Dumyati, G., Kainer, M.A., Lynfield, R., Maloney, M., McAllister-Hollod, L., Nadle, J., Ray, S.M., Thompson, D.L., Wilson, L.E., Fridkin, S.K., 2014. Multistate Point-Prevalence Survey of Health Care-Associated Infections. N Engl J Med 370, 1198-1208. DOI: 10.1056/NEJMoa1306801.

Menchise, V. et al., 2003. Insights into peptide nucleic acid (PNA) structural features: The crystal structure of a D-lysine-based chiral PNA-DNA duplex FIG. 1. Chemical structures of PNA and DNA oligomers. PNAS 14, 12021-12026.

Mendhe, S.; Badge, A.; Ugemuge, S.; Chandi, D, 2023. Impact of Biofilms on Chronic Infections and Medical Challenges. Cureus 15 (11), e48204. DOI: 10.7759/cureus.48204.

Merritt, J. H.; Kadouri, D. E.; O'Toole, G. A., 2005. Growing and analyzing static biofilms. Curr Protoc Microbiol, Chapter 1. Unit 1B.1. DOI: 10.1002/9780471729259.mc01b01s00.

Michalicha, A., Palka, K., Roguska, A., Pisarek, M., Belcarz, A., 2021. Polydopamine-coated curdlan hydrogel as a potential carrier of free amino group-containing molecules. Carbohydrate Polymers, Volume 256, 117524, DOI: 10.1016/j.carbpol.2020.117524.

Mika, F. & Hengge, R., 2014. Small RNAs in the control of RpoS, CsgD, and biofilm architecture of Escherichia coli. RNA Biol 11, 494-507.

Muhammad, M. H. et al., 2020. Beyond Risk: Bacterial Biofilms and Their Regulating Approaches. Front Microbiol 11.

Murari, G.; Bock, N.; Zhou, H.; Yang, L.; Liew, T.; Fox, K.; Tran, P. A., 2020. Effects of polydopamine coatings on nucleation modes of surface mineralization from simulated body fluid. Scientific Reports 10 (1), 14982. DOI: 10.1038/s41598-020-71900-3.

Narenji, H. et al., 2017. Peptide nucleic acids (PNAs): currently potential bactericidal agents. Biomedicine and Pharmacotherapy 93, 580-588.

Necula, B. S.; van Leeuwen, J. P. T. M.; Fratila-Apachitei, L. E.; Zaat, S. A. J.; Apachitei, I.; Duszczyk, J., 2012. In vitro cytotoxicity evaluation of porous TiO2-Ag antibacterial coatings for human fetal osteoblasts. Acta Biomaterialia 8 (11), 4191-4197.

Nicolle, L. E., 2014. Catheter associated urinary tract infections. Antimicrobial Resistance & Infection Control 3 (23), 2047-2994.

Nielsen, P.E., Egholm, M., Berg, R.H., Buchardt, O., 1991. Sequence-selective recognition of DNA by strand displacement with a thymine-substituted polyamide. Science 254, 1497-1500. DOI: 10.1126/science. 1962210.

Nye, T. M. et al., 2024. Microbial co-occurrences on catheters from long-term catheterized patients. Nat Commun 15.

Orazi, G., O'Toole, G.A., 2019. “It Takes a Village”: Mechanisms Underlying Antimicrobial Recalcitrance of Polymicrobial Biofilms. J Bacteriol 202. DOI: 10.1128/JB.00530-19.

Pellestor, F., Paulasova, P., 2004. The peptide nucleic acids (PNAs), powerful tools for molecular genetics and cytogenetics. Eur J Hum Genet 12, 694-700. DOI: 10.1038/sj.ejhg.5201226.

Pickard, R. et al., 2012. Types of urethral catheter for reducing symptomatic urinary tract infections in hospitalised adults requiring short-term catheterisation: Multicentre randomised controlled trial and economic evaluation of antimicrobial- and antisepticimpregnated urethral catheters (the CATHETER trial). Health Technol Assess (Rockv) 16.

PNA Bio. Custom PNA Oligos., 2022. https://www.pnabio.com/products/PNA_oligo.htm.

Preda, V. G.; Sǎndulescu, O., 2019. Communication is the key: biofilms, quorum sensing, formation and prevention. Discoveries 7 (3), 2359-7232.

Rajasekaran, P. et al., 2013. Peptide nucleic acids inhibit growth of Brucella suis in pure culture and in infected murine macrophages. Int J Antimicrob Agents 41, 358-362.

Rao, J. et al., 2011. Host derived inflammatory phospholipids regulate rahU (PA0122) Gene, Protein, and Biofilm Formation in Pseudomonas aeruginosa. Cell Immunol 270, 95-102.

Ren, D.; Williams, G. R.; Zhang, Y.; Ren, R.; Lou, J.; Zhu, L.-M., 2022. Mesoporous Doxorubicin-Loaded Polydopamine Nanoparticles Coated with a Platelet Membrane Suppress Tumor Growth in a Murine Model of Human Breast Cancer. ACS Applied Bio Materials 5 (1), 123-133. DOI: 10.1021/acsabm.1c00926.

Roy, R., Tiwari, M., Donelli, G., Tiwari, V., 2018. Strategies for combating bacterial biofilms: A focus on anti-biofilm agents and their mechanisms of action. Virulence 9, 522-554. DOI: 10.1080/21505594.2017.1313372.

Rukholm, G.; Mugabe C Fau-Azghani, A. O.; Azghani Ao Fau-Omri, A.; Omri, A., 2006. Antibacterial activity of liposomal gentamicin against Pseudomonas aeruginosa: a time-kill study. (0924-8579 (Print)).

Sarigul, N.; Korkmaz, F.; Kurultak, İ., 2019. A New Artificial Urine Protocol to Better Imitate Human Urine. Scientific Reports 9 (1), 20159. DOI: 10.1038/s41598-019-56693-4.

Sharma, S., Mohler, J., Mahajan, S.D., Schwartz, S.A., Bruggemann, L., Aalinkeel, R., 2023. Microbial Biofilm: A Review on Formation, Infection, Antibiotic Resistance, Control Measures, and Innovative Treatment. Microorganisms 11, 1614. DOI: 10.3390/microorganisms11061614.

Sharma, D., Misba, L. & Khan, A. U., 2019. Antibiotics versus biofilm: An emerging battleground in microbial communities. Antimicrob Resist Infect Control 8.

Singh, I., Priyam, A, Jha, D, Dhawan, G, Gautam, H.K., Kumar, P., 2020. Polydopamine-aminoglycoside nanoconjugates: Synthesis, characterization, antimicrobial evaluation and cytocompatibility, Materials Science and Engineering: C, Volume 107, 110284, DOI: 10.1016/j.msec.2019.110284.

Srinivasan, R., Santhakumari, S., Poonguzhali, P., Geetha, M., Dyavaiah, M., Xiangmin, L., 2021. Bacterial Biofilm Inhibition: A Focused Review on Recent Therapeutic Strategies for Combating the Biofilm Mediated Infections. Front. Microbiol. 12. DOI: 10.3389/fmicb.2021.676458.

Stewart, P.S., Costerton, J.W., 2001. Antibiotic resistance of bacteria in biofilms. Lancet 358, 135-138. DOI: 10.1016/s0140-6736(01)05321-1.

Stickler, D. J., 2014. Clinical complications of urinary catheters caused by crystalline biofilms: Something needs to be done. J Intern Med 276, 120-129.

Su, Y. et al., 2022 Biofilms: Formation, Research Models, Potential Targets, and Methods for Prevention and Treatment. Advanced Science 9.

Swenson, C. S. & Heemstra, J. M., 2012. Peptide Nucleic Acids Harness Dual Information Codes in a Single Molecule. Chem Comm DOI: 10.1039/x0xx00000x.

Tuon, F.F., Dantas, L.R., Suss, P.H., Tasca Ribeiro, V.S., 2022. Pathogenesis of the Pseudomonas aeruginosa Biofilm: A Review. Pathogens 11, 300. DOI: 10.3390/pathogens11030300.

U.S. Centers for Disease Control and Prevention, 2024. Catheter-associated Urinary Tract Infection Basics. https://www.cdc.gov/uti/about/cauti-basics.html?CDC_AAref_Val=https://www.cdc.gov/hai/ca_uti/uti.html.

Vakulskas, C. A., Potts, A. H., Babitzke, P., Ahmer, B. M. M. & Romeo, T., 2015. Regulation of Bacterial Virulence by Csr (Rsm) Systems. Microbiology and Molecular Biology Reviews 79, 193-224.

Vazquez-Rodriguez, J. A.; Shaqour, B.; Guarch-Pérez, C.; Choińska, E.; Riool, M.; Verleije, B.; Beyers, K.; Costantini, V. J. A.; Świȩszkowski, W.; Zaat, S. A. J.; et al., 2022. A Niclosamide-releasing hot-melt extruded catheter prevents Staphylococcus aureus experimental biomaterial-associated infection. Scientific Reports 12 (1), 12329. DOI: 10.1038/s41598-022-16107-4.

Verderosa, A. D.; Totsika, M.; Fairfull-Smith, K. E., 2019. Bacterial Biofilm Eradication Agents: A Current Review. Frontiers in Chemistry 7. DOI: 10.3389/fchem.2019.00824.

Vertes, A.; Hitchins, V.; Phillips, K. S., 2012. Analytical Challenges of Microbial Biofilms on Medical Devices. Analytical Chemistry 84 (9), 3858-3866. DOI: 10.1021/ac2029997.

Wang, B.; Yuan, T.; Zha, L.; Liu, Y.; Chen, W.; Zhang, C.; Bao, Y.; Dong, Q., 2021. Oral Delivery of Gambogenic Acid by Functional Polydopamine Nanoparticles for Targeted Tumor Therapy. Molecular Pharmaceutics 18 (3), 1470-1479. DOI: 10.1021/acs.molpharmaceut.1c00030.

Weiner-Lastinger, L.M., Abner, S., Edwards, J.R., Kallen, A.J., Karlsson, M., Magill, S.S., Pollock, D., See, I., Soe, M.M., Walters, M.S., Dudeck, M.A., 2020. Antimicrobial-resistant pathogens associated with adult healthcare-associated infections: Summary of data reported to the National Healthcare Safety Network, 2015-2017. Infect Control Hosp Epidemiol 41, 1-18. DOI: 10.1017/ice.2019.296.

Williamson, K.S., Richards, L.A., Perez-Osorio, A.C., Pitts, B., McInnerney, K., Stewart, P.S., Franklin, M.J., 2012. Heterogeneity in Pseudomonas aeruginosa Biofilms Includes Expression of Ribosome Hibernation Factors in the Antibiotic-Tolerant Subpopulation and Hypoxia-Induced Stress Response in the Metabolically Active Population. J Bacteriol 194, 2062-2073. DOI: 10.1128/JB.00022-12.

Wilson, C. et al., 2017. Quantitative and Qualitative Assessment Methods for Biofilm Growth: A Mini-review HHS Public Access. Res Rev J Eng Technol 6.

Xu, Z.; Liang, Y.; Lin, S.; Chen, D.; Li, B.; Li, L.; Deng, Y., 2016. Crystal Violet and XTT Assays on Staphylococcus aureus Biofilm Quantification. Current Microbiology 73 (4), 474-482. DOI: 10.1007/s00284-016-1081-1.

Yang, W.; Liu, C.; Chen, Y., 2018. Stability of Polydopamine Coatings on Gold Substrates Inspected by Surface Plasmon Resonance Imaging. Langmuir 34 (12), 3565-3571. DOI: 10.1021/acs.langmuir.7b03143.

Zafer, M. M.; Mohamed, G. A.; Ibrahim, S. R. M.; Ghosh, S.; Bornman, C.; Elfaky, M. A., 2024. Biofilm-mediated infections by multidrug-resistant microbes: a comprehensive exploration and forward perspectives. Archives of Microbiology 206 (3), 101. DOI: 10.1007/s00203-023-03826-z.

Zegans, M. E.; Wagner, J. C.; Cady, K. C.; Murphy, D. M.; Hammond, J. H.; O'Toole, G. A., 2009. Interaction between bacteriophage DMS3 and host CRISPR region inhibits group behaviors of Pseudomonas aeruginosa. J Bacteriol 191 (1), 210-219. DOI: 10.1128/jb.00797-08.

1. A peptide nucleic acid or combination of peptide nucleic acids, wherein each peptide nucleic acid comprises a nucleobase sequence that has at least about 80%, 85%, 90%, 95%, 96%, 97%, 98% and 99% sequence identity to a nucleobase sequence chosen from SEQ ID NOS:1-4 and 6-10, or any reverse, reverse complementary, or complementary sequence thereof.

2. The peptide nucleic acid or combination of peptide nucleic acids of claim 1, wherein the PNA or one or more of the PNAs of the combination comprises a nucleobase sequence chosen from SEQ ID NOS:1-4 and 6-10, or any reverse, reverse complementary, or complementary sequence thereof.

3. The peptide nucleic acid or combination of peptide nucleic acids of claim 1, wherein the PNA or one or more of the PNAs of the combination is linked or conjugated to a cell penetrating peptide.

4. The peptide nucleic acid or combination of peptide nucleic acids of claim 2, wherein the cell penetrating peptide is a peptide comprising an amino acid sequence chosen from SEQ ID NO:11, SEQ ID NO:12, or SEQ ID NO:13.

5. A pharmaceutical composition comprising the peptide nucleic acid or combination of peptide nucleic acids of claim 1 within a pharmaceutical carrier.

6. The pharmaceutical composition of claim 5, wherein the pharmaceutical carrier is a solution, suspension, emulsion, ointment, cream, or gel.

7. The pharmaceutical composition of claim 5, wherein the pharmaceutical carrier is or includes one or more polymer.

8. The pharmaceutical composition of claim 7, wherein the one or more polymer is chosen from one or more of a polyester, a polyurethane, a poly(meth) acrylate, a polysaccharide, a polyamide, a polynorbornene, a polycarbonate, a poly(meth) acrylamide, a polyoxazoline, a poly(ethylene oxide), a polyaziridine, or a polysiloxane.

9. The pharmaceutical composition of claim 5, wherein the pharmaceutical carrier is or includes polydopamine.

10. The pharmaceutical composition of claim 5, further comprising one or more antibiotics.

11. The pharmaceutical composition of claim 7, further comprising one or more antibiotics embedded in the polymer.

12. The pharmaceutical composition of claim 11, wherein the one or more antibiotics comprise gentamicin.

13. The pharmaceutical composition of claim 9, further comprising one or more antibiotics embedded in the polydopamine.

14. The pharmaceutical composition of claim 13, wherein the one or more antibiotics comprise gentamicin.

15. A medical or industrial device or surface thereof, comprising the peptide nucleic acid or combination of peptide nucleic acids of claim 1.

16. The device or surface thereof of claim 15, the peptide nucleic acid or combination of peptide nucleic acids is comprised in one or more coating containing.

17. The device or surface thereof of claim 16, wherein the one or more coating further comprises polydopamine and/or one or more antibiotics.

18. The device or surface thereof of claim 15, chosen from a wound dressing or transdermal patch, medical grade tubing, dialysis equipment, IV solution bag, infusion pump, infusion port, catheter, central line, port, drain, endotracheal tube, tracheotomy tube, ureteral stent, biliary stent, ventriculostomy catheter, chest tube, gastric tube, intestinal tube, nephrostomy tube, prosthetic joint and other orthopedic device, vascular stent, stent graft, vascular graft, guide wire, balloon, suture, staple, filter, cerebral aneurysm filler coil, mesh, pacemaker or other cardiac device, prosthetic valve, anastomosis device, vertebral disk, bone pin, suture anchor, hemostatic barrier, clamp, screw, plate, clip, sling, vascular implant, tissue adhesives and sealant, tissue scaffold, myocardial plug, pacemaker lead, abdominal aortic aneurysm graft, embolic coil, dressing, bone substitute, intraluminal device, vascular support, or medical instruments such as a probe or needle.

19. The device or surface thereof of claim 15, chosen from an HVAC system, air or water filter, water purification system, industrial tubing or pipe, ship, industrial or municipal waste water distribution system, industrial potable water system, industrial or power plant cooling system, pulp and paper mill, industrial food and dairy processing facility, pharmaceutical or chemical manufacturing system, cosmetics manufacturing system, petrochemical pipeline, irrigation system, or aquatic equipment.

20. A method comprising:
applying, coating, or embedding to one or more surface, medical device or industrial device or system, one or more peptide nucleic acids designed to target or hybridize to one or more bacterial genes comprising rpoS, rsmA, amrZ, and motA.

21. The method of claim 20, wherein the one or more surfaces are chosen from countertops; tables; lavatory surfaces; equipment such as stethoscopes, ultrasound equipment, monitors, ventilators, extracorporeal life support machines; patient beds or bedding; wheelchairs; stretchers; gloves or other garments; sponges, wipes, pads, or mops; packaging materials of sterile medical or hospital supplies; or sutures or wound dressings.

22. The method of claim 20, wherein the medical device is a wound dressing or transdermal patch, medical grade tubing, dialysis equipment, IV solution bag, infusion pump, infusion port, catheter, central line, port, drain, endotracheal tube, tracheotomy tube, ureteral stent, biliary stent, ventriculostomy catheter, chest tube, gastric tube, intestinal tube, nephrostomy tube, prosthetic joint and other orthopedic device, vascular stent, stent graft, vascular graft, guide wire, balloon, suture, staple, filter, cerebral aneurysm filler coil, mesh, pacemaker or other cardiac device, prosthetic valve, anastomosis device, vertebral disk, bone pin, suture anchor, hemostatic barrier, clamp, screw, plate, clip, sling, vascular implant, tissue adhesives and sealant, tissue scaffold, myocardial plug, pacemaker lead, abdominal aortic aneurysm graft, embolic coil, dressing, bone substitute, intraluminal device, vascular support, or medical instruments such as a probe or needle.

23. The method of claim 20, wherein the industrial device or system is an HVAC system, air or water filter, water purification system, industrial tubing or pipe, ship, industrial or municipal waste water distribution system, industrial potable water system, industrial or power plant cooling system, pulp and paper mill, industrial food and dairy processing system, pharmaceutical or chemical manufacturing system, cosmetics manufacturing system, petrochemical pipeline, irrigation system, or aquatic equipment.

24. The method of claim 20, wherein the one or more peptide nucleic acid is chosen from a nucleobase sequence that has at least about 80%, 85%, 90%, 95%, 96%, 97%, 98% and 99% sequence identity to a nucleobase sequence chosen from SEQ ID NOS: 1-4 and 6-10, or any reverse, reverse complementary, or complementary sequence thereof.

25. A method comprising:
administering to a patient one or more peptide nucleic acids designed to target or hybridize to one or more bacterial genes comprising rpoS, rsmA, amrZ, and motA.

26. The method of claim 25, wherein the administration is by topical administration.

27. The method of claim 26, wherein the topical administration is to a burn wound, diabetic foot wound, mucosal surface, body cavity, or ear canal.

28. The method of claim 25, wherein the administration is by intrapulmonary administration.

29. The method of claim 25, wherein the one or more peptide nucleic acid is chosen from a nucleobase sequence that has at least about 80%, 85%, 90%, 95%, 96%, 97%, 98% and 99% sequence identity to a nucleobase sequence chosen from SEQ ID NOS: 1-4 and 6-10, or any reverse, reverse complementary, or complementary sequence thereof.

30. A method of preventing the formation of a biofilm on an abiotic or biotic surface, the method comprising hybridizing or causing to hybridize one or more peptide nucleic acids (PNAs) to one or more bacterial genes comprising rpoS, rsmA, amrZ, and motA of one or more bacterial organisms present on the abiotic or biotic surface.

31. The method of claim 30, wherein the causing to hybridize comprises applying, coating, embedding, or administering the one or more peptide nucleic acids to the abiotic or biotic surface.

32. The method of claim 30, wherein the one or more bacterial organisms are chosen from P. aeruginosa, P. mirabilis, P. vulgaris, E.coli, E. cloacae, and K. pneumoniae.

33. The method of claim 30, wherein the one or more peptide nucleic acid is chosen from a nucleobase sequence that has at least about 80%, 85%, 90%, 95%, 96%, 97%, 98% and 99% sequence identity to a nucleobase sequence chosen from SEQ ID NOS: 1-4 and 6-10, or any reverse, reverse complementary, or complementary sequence thereof.

34. The method of claim 20, further comprising applying or administering to the one or more surface, medical device or industrial device or system polydopamine and/or one or more antibiotics.

35. The method of claim 25, further comprising administering to the patient polydopamine and/or one or more antibiotics.

36. The method of claim 30, further comprising applying or administering polydopamine and/or one or more antibiotics to the abiotic or biotic surface.

37. A kit comprising a vessel and the peptide nucleic acid or combination of peptide nucleic acids of claim 1 disposed within the vessel.


SEQUENCE LISTING (36 sequences)

SEQ ID NO: 1 - DNA, 13 bp, synthetic construct
  misc_feature 1..13: note = synthetic peptide nucleic acid
  source 1..13: mol_type = other DNA
ggataacgacatg

SEQ ID NO: 2 - DNA, 13 bp, synthetic construct
  misc_feature 1..13: note = synthetic peptide nucleic acid
  source 1..13: mol_type = other DNA
ggagcagctgatg

SEQ ID NO: 3 - DNA, 12 bp, synthetic construct
  misc_feature 1..12: note = synthetic peptide nucleic acid
  source 1..12: mol_type = other DNA
gaaaggaatgct

SEQ ID NO: 4 - DNA, 12 bp, synthetic construct
  misc_feature 1..12: note = synthetic peptide nucleic acid
  source 1..12: mol_type = other DNA
gcaaagaatgct

SEQ ID NO: 5 - DNA, 12 bp, synthetic construct
  misc_feature 1..12: note = synthetic peptide nucleic acid
  source 1..12: mol_type = other DNA
gaatggtatgct

SEQ ID NO: 6 - DNA, 12 bp, synthetic construct
  misc_feature 1..12: note = synthetic peptide nucleic acid
  source 1..12: mol_type = other DNA
aatgtatgcgcc

SEQ ID NO: 7 - DNA, 12 bp, synthetic construct
  misc_feature 1..12: note = synthetic peptide nucleic acid
  source 1..12: mol_type = other DNA
gtcatatgagca

SEQ ID NO: 8 - DNA, 12 bp, synthetic construct
  misc_feature 1..12: note = synthetic peptide nucleic acid
  source 1..12: mol_type = other DNA
gcgccatgacga

SEQ ID NO: 9 - DNA, 14 bp, synthetic construct
  misc_feature 1..14: note = synthetic peptide nucleic acid
  source 1..14: mol_type = other DNA
cctcatgtcaaaaa

SEQ ID NO: 10 - DNA, 14 bp, synthetic construct
  misc_feature 1..14: note = synthetic peptide nucleic acid
  source 1..14: mol_type = other DNA
tcgcgtgttagtac

SEQ ID NO: 11 - protein, 10 aa, synthetic construct
  REGION 1..10: note = synthetic peptide
KFFKFFKFFK

SEQ ID NO: 12 - protein, 14 aa, synthetic construct
RXRRXRRXRRXRXX

SEQ ID NO: 13 - protein, 27 aa, synthetic construct
GWTLNSAGYLLGKINLKALAALAKKIL

SEQ ID NO: 14 - DNA, 13 bp, synthetic construct
  source 1..13: mol_type = other DNA
catgtcgttatcc

SEQ ID NO: 15 - DNA, 13 bp, synthetic construct
  source 1..13: mol_type = other DNA
catcagctgctcc

SEQ ID NO: 16 - DNA, 12 bp, synthetic construct
  source 1..12: mol_type = other DNA
agcattcctttc

SEQ ID NO: 17 - DNA, 12 bp, synthetic construct
  source 1..12: mol_type = other DNA
agcattctttgc

SEQ ID NO: 18 - DNA, 12 bp, synthetic construct
  source 1..12: mol_type = other DNA
ggcgcatacatt

SEQ ID NO: 19 - DNA, 12 bp, synthetic construct
  source 1..12: mol_type = other DNA
tgctcatatgac

SEQ ID NO: 20 - DNA, 12 bp, synthetic construct
  source 1..12: mol_type = other DNA
tcgtcatggcgc

SEQ ID NO: 21 - DNA, 14 bp, synthetic construct
  source 1..14: mol_type = other DNA
tttttgacatgagg

SEQ ID NO: 22 - DNA, 14 bp, synthetic construct
  source 1..14: mol_type = other DNA
gtactaacacgcga

SEQ ID NO: 23 - DNA, 1069 bp, Pseudomonas aeruginosa
  source 1..1069: mol_type = genomic DNA
cgggcgtgtccagcgggaaaggaatcgcccgggcttgagtcgaactcatgcaagggataacgacatggcactcaaaaaagaagggccggagtttgaccacgatgatgaagtgctcctcctggagcccggcatcatgctggacgagtcgtctgccgacgagcagccttctccccgggcaactccaaaagccaccacttccttctcttccaaacaacacaagcacatcgactacacgcgcgcgttggacgcaacgcagctgtatctcaacgaaatcggtttctcgcccctgttgacgcccgaagaggaagtccacttcgctcgtctggcgcagaagggcgatcccgctggtcggaagcggatgatcgagagcaacctgcggttggtggtgaagatcgcccggcgctatgtcaatcgcggactgtccctgctcgacctgatcgaggaaggcaacctaggcctgatccgcgccgtggagaagttcgatccggagcgcggattccggttctcgacctacgccacctggtggatccgccagaccatcgagcgggccatcatgaaccagacccggaccattcgcttgccgatccatgtggtcaaggagctcaacgtctacctgcgtgcggcgcgggaactgacccacaagctcgaccacgaaccttcacccgaagaaatcgccaacctgctggagaagccggtcgccgaggtcaagcgcatgctcggcctgaacgaacgggtgacttcggtagacgtctctcttggtccggactcggacaagaccctgctggatacgctcaccgacgatcgccccaccgatccgtgcgagctgctgcaggatgacgatctcagcgaaagcatcgaccagtggctgacggaactcaccgacaagcagcgtgaggtggtgattcgccgcttcggcttgcgcggtcacgaaagcagcacgctggaagaggtcggccaggaaatcggcctgacccgcgagcgggttcgtcagatccaggtcgaggcgctgaagcgcctgcgggagattctggagaagaatggcctgtcgagtgacgcgctgttccagtga

SEQ ID NO: 24 - DNA, 1201 bp, Proteus mirabilis
  source 1..1201: mol_type = genomic DNA
gtttacctaaattatcaagggtaggagcagctgatgagccaaagtacgctaagagtagacgagttatatactgatgatattgatgagaacatggaagagttcgatgaagcagccgcaacaaaagaggctgaaaatgaatctgacgctcaagatgaaattgaactcattgaaggagtcaatcagcgtgcgttagatgcaactcagctctatttaggagaaattggctattctccattgttgagtgctgaagaagaagtattctttgctcgtagagcgttacgaggtgatatcccatcaagacaacggatgatagagagtaatctacgtttagttgttaaaatatctcgccgttatacgaaccgagggttagctcttcttgacttaattgaagaaggtaatttaggccttattcgtgctgttgaaaagtttgatcctgaaaaagggtttcgtttttcaacctatgccacatggtggatacgtcaaactattgaacgtgccattatgaaccaaacccgcactattcgtctccccattcatatcgttaaagaactcaatgtctatttaagaaccgcaagagaattagcacataagctcgatcatgagccaagtgttgaagaaattgcgcaaacgctggataaaccggtcgatgatgtcagcaaaatgctacgtttaaatgaacgtatttcttctgttgatacaccgattggtggggattcagataaagccttactagatattatttcagacgaaaatgaagaagggccagaagcaaccattcaaaataataacctcaaggaaaatatcgtaaaatggttatttgaattaaatccaaaacagcgtgaagtattagcgcgtcgctttggattactgggttacgaggctgaaacattagaagatgttggtcgagaaattggtttgacaagagagcgggttcgtcagatccaagttgagggattgcgtcgtttaaaagatattctgcaagggcaaggattatgcatagaggcactttttaagatgaactagttgctttaggtgctcaatgcgttatcaagccgattagttaaattaactaatcggcttttcttttgttatttatttagctcttttaagtgatagagcaattccaacgcttgttttggtgttaatgaatccggatcaattttatttaatgctaattcaacttcagaaggctcaatatctg

SEQ ID NO: 25 - DNA, 1051 bp, Proteus vulgaris
  source 1..1051: mol_type = genomic DNA
caagggtaggagcagctgatgagccaaagtacgctaagagtagacgagttatatacagatgaaattgaagagaacatggaagagttcgatgaagttgctgcaaaagaggcagaaaatgagtctgatgctcaagacgaaattgaactaattgaaggagtcaatcagcgtgcgcttgatgcaactcagctttatttaggagaaattggttattctccgttgctgaccgctgaggaagaagttttttttgcccgtagagcgttacgtggtgacattccttcaagacaacgaatgatagaaagtaaccttcgtttagttgttaagatatctcgtcgttatacaaacagaggtcttgccctgcttgatttgattgaagaagggaatttaggactgatccgtgcagttgaaaaattcgatcctgaaaaaggttttcgtttttcaacttatgcgacatggtggatacgtcaaaccatagaacgtgccattatgaatcaaactcgtaccattcgtctgcccattcatatcgttaaagagctgaatgtttatttaagaactgcaagagaactagcacataagcttgatcatgagcctagtgttgaagaaattgcacaaacacttgataaaccagttgaagatgtaagtaagatgttgcgtttaaatgaacgtattacttctgttgataccccaattgggggcgattcagataaagccttactcgatattatctctgacgaaaatgaagaagggcctgaggcgacaattcaaaataataacctcaaggaaaatatcataaagtggttatttgaattaaatcctaagcaacgtgaagtattggcgcgccgttttggattattaggctacgaggctgaaactttagaagatgttggtagagaaattggtctgacaagagaaagagttcgtcaaatccaagttgaaggattacgccgactaaaagacattctgcaaagtcaggggttatgtattgaagctcttttcaaagcataactagcctttatttctgtctacagcttccaatatattgttattgccaaag

SEQ ID NO: 26 - DNA, 221 bp, Pseudomonas aeruginosa
  source 1..221: mol_type = genomic DNA
cgtgaggagaaaggaatgctgattctgactcgtcgggtcggagagaccctgatggtaggtgacgacgtcaccgtgacggtactgggtgtcaaagggaaccaggtgcgcatcggcgtcaacgcgccgaaggaagtcgccgtacaccgggaggaaatttaccagcgcatccagaaagagaaagatcaagagccaaaccattaatttttatctaattttccctt

SEQ ID NO: 27 - DNA, 210 bp, Enterobacter cloacae
  source 1..210: mol_type = genomic DNA
ttttaatctttcaaggagcaaagaatgctgattctgactcgtcgagttggtgagaccctcatgattggggatgaggtcaccgtgacagttttaggggtaaagggtaaccaggtacgtattggtgttaacgctcctaaagaagtatctgtccaccgtgaagagatctaccagcgtatccaggctgaaaaatcccagcagtccagttactga

SEQ ID NO: 28 - DNA, 231 bp, Klebsiella pneumoniae
  source 1..231: mol_type = genomic DNA
tttcaaggagcaaagaatgctgattctgactcgtcgagttggtgagaccctcatgattggagatgaggtcactgtgacagttttaggggtgaagggtaaccaggttcgtattggcgtaaacgcccctaaagaagtgtctgttcaccgtgaagagatttaccagcgtattcaggctgaaaaatcccagcagtccagttactaaggtttccgcgcctcgctgtgcagtgaggc

SEQ ID NO: 29 - DNA, 231 bp, Escherichia coli
  source 1..231: mol_type = genomic DNA
atctttcaaggagcaaagaatgctgattctgactcgtcgagttggtgagaccctcatgattggggatgaggtcaccgtgacagtattaggggtaaagggcaaccaggtacgtattggcgtaaatgccccgaaggaagtttctgttcaccgtgaagagatctaccagcgtatccaggctgaaaaatcccagcagtccagttactaatctttccgcgtctcatctttatcggt

SEQ ID NO: 30 - DNA, 211 bp, Proteus vulgaris
  source 1..211: mol_type = genomic DNA
tcaaggagcaaagaatgcttattctaactcgtcgagttggtgaaacgcttatgataggcgatgatgtaaccgtaaccgttttaggggttaaaggtaatcaggtacggattggcgtaaatgcacccaaagaagtatctgtccatcgtgaagagatctatcaacgaattcaggccgagaaaactcagcctaccgataactactaaacaaattc

SEQ ID NO: 31 - DNA, 339 bp, Pseudomonas aeruginosa
  source 1..339: mol_type = genomic DNA
caggttcaatgtatgcgcccactgaaacaggcaactcctacctactccagccgtaccgctgacaaattcgtcgttcgtctgcccgagggcatgcgtgagcagatcgcagaagtcgctcgcagccatcaccgcagcatgaactccgagatcatcgcccgactcgagcagagcctgctccaggaaggggcgctgcaagacaatctcggtgttcgcctggacagcccggaactcagcctgcacgagcgcgagctgctgcagcgtttccgccagctgacccaccgtcagcagaacgcgctggtcgccctgatcgcacacgatgcggagctggcccaggcctga

SEQ ID NO: 32 - DNA, 456 bp, Enterobacter cloacae
  source 1..456: mol_type = genomic DNA
ttggttctagaaccaatttaacaccatttattttgatgtcaagttggtgctattgtttagcgaacatctttacgttgaggtcatatgagcaagttccctagtcatgaaatggataggttcaatatcaggcttcctgcaggaatgcgggatgctatagctgaacgagccaaacgtaacggcagatcaatgaactcagagattgttcagatactggaagatgccttgaatgcagaaaatacactcggggaaatagcagataaaatcaacagcgtctcggttccgctaaatgttgatgcgctagttcaacttcaagcccaggttatcgccatgcaaaaagaaatacaggaaaagttcagagagcagaacgaaaagttgagagaactgctaaacaaaaaaaccacctgacggtgggttaatttttgccttttttggaccatattgactactcataaaatg

SEQ ID NO: 33 - DNA, 408 bp, Enterobacter cloacae
  source 1..408: mol_type = genomic DNA
tttgacaccattgatgttaatttaacaccattgcttatggtgtcaagttaatcctatcatgataaaaatttgtatcattagagatgcgccatgacgaagtacccgagccagttgcaggataaattcaacctccgcctccctgacgggatgcgcgatgccattgcagagcgagcaaaatctaacggcagatcgatgaactcagagatcattcagattcttcaggatgctatagatgacagttctagtctgtccaagcgtatggatcttgtagcgttcataaacggtgataaaggcaattcaaaaactgatgatatcgctttgaagctggcaggatatgtgcagaaacgcacagaagaatttcaacacaaactactagaagatcttaaagtaatccttaaaatcgaatga

SEQ ID NO: 34 - DNA, 858 bp, Pseudomonas aeruginosa
  source 1..858: mol_type = genomic DNA
gtcctcatgtcaaaaatcatcggcatcatcgtcgttttcgccagtgtcctcgggggcttcctgctctccggcggcaagatcggcgccatcatccagcccttcgaggtcctgatcatcggtggcgcggcactgggggcgttcctccagtccaacccgggcagcacgttcatggtggtgctgaagaaagcgccgaagatgttcagcaaccgcttcacccagacctactacctcgaggtgctgggcatgctctacgagatcctcaacaagagccggcgcgaagggatgatggcgatcgaggcggacatcgaagaccccgcggccagccccatcttcagcaagtaccccggtgtgctgaaggatgagcggatgaccgcctatgtctgcgactacctgcgcatcatgtcctccggcaacatggcgccgcacgagctggaaggcctgttcgacatggagctgtccagtctcaaggaggacctggagcatccgtcccacgcggtgaccaaggtcgccgacgccctgccgggcttcggtatcgtcgccgcggtactcggcatcgtcatcaccatggcgttgcttggcgaaggcagccaggcggagatcggccaccacgtcgccgccgccctggtgggcaccttcctcgggattctcgccgcctacggcttcgtcggcccgctggcgggcgccctggagcacgatgccaaggaagagttgaacctgttcgaggcgatcaaggcctgcctggtcgcttccgcttccggcatgccgccttcgctggccgtcgagttcggccgcaaggtgctgctgcccgcccatcggccgaccttcgccgagctcgagcaggccgtgcgcggccgctga

SEQ ID NO: 35 - DNA, 906 bp, Proteus mirabilis
  source 1..906: mol_type = genomic DNA
aagggatatcgcgtgttagtactcttaggatatatcgtggtgatttgtgctgtcctcggagggtttaccctcgtaggtggacatctaggcgcattatatcaacccgctgaatttttaattatttttggtgccggtatcggtgcttttgttgtaggtaataatggtcgtgcgatcgtatcaacggtaaaaatcctaccgaaattacttcgtaacactaactacaataaagcgatgtatatggatctaatggcattaatgtttcgtttgctgtctaaagcgcgccaaaacggcatgttggcattagagagagatattgaaaatccacaacaaagtgacattttttcccaatatcctcgcattatgaaagacgtttatatgctcagttttattactgattatatgcgtttgatggtgacggggaatatgaatccttatgaaattgaagcgctaatggacgaagaaattgccacttttgaagaagaaagtgaagtacctgcggcggggctctctacgatgggcgattcattacccgcatttggtattgtggccgcggttatgggggtggttaatgcgctaggtgctgccgatagacctgctggggaaatgggggcacttattggtcacgcaatggtaggtactttcttaggcattttattagcttatggctttgtttcaccattagcctctcgtatcagacaacgctctagtcaacaaatgaaaatgatggagtgcatcaagaccaccctgttatctagcatgaatggatatgcaccacagatagcggttgagtttggtcgtaagaccctctttctggcagacagaccttcatttattgaattggaagaacatgtacgtcaggtacggacaccgatgcaggcaaatcctgatgcaatgaaagaagagtaa

SEQ ID NO: 36 - DNA, 951 bp, Proteus vulgaris
  source 1..951: mol_type = genomic DNA
ctataacgaaattagctaaagggatatcgcgtgttagtactcttaggatatatcgtggttatgagcgccgtcatcgggggatatcttctcgtcggtggtagtttaggcgccttgtaccagccagccgaatttataatcatctttggtgccggtatcggtgcttttattgtgggtaataatggtagagcaattgtatcgacaatgaagatcctaccgaaattacttcgtagtacaaattacaataaagcgatgtacatggatttaatggcactcatgtttcgtttattgtcaaaagctcgtcaaaacgggatgttggcattagagagagatatcgaaaatccacaacaaagtgacattttttcccaatatcctcgcattatgaaagatgtatatatgctcagttttattactgactatatgcgcctgatggtgacagggaatatgaatccttatgaaattgaatctctgatggatgaagagattgcaacttttgaggaagaaagtgaagtgcctgcaacaggtttatcggcgatgggggattcattacctgcgttcggtattgttgctgcggttatgggggttgttaatgcgttaggtgctgcggatagacctgctggcgaaatgggcgtattaattggacacgcaatggttggtactttcttaggtattttattagcttatggctttatttcaccattagcttctagactcagacagcgctctagtcagcaaatgaaaatgatggagtgcatcaaaacaacattgttatctagcatgaatggatatgcgccacagatagctgttgaatttggacgtaaaaccctctttctggcagacagaccttcttttattgagttggaagaacatgtacgtcaggtacggacaccaatgcaggcaaatcctgatgcaatgaaagaagagtaattatgaagagtaattcacaaatcattc